SEQUENCE LISTING (16 sequences)

SEQ ID NO: 1 - DNA, 1657 bp, Oryza sativa
  CDS (69)..(572)
ggtcgtcgacccttcgtcgagccgccgccgccgccgccgatccttctcctcccacctcgcccgcgccgatggagcagctcaacgccaagctctacgacaagtacaccgccctcaagaagcggaagctgctcgacgaggggctggaccagaagcgggctgccgacatcaaggagctccagcaagccatgaaagattgggtcgccgacctccagggcgagaacgagcgcctcattgccaagctgacccaaaaggagcagcagcttgtggaggtccagacacttttgctcgatgagactaggaagacaaaggagctcaatagtgaaatattgaagcttcagtgtctcctggctgagaagaatgatgcaaaccatatagctacagggtctcctgacacaaccgctgaaatgattatagaaaatcaaactccaattccacctgcaaagaagacaccaaagtcaaagagcagggagagaaatatccacttcattgaaaaagcgactgttccttgtaatggctttcaggaggaaggaagagatcttgattcctgtagaaggcatatgagcatctcaggttagtggtaatatacacacatgcagtagtgaattgtttgaacttaaaagtatgttgtttgcagggagtgcgacagaagaaagctccagtacctgtatgtttcatatgctggctgaatcagtagttgggatgaaattttcagtgaagaaccaaacagaaggattttcactttcagtctcccatgaagctagtggtatttggaacatcaaattttatgctattattttagccacagcatagtactggaatatctcatcatagttattattcatcatgttttacttgtgttcatttctctttcacattcatttgattttgcatatagccacctccatgtttgccaagctagctgagtattcaagcaaacagttatgcaaagaacaatcttcaacctgaaaaatgtcctttaaaccaccagaagagttgtgtgaatacatattttaatgctttaatatctttgtcaagcatgtacacatctctttggagcttagccgtttgtagcagaacttgtataccagcatacagtgtagttacatgttgggtgcagaacctgaggatcatgtcaccaatgttagaacatttgatttgtaaagatcataatcaagtgtaaggaacgaattacatttctaactgaattttttaaactgttgtaggctacagtttcaccctgacatgggtagaccaacctggtggcggtgaatggtcatatcagtactcgtcgttgggcaccctggacaggattgctatgggctggatgaaggaggacataaaattcagcagggccatgtgtcctgtattctttcagcagatatcgcgcatcctcagacaaggctaagctcaactgggaagcttagtttggattttagaggtcctgacttttgctagacagtggactgaaactgacttgttttaggagtgacacgaggctttatgtatcctttcgtttggcctttgttgttcctgttgtgaacataacaattagcaagtgattcgagatgttggtcgcctggttgccgtatgtctttatgtatagcccctgatttgaactctgatggttctgaaaataagagcttcgacattgctgtcc

SEQ ID NO: 2 - protein, 168 aa, Oryza sativa
MEQLNAKLYDKYTALKKRKLLDEGLDQKRAADIKELQQAMKDWVADLQGENERLIAKLTQKEQQLVEVQTLLLDETRKTKELNSEILKLQCLLAEKNDANHIATGSPDTTAEMIIENQTPIPPAKKTPKSKSRERNIHFIEKATVPCNGFQEEGRDLDSCRRHMSISG

SEQ ID NO: 3 - DNA, 1048 bp, Oryza sativa
  CDS (76)..(960)
gatacagcctagaacaactgcttgccaaaaaaggaaaagaaaagaaaacacttagtgcagaaactgggaattgcaatggcgggagcaagatccgttggttctctcccagtgcctaatgtgcaggcgcttgcagaaatttgcaatgatccagatgaacacatacctgaaaggtacatcagaccagaggctagttctgaggaggtcatcaacaactaccaaggtgacatggcgattccgatcatcgatctcaagaaattgctttgtccgcaatcatcagaagaggagtgtgtgaagctgagatctgcctgccagtattgggggttctttctgctcattaaccatggagtgccagatgaagtgattgcaaacctgaagagagacattgttgatttctttagccagccactggacaccaagaaggagtacacacagctaccgaatagcctagaaggctatggacagagctttgttttctctgaggaccagaaacttgactgggcagacatgctgtatcttcatgtccatcctagcgattcaagggacctaaggttctggcccacttctccagcatctttcaggcaatccattgatgcatactcatcagagactaaaagcctggcactctgcttgtttgagttcatggctaaggctgtgggcgctaagccagagtcacttttagatttatttgaagagcagcctcgaggcttaaggatggcctattacccaccatgccggcaagctgacaaggtgatgggcctttcgccacactctgatgcgggtggcctgacactgctgctcgagattaacaatgtgcagggcctgcagatcaagaaagatggcaagtggttctccatagatgctccaaatggtgcacttattgctaacattggcgacacacttgaggtaatgaccaataacaaattatgctttcacatgtcgaaggaccttgatgtatttggatgagttcaataaaagctcctattatctagaaaacttgtcaaagcaccaaacaaaatacctaagttcaaattatttatattcttgagtc

SEQ ID NO: 4 - protein, 295 aa, Oryza sativa
MAGARSVGSLPVPNVQALAEICNDPDEHIPERYIRPEASSEEVINNYQGDMAIPIIDLKKLLCPQSSEEECVKLRSACQYWGFFLLINHGVPDEVIANLKRDIVDFFSQPLDTKKEYTQLPNSLEGYGQSFVFSEDQKLDWADMLYLHVHPSDSRDLRFWPTSPASFRQSIDAYSSETKSLALCLFEFMAKAVGAKPESLLDLFEEQPRGLRMAYYPPCRQADKVMGLSPHSDAGGLTLLLEINNVQGLQIKKDGKWFSIDAPNGALIANIGDTLEVMTNNKLCFHMSKDLDVFG

SEQ ID NO: 5 - DNA, 1335 bp, Oryza sativa
  CDS (219)..(989)
gagcgagccgagccgagagcgcaacaaaaggaaagcagggaaacctcgccgtcctctctctccatccatgccctagcacgcgccgcgcagcctacgagcgggagccacacctcgaggtcttcatctgttgcactcatgatttagcaccagctgaagtgacctctgagactgcgcggtgacgtagcctgttgctaggataaaaatctcagtagttaagcatggcggattctatggtgtcaagtttctgggggcctgttacttcaacaactgagctctgcgaggaaaattatgcacactcgtcatatattgcagaattctacaatactgtttctaatgttccatgtgttcttctggcacttgttggacttgtgaatgctcttcgccaaggttttgagaaacgattcagtgtcctgcacatatccaatatgatacttgctatcggcagtatgatcttccatgccaccttacagcacgttttacagcagagcgatgagactccaatggtgtgggagattctcctatatctttatgtactttattcaccagactggcattaccggagcactatgcctactttccttttcctatacggtgctgcttttgcagtagtccatttcctggtgcgattccaagtggtattcaagttgcattacgttggcctctgtctcctatgcatcccacggatgtacaagtactacatacaaactaaagacatggctgccaagcgtctagcaaagctgtgggttcttacgttgagcttggcgactctttgctggctatttgatcgaatgttctgtaagaagctttcacattggtacgtcaacccacaagggcacgcatggtggcatattctcatgggatttaactcatactttgcaaacacattcttaatgttttgccgagctcaacagcgtgggtgggagcccaaaattacccaccttttcgggttcttgccttatgtcaagattcagaaaccccaaaagagggaatgagttctctatttgtcagtgctgttctccaggaccttaaagccaacgccctcatgtgcttagacaagaagcagaggcattttttttgtatatgggcttccaagtgattacggactacagtgttatgatgcctcactagattttgttaacttttaactaacgcctgttttgtaccatacttaattgttgctcagagattagttggagtgttgtctcatagttttggtcattattttttgtttctctacatttgattttccgttctaataggactgaattttatttgttttttgtttgtcttgaatgttgtcgaacagttttagtcaagttactgcttcatatggcc

SEQ ID NO: 6 - protein, 257 aa, Oryza sativa
MADSMVSSFWGPVTSTTELCEENYAHSSYIAEFYNTVSNVPCVLLALVGLVNALRQGFEKRFSVLHISNMILAIGSMIFHATLQHVLQQSDETPMVWEILLYLYVLYSPDWHYRSTMPTFLFLYGAAFAVVHFLVRFQVVFKLHYVGLCLLCIPRMYKYYIQTKDMAAKRLAKLWVLTLSLATLCWLFDRMFCKKLSHWYVNPQGHAWWHILMGFNSYFANTFLMFCRAQQRGWEPKITHLFGFLPYVKIQKPQKRE

SEQ ID NO: 7 - DNA, 1631 bp, Oryza sativa
  CDS (1045)..(1266)
gttggtctaggctgtaactacctttttttatttactttatttgtttcctgctctatgtagtgacatgctgtgttatattgctgaattataataccatcaaatagtttggagaaagtactttattttttctgctggcatgtttgaacattcacaatactatatcgtgcatatgtacctctctgttttggtcttgtcagttaaaaacgaacggagatttcttgtgctctcttttttttagtaatttctggagcttaggtttctttagcgtttgaaaggaatgaggaattgataatgatcgagatgtcgtgttatcaacttttctttcttaaacaatatactagtcatactccttttcctgtatttacagtgtcaaatctctgtcagaggatgaatggatacttgacccggttatcattgctgatatatatgagcaacaggtatatatcttcacttccatcaatacactagaaactatgcacattatgatcttattaaaagagcttttgtgtatcaatggcgttacactttccactgtattgatcttttaaagagtgtcatttgttgttttcttataaagaattataggtgatattgttattagtacatgctgacccgttattgtagatttcatagttatcatctactaggctttttatacaagttgtctctatgatctgctctgcagctctggcttgtatggtagtaactgaagcaaaaagtcctgacttacaaaacaattgtgggtgtgactctgttagttgcagttgctggctattttcacatgaagctattttttactaaatatgattgatgtaattatatgtaaaaaggtaaaaattgtaacgaactgcttggaatgtctagtaaaactatcatagcgtccattctgcgttactttgatgtgcagttgtgtacccattcctgggtcatgctgaaacacaaattggcgtgatacgcttcattaattggtgattccattttcagggccttgatttcattgtcgatggaggtactagaattgctgacccgtctactgtggttgatatgacgggaagctaccctaccatcattcgtcagggaaaggtatgcattttcattaatggactaaagttatcctaccatatgactatactatcaagcaaaaggaaatttctcattctatcccctgacatttggcaaatgatatgtactgcatttaaacacaccgttatgtattatctcgtaatgcttatctatttcatatttgtgacctatttcgttgacttatgattggcataattagctaatactggatgcttcaactgcattttatgcggtttgtataaatgcacagaggcccttatgtcttccttttcctgctttagggccctaagctagattggatggtagcggaagatgaggaacaagagtcgcgaccaacatttgcgtataaagcagcttgagtcactcttcggctgtcctatcataggaggttgtaaacttgtaattacttcaccagagcctggtcttttgactcctgaatctgctactctcagctagaccagtatatatactacatgttgtaaatatgccagggaatgtaaccaggaatcgacatagttcaccagattattttcagttcactgagcctc

SEQ ID NO: 8 - protein, 74 aa, Oryza sativa
MTGSYPTIIRQGKVCIFINGLKLSYHMTILSSKRKFLILSPDIWQMICTAFKHTVMYYLVMLIYFIFVTYFVDL

SEQ ID NO: 9 - protein, 257 aa, Zea mays
MADSMVASFWGPVTSTTELCEENYAHSSYIAEFYNTISNVPCVLLALIGLVNAFCQGFEKRFSVLHISNMILAIGSMIFHATLQLLLQQSDETPMVWEILLYMYVLYSPDWNYRSTMPTFLFLYGAAFAVVHFFARFQVVFKLHYVGLCLLCIPRMYKYYIQTKDVGAKRLAKLWVLTLTLGTLCWLVDRVFCKKLSHWYVNPQGHAWWHVLMGLNSYYANTFLMFCRAQQRGWEPRITHLLGFLPYVKVQKPEKRE

SEQ ID NO: 10 - protein, 255 aa, Arabidopsis thaliana
MADGISSFWGPVTSTIECCEMNYAYSSYIAEFYNTISNVPGILLALIGLVNALRQRFEKRFSILHISNMILAIGSMLYHATLQHVQQQSDETPMVWEILLYMYILYSPDWHYRSTMPTFLFLYGAAFAIVHAYLRFGIGFKVHYVILCLLCIPRMYKYYIHTEDTAAKRIAKWYVATILVGSICWFCDRVFCKTISQWPVNPQGHALWHVFMSFNSYCANTFLMFCRAQQRGWNPKVKYFLGVLPYVKIEKPKTQ

SEQ ID NO: 11 - protein, 255 aa, Medicago truncatula
MAETISSFWGPVTSTIECCEKNYAYSSYIAEFYNTISNIPTILLAFIGLINAFRQRFEKRFSVLHVSNMTLAFGSMLYHATLQHVQQQSDETPMVWEILLYMYILYSPDWHYRSTMPIFLFFYGAAFAVVHSFVRFDIGFKVHYVILCLLCIPRMYKYYIYTADVCAKWIAKLYVATLLLGSLFWLCDRVFCKEISQWPVNPQGHALWHVFMGLNSYFANTFLMFCRAQQRGWSPKFVRLFGVLPYVKIEKPKSQ

SEQ ID NO: 12 - DNA, 30 bp, Artificial Sequence
  Description of Artificial Sequence Synthetic primer
gtacgtatttttacaacaattaccaacaac

SEQ ID NO: 13 - DNA, 30 bp, Artificial Sequence
  Description of Artificial Sequence Synthetic primer
ggattcaatcttaagaaactttattgccaa

SEQ ID NO: 14 - DNA, 1981 bp, Zea mays
gccgctgccgccggcttacggaaggtgtgatctccatcttcgggacgttcgccaccggagcacccactccttttcttccgttcacgctgtgcgaaacggatcatccaaaaaccagctatttgtatctggatctgatccaatttcaatttgtggtatacatgtattgtgtctacaaactatttttcgtttgtttttttgcaaaaatatttttcgtttgtctacaaacaatttcaattgtagtacacccatgtcctttacccgtgcacacccttgcgctgcctgctgcggttatcccatatttgttgtggagcgtgtgccgggggaatttggtttggatgtggttgggatcgaggtatactctagattagggttcgaagttctcatatgacctgtgtgatgtggtagattcgatgttagagtgtcgatgaaagttgtctctgagtttagttttgcacatgagtattgaatatttgcaggtgtttctctgagtttacttctacacatgtcaattattgagtattggtaggtatgattgtctatctcttgctaaggaaattgtttaggtgttctatgatagtgtttgactttagattgcctattttgaagggacactttcagcgacagtatttaatttggtgtgttgccagaagacctggctctgaattatctttctatttctttgattatcacagggaactctactaatagtatgctggattagcacgattaaactgaagtatgacaaatttgcatatcatccttgcaggttatttctttggcgcactcatgatttagcaccagccaaattgacctttaactctggtgacataagctgttgctagggtagcaaatcaagagctaagaatggcagattcaatggtagcaagcttctgggggcctgtgacatcaacgactgagttgtgcgaggagaattatgcacactcgtcatatatcgcagaattctacaataccatctctaatgtcccatgcgttcttttggcacttattggattagtgaatgctttctgccaaggttttgagaaacgatttagtgtcctgcacatatccaatatgatacttgctattgggagtatgattttccatgccaccttgcaacttctcctacaacagagtgatgagactcctatggtttgggagatccttctctacatgtatgtcctctattcaccggactggaactacaggagcacaatgccaactttcctgttcctgtatggtgctgcctttgccgtagtacatttctttgcccggttccaagtcgtattcaagctgcattacgttggcctctgcttgctctgcatcccccggatgtacaagtactacatacagacgaaagacgtgggtgcgaagcggctcgcaaaactgtgggtccttacactgacccttgggaccctctgctggctggttgatcgcgtcttctgcaagaagctttcgcattggtacgtcaacccgcaggggcacgcgtggtggcacgtgcttatgggcctcaactcgtactatgcaaacacgttcctgatgttctgccgggctcagcagcgcgggtgggagccgcggatcacgcacctccttggattcttgccgtatgtcaaggtccagaaaccagagaagagggaatgatttctgtctctgcttgaaaactgatgatattcagtcggccaccagtaagctaatgcccccaatgtgtgctcagacaagtccgtggcactcttgtatatgggcgcccaaatgattacggacttcaaatgttctgatgtcgcattagattttgtatcatacttaaatcatcgaacagagattgtcctagttttggccacctttcttttggtttcttcgcaattaccgtttggtggtaaattaggactgagttatttatttatttgtttgtcgttgaatggagtccagttgtactagttaccactgcacaagaagatacattcatccacatgatgacatgttaatt

SEQ ID NO: 15 - DNA, 1347 bp, Arabidopsis thaliana
gaaaaggaccaagagagagaagaagacgaaacgatcgtcagagatagagagagagaaaggtcatcacccattctcactcgcagaatatttgttctcatctgattcctccgatctgccatgtctcggcttttctaacgctaagaagtttcacagacgcggcgaaggagagggaggaaggagatagatctctttttccgtcgcgataataataaaaaataaacgtgtctagaggctgttactgaagagttgattcatacggagagaggatagaaatggcagatgggatatcgagcttttggggtcctgtgacttctactatagagtgttgtgagatgaactacgcctactcatcctacattgctgagttctacaacactatatccaatgtccctggaatcctattggctctcattggtcttgtcaatgcattaaggcaacggtttgagaagaggtttagcattcttcacatttcgaatatgatacttgctatcggcagcatgctctaccatgccactttgcaacacgtgcaacaacagagtgatgagaccccaatggtgtgggagatacttctatacatgtacatcctttactcaccagattggcattaccgaagtacaatgcccacttttctcttcctctacggtgctgcctttgccatagtccatgcttacctcaggtttggaattggtttcaaggtccactacgtgatactttgccttctatgcatccctcggatgtacaagtactacattcacacagaggacacagcagccaaaaggattgcgaaatggtatgttgctacgatcttagtgggaagcatatgctggttctgtgaccgtgtcttctgcaagacgatatctcagtggcctgtgaatcctcagggacatgctctgtggcatgtgttcatgagtttcaactcttactgcgcaaacacattcttgatgttctgcagagctcagcaacgtggatggaacccaaaggtaaaatactttctgggagttctcccttatgtcaagatcgagaagccaaaaacacaatgagaagaaaattggggataaaaaagcccagatgaagaagaagaagatcataaatatagtttttgttcatcgttttgtcagaatctcgacatggtttttttcttaattttattttttgaaagtgaaactctctagatacagtctagatttttttttttttttctcatctctgtattagctattgggaatgctcttaagaagaattgatttttaacttgctaaaaaagctcttgtaccttttggttttcctgtgtttggtgtgtgacatcatttcttcaactataactcgtgtaccataatacttaatgcc

SEQ ID NO: 16 - DNA, 96388 bp, Medicago truncatula
aagctttgatttcaccttgatttggatttagagctccgtttatcgaagattatgttgtctcgaattgttgtcttttttgctgataactccacaaaacaaagttggttattcccaagtgaaaaggagatttggaatacgctagaaatgcagaatttcgaacacaacatgttggctaaagccattttttcaaaatggagagaaccccaactcattctcccaaccaatcaatatatatgccttgggttcttaagatagcatgagcacgagcacaaggattacaaactaagagaaagggatgaaccttattttgtgttagttgtcttgtcactcatggatatagggggaagaattagacggaaattcagaaaagtataacaatgttaacctttgaaattataaaggaccgactttaaaaacaaataaaggacagatttaaagctttaaatttagggatctcgggataaattttgcctaaaaccaattacagtatgttgaaaattattttttactactatcactgttaacttagtttcttttgaaaatatgttaaaaccataaccttttgagaaaagggatttgtgttagcttgtgtaatttcggccacttaattggcttattgcaatagtttttgaaatagtttcatgttatgtcatacttttgaaaagattgagttattagaaaagggcaggtatgaacaaggaaaattgcttatctcccacacaaaattgctcactcccatacaaactaacgaaaatatctttaacgtaacgtgacttaagtcacagtcgatatcatcaacgtgagttaaatcacgttggacaattcacgctgcgtgagttaacaaaccatactcaaccaatatagggacttgattctctccatatgcaataatatgatactgcaactgtagtcgatccaattttgccaatgtagtcaatttcgaaccctatgttccatgttattattacaaattataacgattttagttttcagatattgtaattgtgtacacaaactttttttagtaccaaggaggactatttcttcccctacataaacgtttcctttgatgatttataactaatttataacaggatcgatagtttttttcccctttaaaaccttgatataaaatagtttttaataaaccaaaatttcaactacactttcatagagatcacacgacatatagcaaaatagatattgaaccacaactgcactttttcataaaacttgtttttaaaaaattagggctcaaccctaatagcgttaatgctctctagataaaagagggaagcataaaacaatcttgaagagtaacgaatgaaatataatatttatatgtgtttgcaagcgagaaacatatctcactatccaagccaattttgtgatattaaattagactcaacctatattctaaaataatatcaatgtctaccatcatttcaccaaccatattatctcacattaaaaataaaaaattaagatattaaaaaatgtttataaatagtgacactatttaacttatacaagcgaaatttgtgatttttttagtcaaactagtctaacaaaattgacacccggaataatcaaacatgataccttaatatgaacacattcaaaggtccgaagtgaacaccatcaaaccaaccaaagtgggttcacaaatttcagaaaaagtgcttttgatcaaacacaaagtaaactaacagaatcaagcaccaagaaaagctgctattcagtttatatgaaccacaactgcactttttcagttgcaaaacatcaaattcccaactagcctggaaactttcattttgaaatttcccagtgaaagcaattttgaagttccataagttaataataacatactcaaaactcacctagaaagggttacacaacatgttacaagtaaaaatgaggtactggtatgaaacttagaatcaaacaaccagtactgcaaaacctttggttgtcaaaagtaatgttaacagtctaaggccaagaaagcatccatatggcataaatatggtacatgtcctaaaactgcaacaaagtatataaaaaatcatcgccacttacaccaatctaaatcagaaaaacaaaactgagaaccaagaacgaaacactgataactcttccaaaatgtatcattaaagaaaaccttcatcatcttctactatcttcccttattggctttttggtttctcaatcttcacatatggcaaaacaccgaataaacgaacgaatttcggagaccagccacgttgttgagcacggcaaaacatcaggaatgtgtttgcaaagtaggaattgagtcccatgaaaacatgccataaagcatgaccctgtggattaaccggccattgggatatctccttgcagaaaactcgatcacataaccaaaacaaacttcctaatagaagagtggctacgtatagctttgctatccacttggcgcacacatctgcggtatagatgtaatacttgtacattcttggaatgcagaggagacagagaatgacataatggactttgaagccaatgtcgaaacggacaaatgaatggacaacggcaaaagcagcaccatagaaaaataggaagatgggcattgtactacggtagtgccaatctggagagtagagaatgtacatgtacagcagaatctcccacaccataggtgtttcatcgctctgctgttgcctaaattcaagcgcaacaaatgaagaataaatagttacccaaaaacaatagatattgattgaaaaacttcacgttcttataagctttgacaaacttaatagcctacgcattacgatagtgaacaaacgcctaatttcctaagagcttcagcagaagaaaaatgtaactaaaaaggattatatgcaagtgagcagtcatgcattttgaagcactgtaatatgagtgcaatatgaggagtgctaggaacactctaatactcactcacattgggtgaaatctatgtgggtaccactctttggaaattggttccacataaaatggtgagacccacttgggtttcacccaataagagagtgagtgttagaaagagtgtccctagcctttctcatacaatattatgcatatacaaagaaaattcccaatgattgtaaaaagttagctcttatagtaccaaaattgttcaataatcaaccctcaagcataatcaaacagctattgtttagtcaaaatattttattgatcaatctttctaacccaatcgaaataaaacccttcctcttagtagcaaaaataagtttgtctaacatatggaaataaagaaatacaaggggagaaaagggatgtagtcacaaaaaaattagccccctaactcaaatgcacaaatgtctcgtgtgcatcagatacagtcatgttcattagtaagacagctatgccggtggaaaacaactaaactaacatatctataaattgctattgatgtatggctgcatgaccataccaaaacccaattaatatttctggtttatttgaaaaatgctaacaagttcccttagggcattgtttaaagaatcaaaagaggtaacttttgtatagaaaatgatgtatttattacttccacaagtgtttgacttgtatttccaaacaaaattttcttttacgggttccttaaacaatgcccttagggaacttgtttgcatgacccttaatattttcgttacaaaacaaggctaatgcatgatgtatgttgtacaaacattttatacgatgcaaagcaactcaaaaacaaaatgaaaggaaacaactaaaagtttagatagtgcaaaagaagagagagatcaatacttacacatgttgtaaggtagcatggtataacatgcttccaaaggcaagcgtcatatttgatacatgaagaacactaaatcttttctcaaaccgttgtctaaaagcatttataagaccaatgaaagccaagagaatggtcggaatgttggatattgtgttgtaaaattctgcaatataagaagagtatgcataatttttttcacaacactcaatcgttgatgtaactggaccccagaagctagatattgtttctgccattctcctctacagccagatgatttgcttatctagatatttgtatttaaagctgcagatatcaataattcctgcaaatcacgtggcaacatgccaaaattcaatcagtcctgcaacttgtttatagggatggaaacaataaggcaggaagttttcgtagatcaagcacatcagcatgttaatcagtattattattaatatcgtatcgcggaaaatatcagatcgccaaaaattcgatatgatacatagcgtaagccacaaaacggttcaaataaataattatatataaaataaagcatgctacaaacaaataaaagctaaccaaacagaaattaaaaatgaattgatatacaacttaaaatctcaatagttcaaacaactagtagaacctggtgcatagttgcacacattattgcagaaagccataagcagaaattaaaagtgacttgaaatccaaaatctaaatagttcaaatcaactaataattggtagttctaatagtgataaaagatctttttttttatgaacaataaaagatctaattattctcctcttccccaacataataattgtcctcattttcagatagtgaggcatttccgtcaacttcttcaattgaattgtctgaatcaatttccttaaactctgcttctgattctaccaagacagggtcatcataggtttttggtattatatcatgggaaatactggattttacgaatgttgctcacacgaaacatatgagcgtgttttttttatgaccagctacatccactgcccctccttgttttctataaaataacaagttaaatactctttgactttttattaaaagagccactacacattcttcgctttcgtttgggaattttttcataagaagtagcaattcttttatattattatattgggcctacctatttgaccgacccatcggtttccaataaaatattataatattgggctttataaaaaaaatacattaaaagatacaagctaaaaattgtggaatttcaatatgcatgccatgactgatataccatatttgtgcaatctcgacgatattttggaaataccgaagttgctaaatcgtatcggattttgtatcgtctaccctaacttccgatacggaagagcagtatagcgctgatatgccggatatttaacaacactgatgttaattcacaaggaaatcatggcagctatgctaaaacaacacaacatgaaaaaacaacagaaaaattgacaattcattctagtcccaagcacggttttagatagtggtaagggttgtggtggcattgcagttcttgatattgcggaaaaccacagtcagatgcagaggacgcaatcacaatcatggtcagtttaagcaatatcaaaaaccgacatctcacatcctagatcgcagttgcggacctctatttaaaatacgggttcaaagggcaagataagtaagctattgaaagagaacctataaggatttacacttgattcattaccttaaaagaataacaatgtccatacatacgcaagacacaaacttaaaaaatcctaacaattgaagctgtaaattacctacttatgaaattcaaaatgaccaacaatgttcaaactgtgaattctgcaatttctaatacaccttattacacatacatgaaaataaaaataaaaggtaaacttatatcaaccacaataagtcactgccaaataacaaccacaaaacaaaaaacattgtaatttggtttcaaaaacataatatttgactaaattcatcataagcgaagatagatataatcagatcccacatggattaaaatctaaaaggtaaaaaaaaaaaaaaaaaaaaaaaaatcctcaaaataatgcagtccaattctgccaaaatttgcagagaagatccaatctgaatcgttttccgttcaaattaaacaaaagggtcagtgaaaaaacaaaaatttatagcattgtcaacaattttcttatgagttcgtgccaaataagaaaaaaaaacgcttcatgcttaggaaaaaaaacacacaaaaaacaacgacccagatggtgaaaatatataaggcaataataaaagcaatgagattgtggaaaagatgcatacttgaggagattgaagaagaaacaagtagatcgcatcctgggttgggttcacttctgcgtttgtttttgtttgtgtgttgaaaaatttaatattcggtcagattaacagaacaaaagatgggtcggatgatctcttttcctcgtacggcgtgagtgatcttaaacgacattaattttgggttaattaggtttttttgggtacaaaggttaattagatttttttatctaaatttaacaaacttttgattcttgtaatttttttttttagtatgaagccctttaaaactcgataagttttttctttttttaatatagtaaaatttataaatatggtttacaatagaacatttcaacacaatttaatctatgtcgacttcgtttggtgaaataaagtttgattgttgttgttcttatagattttacaaaagggcaatataaaaatatatttattttttgcggggagcaaaagggcaatgtacatattcccatgagcagaacaatatgcaggggtcgagatttgaacatatgattctcatcaacaacaattgaaattgatgaaattcacgtgggaacaaattaagtatgaactatacgctcaatcattgacgatatcatgcaaaccctaacaaagtggtgtcaacgcatatgaatacacaaacgatgaaattcatgtggaaacaaattaggtatgaactatacactaaaacaatgatgatatcgcgcaaaccccaacaaagtggtgtgtcaacgcatatgaatacacttaataaatatttgtctatatatacgcacacgatttttcctacctgcaagtgacattcggcatttgactcagacatattaatttcaaacatatgtgtcgcattctctttactttatttttagttaattaaaaatattaatttttaaaacattaattgtttccggtatatcatacttttagatgttaacttcaaatatagcgtattcttttcctctaaaaaaaggaagtgagatctatttcctttcttacggacggcaagttttcatactgtgtcattctcgtcgagcattggatcatatcactaaccaaggtttccaactaaaggctcaggcaacctatgagcttttgacgaggtccttgatgaatagttattcctatagaacttccaagtttcagtgacggatccagaaatttaaataagtggggtcaaataatataagagcgatgctatatagtgtgaaagaaaacatagcgaaagcatcccgaatcaaaactaaccaatcacaatttgttgtttggcaaaatacaaggaggtgtttacggaaaaaacagaagtcaagaaataaacctccaattaaaattagcattgtcgatgtgtatttgcttgacattctctcactagaaatagcatttctcatattataaatacttataaaaaataaattaagacataagtgataaaataatgacagtgtatattgaaaaagttagagattacgataattatactattgtatacttataaattaaagacaacttaatgcttttatgtaattgctcaacacactgtcttttaagttttaaaagtgttgtaaatgatattatgtgtgtatgtgagagatcaaacaatcattcagaataataatatccctagtattattgtgcatttgattctaatacaagcacaaaatttaaaactttataaaaattgagaccgcaaatgtcaaataacaataacacaatacaagacgaaaataggaatgtagggagaaacttatatttttgaaaaaagagaaaagaagagagatttagaaacagggagacacatagaaggaggtatacacttccatactaacatactaatgaagtttttcatattgtcgtagatgaaggaatattcatttgggcgaatggaaattgtcatattatggggactttagtctacttcatgtggggtcaacctatcaaaactactcattatttttcacgaaaatattgaaataactcattactagttcatttaaaataaatcaagtggggtcagctgcccccatttctcactatgtgcatccgtccctgccaagtttgccaaatcggctaatgtttgcatagtcatatcaagagtaaacaatgctaaattataggcgaagaggcagagacgagtgacaaagactctgatgactttgacaaaagatatattttattagacaaatgaaatattagttgtcttttgttttcttgaagatctgttagtgatttccttttgtatttagattgaaatctattcacatattaatcaaaggggcgaccatttgatcatcatgcatgtgggaccaagttttcataggataagtgggggtataaaagttctaagtaactatataaacttttctatctacattggctcacctgtgttacatgtttcatgataaaagaacataccaactagcgaatgcaaaatgagcataagctgaaaacaaaacattgtgcatcaaaacaaaaagttccattatgttcaaattaaaatggataaccacaaggtcagctatctagttaattttacgagaaaaagaaataaaattgtatgtggatccttctttgaagatctacatgaagagaaaacctcataatgataatacatcttcactagggtttggatgaggcaaaggagcagtgagacagataccgaggattagcctgttttccctgaaagacttattgttggtcgagaaagtcaaagtgtgaaagaaagaatgaaatggatgatgatttaactcgacatgaataggaatgtaaagtctcgcctcttatccacattgaagtgttatgattgagaaaatgatgtccaatagaaagaatttttaagtaacttgaggcttgatgtatttactacgaagtctaaaatattaaaatttgaaggaggagcgaggctcatgtttgatcagaggaagtaaggatgtcaaatgttgatttattaataaagtgcttgaatatcaagcatgaagttttctccgaggtagataaagaaaaatgaaagatttaaccgggaaaagaaatcgtctatattgaaataaaaacaacgatgacaattgcatgttgtggcaaggttacttgacagttcgtttataccttacaatgtaataatgacaatgttcacgctcaactttgatatttgcattgcatcttctcgtctctcaaagggaatctgataatacaatgtatgaggcatttgtacacacctaagaataattatcatgttaaccaacctcgacatataagtgattggattttaggtgatgaaacccctagccgatggaaggtaaggcggtcttggagcaccccgagcgagggaaattgattatttcacaagcctgctaatcaagttcattcgttataatggcctcatactaagtcaagaaaaagtgggacttgataataacacacaaaatgtcgtgaatgaggatatttagagtggaagatagggcgctacaacctcctaaacggagaaaactgatgcactacccgcaaaattagtcgagcagaaatttaacactgtaagtcacgtcatttggctaataagcaatagttttactataaatactcaatacatacattgttattaaaaaatccaatatcaaccaaacacactgatttcactttttttttttttttatcttttcaccttcttatctttaattttactttacacatttcagagtgtagctcaaaaaccccaaataattgtacttatcgctctactgtagaagttagagaatacacaactgatgaaaaaagtttcttaattaatcccaatcactttgaagtaaaggaatcatagttgaggacattcaatcgaacatcaactaaattcttgttacaccaacacatagtaaaacatcaaattatctcttttagttttagattttcgatagtaaaaattcacttatagttttaagtgttttaaattgatatggttccctggagtattagaatgcttggatcaaccgacttcaattcactattttagagagatactttgtagatacccaagccactttttgacaccttgagatataaagaaatagatagaatgatgtaataaatgtgatatattgatgatgtgataaaaagatagagatgaagagcaagtgagatgtttgaaaaataaaggatatttatatatcattttttataaatcttttactttcaagtgctgtcatgttttctttccgaaaaaaaaaagtgctctcatgtttacagcagcgaaaataatactatgatcatgattggttggttaggttaatccgcacatttattaataagtctttgctcttttctggactatttgtcgatgtttcatggttttttctttttcaaaatatgaaataaatgaaatttacttgtatagtatttttttttttaccaaattatcctctaactgaaactgattttgttgaactaaaatgagttttaaacaatggctctaaagttttgacgataccaatgtataaaagaacaattgaatgtgttaatgtgtgttcaagttttcaaaactacagagttaaattccgactctcgaatgcaaattttagttttgaaacatcaagactctgaacgcataagaaataatctgaagcaaaagagaaacttatatgactttgatcaaaatctgaataaaggtcaattagaaagtcaacacttagatcataagatatttttctctaataaacatctgaaggaatgaaaactctgaaggtgatgtcagcagctgaagactttgacccaacttgtcgtcttctgaaggaatgtcaatattagaatcagagtagtacaatttgtcttcttttgttcacgtgtatatacaaacaaagaaatttaaggacaaagagaatgtaacatatcattatattataagcaaaggaattcgaatgagtactcaacgacgttatccatttgaagaagcaaaaaaaggacttctatcagtttacaagtgtcattactctgtcaaattacatagcacacaaacacttagaaaaattctcacaaattttatatcttagaatttctgcatcaaatatcatttgaacttaatcttcttacacctctgattgtatatcaagtgtgaaacacaatatcttttatttaattaaacttatattatagaagtttatttcttgtgtaattgagcaaaggaagtctcttactatgtgtttgagcaagttgtaattttgtgtaattatagtgaagaacctttggaagtgcaaggaaaatggactactcttggcttgtgagagaaaccaatataatctgcccgtgtcatctctttctctcttctctctatgtttgtaattttattccgctccataactctctttcagactctgataaaaactctccttctgatcagtctctgaactatttttgataaacacttaaaaaagattcttaaaaaatcaacacaattcaaatttaaaatagtgataaaaggttttcttcatcgaaatgaaattatcctatatgtctttatatggtgcgtaaattagcatattttatacatatgagattttaaaaaaaattaaaagaatattaacacaataaatgtcctataacatttagtcacatttaattaaaaatatttcataaattccctcttttagaaaaagaaaaaaaatataaaaatcaacccctaccctccaaaatcttgcatccaagcataccttagttactcttgaaacaacaagtaatagtttcttttttaaaactttttaaagttgaaacaattattttttagaatttgagttcaaattctcacttaaacaatttttaaagagacattacttatacctcgcccaaactcaaattaatagaatcattccctttaaaaaccaaaggattaagatcgaagaaaacatttttgactcggttctcaataatgagactatacatgtttttgtcaatgacaaaagtgacaagtactacataaaactcacaaacaattgtaatattctgaaaaggaatagttgcgtagattttaataaaaaaaaatatgattaatttattaaaatatcattattaattatagttagtgacgtaattaaataattaaaatgtaataaataaggatatgttggtgaaaaaaaataataatttttttattgatattataaaatagataaataatttgatacaaaagaaaaaaagacatctattttgagacggatagagtactgaaatgtctattagcaaaaaaagaaaaagaaaagaaaataatgattcaagtgtcttttgtaagaagtgacattattggtcatttccaaaacaagaaagatgccgattttgaaaataagaaaaaaaataatttcatggtttgtcatcaacatttatctgcagaaaaagacaaaaatagatgtcgcaagctctaccagaaatggcatgggagattggagaagataatgattagaattcattgaatgaagacatttaattgaggttcattgaacctggtctggttgaacataaatcacctgcccaaacaaacacaacacctcccaatttccacatcccattataattataacttgaaatataacataacacatacaaattttatggtacatccattatatttaagtgtaccgatacattcactctttaaatttatagttaaatgagttattttttgaagaaaaatattattttttatcatatattataataactaaatcttattcatcaaaagtgttaacaggaaataattttttatttttttaaatttttatcactcataattgagaacattcattattttttacgataatatgtaaaaaaaaattcctatgattcttataaacaattaaatgatgttttttcttatgaaatgttgttctataaaatataaatattgacaaatagttatttattacataaaaaatgatcgttcatttataaaattaagaagcagaagtaccggtacaccttattttgtgagtgtaccgtagaagttcccaatataacatggtttttctttttaatcaaaggacttactttggttaatttgaataaatgtttttattttttatttatgtaaaatgaaattgtatttttttcattttagagttagattttatagggtttttgttacattcttgaaattaagtctatgtttttatgttactcttaaaattaaaatatacttaactctcttatcattctcttaatccaaaaaaaaattcatttatcattctctctaaacttaactctcttaaaagagtataaacttttttctatatcttcccttcaacctcccagacatacctacatgtctaaattattctgcgaataaaataagacaagaggatccagatagtgagatggatcgtgctcagcccctctgtgtagattttaaaagtgtactcatcaccctatgtcaagtgggaaaaacttctaaagtcgaaattgcattgtgtctctttaaccaacaaaaagaaagtattgtgtttaacataaatgggactctttttttttttttttttttacaagataaacgggagtcttttttttttttgacaagataaacgggagtcttagcaagcacgagaggtgtcaatgatgaaaaataaagcacatgttcaatttatgaacaagaacaaacccaataggccaatacaaggcatccatacaattgactccattgaacgcactacatgacataatgtcaggtggaacttcaagtttaattttggattaaaggctgcgcatctcactattcaccaactattatttgtctttgaaagtcaaagttgaaattcaaattttacatatatgctccttcccattttatttataataaacaattaattttttagattcataaaataattgatatatttagtctatattataatccagatgcatcaattaattattcgataaatttaaaaaatcaattttttcttataaataaaatcggagggcacgcggagagcgctaattaacatccatttaaaaacataaagatgtataaattaatgaatgagcatttttttattgctagtttcttaattgaatagccaatataaactcatacatataggttacatataggtggaggagtccagtttcaaacttcaatcaaaaagtttgataacaatttcaataatttatccagttaagctaaggatcgatcaaattatacatattaaaaaaataattgatcacattgaatccatgttatcccaataacaaattaaatttattgtctaagttatccttcataactaatatatttggtaattgaagatataaaaattaaattaaatgagatatatttcaaaaatactatgacaagttgttaagatttgtttatatttaggagggtgtattagattaggattttaaaggataatttttgtttaaaaaagtcttgaggattttaaaagacttttaaagattttgatgattttgataattttaaaagactattaaggatttcaatggatttaattcataagattttaaaggatttgaaataattgtatggatttcaagagatttcaacaggttttgtgaattttaagaaataattgaaaaaatatcacaacacactctctttcacaaaatctcaataaagactcttttatttattttactttattttttatgagagagctagagagagagagagagagagagagatagagaggaaggagagagggagagagagagagagagagagagagagagagagagagagagagagagagaggaaaagagagagtaagagagaagggagcagagagagagatagtaaaatcttaaaagaaaaaaacaatcctaagaaatctcatgttttcatgaaagatttttccatactaaaatttcactagaaaatcccacaaaatccatcaaaatctaatttattaaacaatcctttaaaatctgaatgattttgaatatcactagattttttttaagtgattaagaatcttgattgaataacacaagacttttttaaaattggaaagagtcttgattgaataccacatgattttttcatgataaaaaagtcttttaaaatcccttagaatcatgatccaatacacccccttaatttcaaaaataatgtaccaattcaacttctttttttgaaaaggatgttttttttgttacatttttttataaggatgttaatttaaattatattttattcttaagaaaatattaaataacaaacccgtttgagatggaagagaaaacaataaagtgaatgaagtcaagagcaaattcatcctaagtaaagtagtttacttttttgtctctataaaaaaaaaaaaataataaaaaaaaaaaaaacagaaaagtaaactgcttaaaggattgttatattaggggatgtgtggatgggcatgtgttcaccaatgtcaccaattctatgtcatctaagtactatccgtatctgaattagaggtgttgaaatataagccgatacaaattaaaatctcaaaccgatctgaaaaaatttaaaaccgcacaaaatcgaaaatcattagatgtgtttgtatgcacttttgtgaaaataactcggattaaaacgaattttggattgattttaaaaatggaacaaaaccacattcatattttttaatacttaccaccttcgcttcttattataagcaaacatttactttttaggttcaatgaataattatatctagtatagatcggatacatccgttattcataacttaaaaaaataaagtttttcttataataaggaccagatgtagtatttttttttattaatatattgctttagtctattatttattggttttcattttttaaaaaaaaaatactacttactagttttatttgatctatttttttatgtatcaaacttaatcgtcattgtcattccatgatactatttttttttataaatcatatatattattttgcataaaatttgttatttaaccatgtccttataattattaataaaaaaattacaatttattatttaatatctaagaatcgattcaaattaaactacattaaattggatcggattagattttttaaattagttatccaaaattgaaccacatataatattatctttaaattagatgagtttttgcgttaaaatggatacaaaacacaccacgaatactccattttaatgcatttctatactttcgctattatatcattgtttgtacgttttttaatcatgtaatttcattgttagaaattcaccgcttaagataaataagttggaaatcaagagttcaatcctcaatcttaccgtattacaatgttcctatcaactgagctatattcacggagagtggagagttgatgtaaataacacgatcggataatgtcaaaaataaaagtaacatagccattttttttaggaaataacataaccaaatttgtaaaatagtaagatttgacttagtcaataactaaataccgtaaaataaagaattagctttagccgtcaaaaaatagaaattgtaaaataaagaaagcaaaagagagtaagactatgtgcaatggggtgttgaatccaaccttcaacatgttgaatccattgcaagaggggtgttgaaaatgggggaaaaatgatgtggaggtgagagtagatgaatatattaaacatgttgaattctgacgcggggggacacgtggcgcgttgctgctggctgaagcaggcgcgtgtggtacacgcgccgacagggcgtgtgggcctaagatggaaccggagagagaaaattaaatggataaagtgaggacacgtgtctgatttcgattggctgcttggatttttatcatcttaatattttagactcaattatttcattgcaaataattttattttattttttttactaaaattcgtgatttttttctctataaatagagacttggatcatttgatttggacacagaaaaaaaaaccaagtttttcactctcttaatattattattatctttctattagcttttattttaaagttttagtgaaatggatcccactaacaatcaattcaacaccccaaatttaagttaagaaaatataaataaattatttaaataaattttgtttttacaaaaaaaataaaaaattcttaagtgtttcttcattttaatttaattataatcgataattgtaattttgtgtaattataaaaacaaaaatataaaattaaataagaataagaaataaaaggtggtggggtagaatgttgaatgaaaaaaccattggagaggtaaaaattgaatgagtgttgaattaagagagagaaaatgaaatggagtgttgaaaattgaaaaagtgggtgttgagagttgttgaatttgttttaccattataaatggtctaagaaaagcatgaaaattttctttttgagtataataataatgatgatgatgataaggtagtttggaaggtgaatgaatgattgtaataataatccaatgttggttttggacacaacattttacaaattgaacccggacgcatttggaggccaggttcattcaaaaagtgtatgattaagcctacattcatgttacactagactagaagtgaaaaaccctccaccgttccttatctttttaagtaaaagcacatgtactatagtactattcaacaattatgtataaatatgacaacatttccatctacctatcatatgtaaacaccatatcaacatttcaacaacgatatattaattaatttagacctatcacattagcaccataacatatatttgtttgcatattcttagtggtgctagctttttaaatatggctaatgttcctatgaggtttcagagagtgatggaagcatttgatgctgatgtggcactatcacgtgggaagctctgcagcgagagcagtggaagcgagcactctccggagagttcacctgatttgtctgatctcgtgttgtctttcatggaagacagcgaaagatcaggggaagaaaaagatgttgttgctcgcagcaaagattgtgatggaaagggtgctgaagatgttgaagaaattggtgaatggtgtgatagtgagaagaatgaaattttgaagggattgtttggtgggaatgaagatgttgttgatgaggatgaaaaagatgctaaagaaaggattagaagagaggttgagcttgcaattggacttcttggcagtgattcatttcctggattcaaacgttccttgatgtcttgcttaagggagaaaggttttgatgctggtgagtactaattatctcttgattatttataacttgttatattaatacctcttgatttgctccaaagctaattagctactatgagttggattcattttagttaattcgaacatttgaaaatatcgttatatgggaaaaatgattccccatgttcatttgctttaattgattagatgttttcaaatacttatacttttgggaaaagaaaattctctctacttaatactacattgaagtcatttcggattggatttagatcactaatatcaagaaatcatacatttgatgcggtcataccgatttgaatcctctaatgtttggatccaaacattagagaattatatttattgtctcacaattacgtaaaatttatttggtttataacaacttaaaaattataaacataggagtcaaatgtggtaaatattgcattggaacttgtgctgtcaacttttgaaaagtatacaaagtgttacttttattttaaaacttcatttttttagttttcttaaccagtgcctccacctctcatgtttgagtgaaaataatgtgaatacacgccgtcataagattttaaattgcctataaattgtatccaaaatataaagctagtttgagtcatttgatctcaatcaaacagtcaaaatgaagtatcacgtgaatgcataaagtgatcccctttatataatattgacttggagcacctttatctctaagctagagatataaagtacaaggagtgatatattattgcatttaaattcaagcaaaatattgacattgttactctttgtgcagggttatgcaaaaccaaatgggaaaaaaaggggaaattaacagctggggactatgagtacattgatgtcaatctttcaggaaaacgttacataatcgaagtttcccttgctgctgaattcgaaatagctcgaccaacaaaccaatattcttctttgctaaatatttttccaaagatttttgttggtaaaatggaagaactcaagagaattgtgaggctaatgtgctctgcaatcaaaggctcaatgaaaaaaatggacctacacataccaccatggaggagaaatttgtacatgcaaaccaaatggtttagttcttacaaaagaacaaccaatgcagttgcaactataaaagcatcttcacatttttcagttgagtctttttgtcctaaaaagtttatgggatttgaagctcgacatgtgaaagcatataattgtagggatgattatgtggggattaataatgggtttagggttggtcatttggctgctgctttaaattctgataactttggattgtaattatagttgcatttgttctattttgtataaaagtgctttagttaagtggttgtgttaggtactagagaacaaatttctctcatgcatagccatggaaattcttgtcagttttgggtagatatagcaaacaattgagattcattgttagcaatttttgtttgttcgtttgagataaatattgtggaaaaaatattgtacagtacatgattggtttcttggccaaagatcaatgtcaaatatccaattgttacctagcacttactatattttatggcacatgaagtacacatttgaatgattaattgtgtctactgtctaataatacgtgacatattggcctagtcctttattgagttgctgcaagggattgatggtagtttttaggtgcaacaccatatttattgagtttttaggattagattgttccaacttacaccactaaaataacacccttgtaacatctttttcatcataagattcggtcctttaaaataaatttataatatatcaatggtttatattgaggtggtgtaagttggagtaatgaatttgatggtccaaactatactttgccattgggatgtcatattgctgtcgtgtgttgtttttgcactttaatgttcacttaaaaaaaaaaaagaataatttaaattagatttaaataaatgtttagtccgcataaatatatcaagtaagagttatattttggggagaaaaaaaggaacaatgtgctatattttaacatgaatattatttatttatcatttgtgctatgtgatgtgttattttaggtgattcttcgataagtctctcagattcctcaacctctaaaataagttgattgggaataacgatgcgatctaaaaataaagaatgggttgtaactctcgataaatttgatgcaatccgaagttatgatcctataaattcaaatgacgtttgagcgaggagtctataaaacaactatcaatatttatggtaggggctcaacctagtcttaatttagtcattcttattgttagtttgactcctatttttatctgatttatattttcaattcttaactctccttcttaatgcagttgtgttgtaactatatcgatgtgagcatagctcaggataatgcataatatatgtaatgttcagggttcgaattccgaacaccacaaaaaaaaattgtgttgtaattatgccctctaacttgcgtataccttttttttttatataacctatgaatcctaaatatatgatgcggccgagatattttaataaatatttaaaagcacaatatataaatataagaaattttaatgataaagaaaatatcagttacataccctctcgaatgaaatataaacaaaaaaggtaggtgaaaagcgactaattttctaaccacgttgacttttcaactatcacttttgtttatatttcattctcgatggagtatatcctaaaaatagaacatcattgttgttaaactatatcacaaaaagaacaccattacctaatgtatactcccctcatgtcacatttataagcaaaaaactatattttttagattcattgaataattaatgtatctagtcacatttatagaccatgtacattaatcattcattgaacctaaaaaaattgttttttgtttataaatgtgaccgaaggaaatatattataattttttttatttaaaaaaaaattagaaaaatcatgtttccctttttttctaaactgaaaaacaaaataacaaaataaaatatccagtatctcgcgactatcggttcccactgttgtatcccttgcacatcccaaaagtatctgaaatgtatcagataatttttttttaataaaatttaatcaccatatactatcctagtccgtatcggagcgtatcaacatgtatcaatacataataagtatcggatatctgtatctaatcaattttaaagtatagaacttcaaaacctataacttatacttccgtttgtgctatttcaataaatttagccgtttaaaaaacttaacacaactttgtcatccaccgtcaaatattgttcatttcaccaagaaagtttcttctttattgcttccaccattagaacctacgtatagtgtcttcttggtttagcacgcaccaatttcaatgagtctgggagtcaaaccggataaacctccaacaagtatatgtagggctggacaagaacccgtggcccggcccaacccaaaaaaaaaaaaaaacgatctgagtgggttaaagcgggtcctcgggtcaaacggatcagaactacgggtatacaagggtatatatgggcggagacgggtgaaggaatttcacccgcgggtacccgaacccgcccgaagatgcgtattatattggagaagctataatacagtataaccaggactaaggctacggtgggagacctctttagtcctaccgtaggagacttgaatttttattttttttttgcaaaataaatagaagtcagccgttggattatgaacctctatgatagttagcataaaccaacaaacacctaatatatttttattttcttcctttttccccctcctctcctctcattcttggtacggagccttcttcctcacaaacaactgagatttgcagcaacctttaacccaattctacagcaacaaatcacaatatggacaagaattttggacagttgattattttgactgaattaaaaataataaaaataaaacatgattcaagctcatcactgcatttttgggtgagcatttaaagattaaattcaacatcataaaataataatttttccagaatttaatttcggatttaaagtgagaattaataatattaatacattttaatcaaaataatccccataaaattaagcccataagcttcaaacacaatcatcaatggctaccactcatagagatttatattttcaatatgtacatagtcatctactttatatctttcttccccaaactacttcttcttcctttttcacttccactcaaactcttcaatggggatttttccttcacggctttctctcttcatttgttttcaatgcatgtgagctctctgaaaattatgggtgccaaagaactacttccagtaaggaagatggtttgaaggatggtgagtgtacggtgatgtgagaaacctagagtgacctggtgaatgagggtagtttgggaaccaaaaaaattcataggaaggataccgtactaccaattgtataatgaacaattgagtttaatttttattttccaactttgcataaaaaatttaagggttaattatgtttaaggtctctataaataagcgagctttcaacattggttcctatttaaattttattaaatttttcatccccaacgtttttttcggtttgtaagtttggtccttgccgtcaaactgtgttgactaggctaacagaatgctgactcagacatgccacgtggacatttcaagtgacatggttgccatgtgataaatgacatcatatgttttaggtccctataaaattgagaccttttaagtttagtccttacttaattataatagtgtttttagtccatataaaacaattatgcactcagttttagtctctgctaaatctaaatttgtttaattatgcttaaacttttaagacttttaacaagtttttgcacaagtattaagaacatcacgaaaagtttatctgcaaaaaattatctcaaaattttttgtaactatccttaaactcttaaatttttcaatgattttttttatacaagtttagatcattacaaacggctcgtttaataaatttagaattttttaatatgaaataaattaaatatggatttttttaatagacaaaagttcaaaataaaagtaacaaaattcgggatgcaaaaataaaattttgagataattttttgtagatgaacttttcgtaatgttcttaacacttgtgcaaaaagttgttctaaatcttaaaagtttaagcataattaaacaaatttaaatttactaaggactaaaactgagtgcataatttttttatatggactaaaaacactattataattaagtaaggactaaacttaaaaggtcccaattttatagggacaaaaaacatatgatgtcatttaccacatggcagccatgtcacttgaaatgtccatgtggcatgtctgagtcagcattctgttagcttagtcagcacaatttgacggcaaggacaacttgtaaacgaaaaaaaaaacgttgaggatgaaaaatttaataaaatttaaatagggatcaatgttggaagctcgtttatttataagaactttaaacataattaacccaaaatttaaatcatcacgttctggagttttttcccaacaattattctcatgtgattatccaatatccatcaacgagaaagagcaagagggatcagtggcgtgggagagaactaatagttgaaagtaattgggtgttggatgtgtataataaacatcttgatccaagggttgattaaccttcatcctatggtttatattcatttgaaaacaaaacaaaaaaaacactaagtctcctatgttaggtgacctattaaggttgcccaccataaacggagccgtataaccagtacctaacatatgacagacatacagtgtacagtatatggttggtactccctccgtcccaaattgtatgacgttttggccatttcacacgtattaagaaatgtaattaatattgtgtggggaagagaaattatgagttgttttacaaaattatcggaaaagataaatgaaataattgaaagaagagagagtaattaatagttaagaatataataggaaaaataacattaatatttcattggtattgtaaagcgacatataatttgagacaattttttttctttccaaagcgacatacaatttgggacggaggaagtagtacacaatatgcagggactataataattaaatatgatagtattaaatagtttccttgaaaaatgtaaaccactggcagtaatgttcaaaaccgtaaactttttctcgattgatttgatgatacaagttattgccattaaaaaaaaattagtaaaaaggaaatgccaaaaaaaaccactggcctgtttgatttgctttattttttttgtcaccggctttgaaaaactgtttcagaattcagtttttataaatagctgaaaacgaaaacatctaaaagatgttttgcttctggattttctgttttaaaaaacatacggaaaagcaagaccttttattaatggcactattgtaaatatttgaaacctttccttcactattgtaaaaacccaccaaacaagtttttaaatttttcttctcaaaaatagtttttaaaagtagaactaccaaacagaatttttttatttacttctgaaaaccagttttcaaaaacagatttataaaacagttttaaaaaactaaaaagcaaaactgaaccccttaatatctctctctctcttttatttacatttattatttcctttgaatttatttttggctttgagagtcacagtacaacaagcatcacattctcttcttgcttcatgaaatgcgccgctaacaaacaagctaaatcacaaaatggaactggatttgtctttttttcctaccgaattgctttgtttgagatcttgttcccttgtaccaatggaagttatacaacaacaaatagatctggattatttattcaaccttctccttcttctttccttcttctcgtcttctttttcatatttttttattcatttgtttcatgtttttttaagttgtatacaagttattttcttctcttgttgcttgcttcactagatcagtttttttttttttttttttaagtttgacattgtatccgttttaacccgcccgtaaaacccgcaacctgattgacccgtgacccgtctaatccgcaagggaaaatgggcagcattgggtctgtatatgtaacccgcgggttgaggcggaattgggttttcagcccgaacccgcctgatgtcctccatgaaagaggaacgaagttggtgaatcttcttgtctattagtacctttttctactttaaagtctcctagtggacacccgttcactaacattgacaaattcctaactaaaatacaatatcacatccactctattcatccatttatctcaaaaacctttttttttttagcacgaactccaaataatcccaatcaacaaagtcgtaagctctctcactctaccttcaagagcaaactcctagagggtgccttctatggttgatgaagttgaggttgaaaatgatgaagaagtggaaggaagaagaagaaatgaaaacaaggtttttaatttgaaattttgttaatccttgtttttgaatttcttccctctataagaggcatcatgtcagccatcgttaacaaatatgcatttttgtaacaatgatgacagcaaggaccaaaaacaaaaagtttataacatacgacgttgttttaaaacaaaaaaagatataaagacgaaaacaaaaaaatacgctaacttacaaggacaatttacatatttaaactctattaataataataatattcttaaataacttcttttatcaaaaaacccaggaaaaagcaacattaatcataatcaacttcaattctctgagaagatgaagattcttgatttttaggggtttgaactttaaagaaacataatgaatatacggaaaaataatgttgattttcttggggtctataattcattaaaaataaaataaattattattaataatcttatgttgtatatcaactcatcattattaataagcctcacatccgttttgaaaaatgttaactagtgcctcaggcaagttaacaagaccctttaaaaaataaaagaaccagagaagaacgtgatacatcagaatagagacaagagagaattcacattgaaacaatggtgtattttgatattctaaaatcgagaggtattttcatattttcgtaggagatgagaggcatggaggatagtttaggaaaagaactcaattcaaagtcaaaaattgcgactccatccattccaaatataaatgtccagattgtgtgattgcatcaaatatgtgcttttctattgggaatttagatccacaaaattatactgttatttatgttcactgtaatactatcataaatggcgttcagctaaagtttatttgcatttgtgtaagatttatttttattaattaactgtttttgcaactaacaaacgcaatctttttgaaggaccaacagtgcattagcaggcaaacaacagcctctactaaatatatataaatcacaagcctgtacaaattttcaactcttttgatgctagtgaaaagcaagagttagaaggagattgtgtaagtttacggtaatttttcataaagagagatgttggttatccaaaaaaaacgcatcgcaattcaccacaacaatgttgattcaccaatactctcatccgcctctcaagatcattaatgagagatgtgaaatagaagtctgtaagtatctaatggaagtctggctccaaaattaagacaacaaacttaaccactcatatcctcatatatatatatatatcttttctaaatgcaatcgattgataaagcataaaaacaatccacaataacgtgtatttatctgttggcaacgttagacgcaagatgctggaaaatcaaagcaaattacttggaaagctacccttttgaaaaaacaaaagtgttcagtataagagaaaggtcttcgaaataagccaatcgcatgcttatttctttcttcttcctttctcaaattaaaaccaaaaaaatggatcgaacagaaaataaaattgaaagaaacttaaaaaaaatgtgagaaactcggtaagcagatttatcgacagagtcacaataaccggccagtaagattcttcgatttaatatagctacagccacaatggcacattcaagattaagggtattggtttttttgttaccacccttctctcggtacaatgtagcatagttacaaattaacataaataaaaaacagagaaactgaacacacaatacagaattataggattagattcataagaaagcgaagttgctattgagaaatttggaaaattgaaagtaacgaaaatgaaaaatttgctgcgacttcagatctgtgttagctatcagccgtaaaaccctaaacctaaggaagatatgagtctgaagtgagggctggtgcgtatttataatcgaaatgcagggctgggcctaggaaacgtatcatggccacttattttttactgaaccgatggatataaaggaggtgtagaaataaaacttaatcaaattgttaatttggtaattatacgctccagtgttgtataatgtggtctatcatgtgcccttatttatcaccgttagatgtcttattattttattcagtgattgatatttcaattttttaacaagatccttttattgtgttttggttctacgatagaaataaataaaagtgattttttttaaaaggatatgaaaagaatatgaataagttgtagttaaaaataatataacaaaaagtgaattatctttcgacttttgagctaagcccttaggattttcgcaatgaaagttgtatgcgtgagatgaaaagttggtaggagagagtaaagttttacttcacaatatttttggcaaagaggatccgtgataactgcaattagatcatgttgttgattatatgattagcagcgcgtatcatttgctagctaatcctgcacctcaatttcattcatttgtttcggatttaatttggtataaggatgtttctctataggtatctatatttgtttggcgtttcttcacagtcgactttctacttagggtagctttagacgaggcatcactagtcatgaatcacaaaaacccacttagattgacatggtggtgttggctaagaccttgaaacgtgcaactcctcaaggtcacgagtttggttcttcctcaaatttggtgggctagtttaaattattaaaaaaaatcatgaatcacaactttgtttgagtggttgtggtgtccttgaattggaaaatcatttgtttttggaatgtaatcttttcagcgttatttgataccttgtttgtaattggattgagatatcttcggttgatcctttatgtatttcagatcactttgttcaattttgtcgtgcatttttggagttcaacgatctataatgcacgtaatttggttctttatgtctgagtaatttggaaatagtggaatgatataatttttgaaaataataaagactcgattcatcgtctcttggacaaaactacgttactctcttgttggtgaccaaaggcaaaataaattaatattgcttttggttatcatagatggtggcttcatccttgttttttttttttgtttgggctttggctaatagttctctttccttttcactcttttcattttcgacttgcaaccttttgtaaaacattggtattcttctccgatagatctcatgctaggtataatgcttttttgttggtaatatatttcatttgccttaaaaaaaaggagaatttatataggtttaagtagtatgcttgatgttttaggtttaaacttcactgtctcatttgcacacaagagaatctcaactatcactgaaccaaacacgtaagttaatttttttggagctcttgtagttaaaaattgaattatgtattatggttttcgtaaaagatcaaattatttgctttagatccttcaacatgtcataaatacacatgatagtttatgggaatagtccacactttgataatcactctcaaactttaaacaattatatcatactgaacttctcctaatacaatatggtacaaagacaaactaagtgggagttggtcgacatgaaattgtatcgatgcataagacacatttaataagtggctttttacacatttaagcggcttatattcacctttataattgaagaccaaaaatgaattgattgatgcaagttacgtcaataatatgcacgatcttcttggagcataacatgagaaatttacagtttagtgtgtcagacatataatgcctataaattggatgaccttctcaaaagtttctcatatgttttgaaaaatgataacaaaagagttataaaggggggcatactaaacaatcttatatggagtctaaagtcagggccttgtccgccttgccaattatacgaccatatcctgattatataatgtaggttaatcatatgatatcatataatttgattccaccctaaatgttattatcgagtttggttacttagttaagagtgcaatatacacattcattttgtttacaaaatctttcatgcgaagtttaatttgagacaaactaatatactttttgatagtcttcttaaatcatgcatatttcacgttaatctttgaatatacaacaacgagaaacgctataaacatttttttattggacgaaatcaatatagatctcatattttaaaaacaaatcttatataaagtggctagaggggtgtaaatttacaataatacaaagagtcttaagagcataaatatcagttgacttagatgatgtctttacttctctcaagaactttactaccgtggtttactgatatttgagaaacaaagacaccctatgctataccacggtggtactgcaatgcaacagtgcctttcactttttttttttgagggaggtgcctttcactttttgcaatgccaacatcaatgtttttatattaaaactcccaacttttcaccttttaactatattttattaatttaaaacacaaagttggtgtatattgtaagattaatggaacccattttaaaattttagaagagtggtatagatatttaaaaaatgtagttgagtggtttaaataattgagaattgtgaaataatataaaatattagatgagacccatttataccacccatatgagtgagatggatgtaggtgctctaagggtgtgtttggatcaacaaataggttaaggaaagcacgcacaaggaaaagaaagtttctcaataaacgtgttttcacagtgtttccatggtttggttcaatgtcacaaaggaaagaaagtgtgattttggtagaacccacgatctaaaacttttctttcccacaaagtgcagaaacatgagggaatgcataaattacaaaataaccattgcattgaaacaaattattatcaacgttatttttttctttaattaatatgcttttctttaatttataatataatataatatacttataaattacttccaccaaaaaaaaagtatttataaattacttgttttaaattttaagacggtacttatatatatatatatatatatatatatatatatatatatatactttaaatattatatctattatgtagggatttttatgtcaatttactaactatataattatcttttcctttcattttcttcttactatcactcaaaccaaacaactacacaaatcatctcactttctacccactttctatccaatttcattccttttacattctttcctattactttctttcctttccctttctcttctaatccaaacacactctaagagagtggagtaacaaacaacttgtggacggcgctgattggatacaacataaccaatagctaatgcgagccattggataactcatcataaaacctggaaactaaactataaaaacaagcaaaacacactataatttatagagagagaaataaaagcgtttttgcatagagaactcaaaaacgaaatatcaattttttttttttgtttaaaagacaaaaccaaaatttttgtcattgttagaggtggatcgcagtaatcggaaggcagatctgtttagttagttaagatggaaggagttggggcccgactggggcggtcctcgacccgttacggaccggcaacggttttcaccggaccggttagaaaatggaagaagaaatgggttcatgttttaccttcatcttcttcatctaattccaacaacaacaacgcgaatcaaaatcacgctaacggtagtaacggtaataacggttctcatcttcttctttttaagtggactcctattactcaaagccagaacaacgccaacggtaatggaaatggaagtgttaaggatgcggcggtggagccggtggaagagccaccgcggcgcaagtttaaatatgttccggtaattttcaattacaatgcactccttttcgttatttatctttttattggatttggatattatggcgtagtttgattcatgtagccgaggtagctagtgggttaaagcgtggctgtttttgttcgggttttacgagtttagtttagtttagggttctataggatgaattcatagggttttagtgttgagaatgggaattggaattgatattttatggatgctaatattaatatatatgtttggttccacgtttggagcagcaagaattgattctggagttgtaaaattgacattgacttgtttgtgtttatgttaattgtttgcatttttatggaattgataattaactgaccaagattgttgttcatggtatttattagtgatgattcttgtagatttacttcatagaattatttattttttcatttctaataatgattaaaaaaatggtgaaaaggaagatacatagtgtgaggaaacttaattctgtttagatttagattattttttatttcaagtgttcggtaagatactaaaatgtctttttggcggttttccaaatgacaaaagtaaaggctcaaattagtttttggttaactttgcggaactagggttataagttggtttttagttagatgaaactagaatcagaacaagatttctgattctttgcttaagaaatgaaattcactctagcatgaatgtaggagacggaaatatggatgccttgtggtgagcgagcggtgtctgtcgagtgtcatggtgttgagataatagtaactgatgagaattcctataaaaaattgtggtatcttacgtgaggattggatttggaaaataatctgtaaatatgataaatgtaagttggcaacttgtatgatgaataaaagaacaactttagtgctgatagttacatgattcagatatcagaataagaactctatgaaaaagtaccacagaatgagaatccaagagtatcggaaggagatagggattcttttttgtagaaatggaaaggaaacataacattacacaatcgtttgtggccgtagtcagagtcttatggaaaaagaggatctatttggcaaagatactgatatctgccaaattattttacagtaatttgttattgcctatattcgcgcgtagataccaatttctccattgtgtcggtatatggttatttattaatacatcttttggatggattactgatcatccgggaaaagctttttttgtcggttatatttttatattaggaaatattgctttatagtatagacaacataattgtacaattaaccgtaaaccctcaatttagtcctcaaatgtcacgaagttgattggtaaacaataagttctgggctatttaatgcctctcagggaacaaactgatgttcgcaaaatcttgaggatcagaatgggggttctggatacttttaattgtcctgatgcatgtgagttagttagatctaaaaatagtggttagccgctgcatcactgataggatttcaaattgaggtataggcacgatggtgctgtcaaatgattttgtttttaaacaatggacggatgatgatcatatacctagccctaacactttaattatcttttaatgaaattgataattaactgaccaagattgttgtacctggtattttcagttatgattcccgtagatttacctcatagaatgtgttttatctattagtgattaaaaagagtggcaaaaatgaagataagaagtgtgatgaatcttaattttattttgagaattttttattagtgttcgttaagatacaaaaatgcctttttgtcggctttgtgaatggcagaagtataggtacaaattagtttttggttaactttgtggtactacttgggctataagttggtctaagttttgatgaaactagaatcaggaacaagatttatgattttttgtttaagaaatggaattcacgatagcatgaatgtaccaaaatgaaaatatagatgccatgtggtgaaaggtgttgagtgctgtggttacgaaataaagttaactaatgagaactcatacaaaaaatggtggtagtttttgtgaggataggatttggaaaaacatctacaaaaaatgcaggtttgaaacttttttgatgaataagataacaactgtaatgctgaaagtgacatgattcaaatatcagaataagaactctacaaaaaagtaccaaagaatgagaatcagagagtgttggagggagacaaggattattttggtggaaatggaaaggaaggatatctttacatagtgatttgtggctgtagacagagtctaatggaaaaagaggatctattcagatactgatatctgccaaattatttttcaataatttgttattgtctttattttgtgtggagatactgatctctccgttgtgttggtatatggttatttgtttaatacatctttagcatggattgctgaccatcttgggtaaactttttttgtctgttctgttgttatatcaggcaatattgtattgtagtgataagacaaacataaccgtacaattaactgtaaaccctcaatttagtcaattagcaatatcaattaagtcttcaaatgtcatgaatttgattggtaaacaataattttttggctattttaatgttaaatggcagtaatcgaggaccaattgatgttctaaaagtcttgagggtgttctgtctacttttaattgtcccgatgcacttgcgtttgttagatctaaatatagtggtttgcctttgcatctctgataggatttaaaattgagttattgacacaatgatgctgtcataattttgttttctaaaaaatagtcggacaatgaccatatacctaaccctttggaattgttgtgcagtactgttgttgatttttacctttttcgacagtgctcggcatataccaagaccgaacataatggaatattctgttttgttctctttggtcaactcctcagtaatccatttagtggaatcaaaacgtaccacttttaaacttcatttcccattctaatttacgtgtaaagcgtcaaacatatatgtttccatgtcttccttccatctaccaagaaaaaacatatatgtcccatccttgtttaacacacttatcaaacactacctcagtgttcccttgggaaaataacacttatgttggaaatgtcttgtgcaaccttcaacggtctttttttatgtacccctcaaacacatttgttgatcagagctatagtcatccatagttgtcaatttttgaatcatataactttacaatactaggtatcttaattgagttaactcaaaaagttgcttctgtggtagacttggataggctcgtatagagtcacaagtatgagtacaatacaatgacagtggtagtgttttttaaaaaacccaatcattaacctcacagttgtagcacttgtctccatagatggatctagcatctcaacatatgaactcatctataaacaaggggcggtgttgtactgagtctgaacctgacagctcccagcatatctttgcaaggctgcaatgaatccaacaggtctacaaggtaagtgaacccaactctgatgagctttatccagctccaacgagggggcccagtcccaccatccttcactcttcagtaattttgtactccgtgatctatttggtctctgatctattctgttatgcctggaacttgaagtcaacttcgttacactttcacattttcaccacgataaatcatttcataatttctcgttttgatgctgataatctgggtggatgatcataaattaatatatcatgttcaatagtatggggattgggaaggcaatgacatggttgtaatctggactatctgatgatgtaggttgctgtactagaggagcagaaaaacgaggctaccgaagatgaggttgccgaaaaggttgaagatgaatctaatccaatagaggctgattcaagtgcagcagaaccaaccagcaagaatgagactttggatgaaaaacctgatataaatgatgtcccaatggaagaaagcgaggtaaacccttgcttgcctcttcatttacttcatgtttatctacataccaataccatgaatttttgtagaatgtaataactgcagctcacatccaactcaatggcgacttagattgataaaagggaactatttttgtagttagaatcaggacatgcttattttgagagaagtttctatcttttccatggaaataatcatttgtttctttttaaaagtttgaatggtgggaaatgatgaaaagacactaaaatgttgctttcaccatttcttggacacatttaaaatcaagaataaattaaagataaggagatattcttgtgtttattaatgacaacatatgttttcttaaactaaacggttcttaagaaaatttatgctgtcattcctaaaaaaacttttgtcagtaatcatcacagcacatcttcttttgacgactgtttgaagattggagggaatagtgattgtcctcctcaactttcttactgtattctgtctgatgcattaggggccatcatactgagacgttagggcccatttatttatcgtgtttctgtttttaatatcacttcaactaatcaatataagagaatatagatccaacatttttcacaccattttcttttacgatatttgaaaatagatctagatctagcattttcagtttttttttttcgaaatgtcaaaatattgttttcaatgcagtcttcctagcttgccttccatcttctctctatcacaattgtttaatacaataatacataattcttccatctccttgttagcaatttaaaataaacataaattttagaaaattgaaacaggaaatgttttccaaaagtaatcagggcatagtttttcctttttctctgttattgtttcaacttgctcaataaaaaaattaaatggctcgtctgtcattttagactttggacaagttttatggttcacacaagcattacacaatttgaaaattgtagttttgaaattattttcatgtaaatagtaatctacatgtctcaaatttgtcgcacctgtgaccaactcaattgaagttcacagcagtcactagcgagctatttgttctaatttccttttattacctttattatttataatgaggactttatatttcagcctcaggacaaaaatcaggtagtacgccaagatctcaatgaaagtttggatttgagtttgggtttgacatcacatgatgaagatcaagattctgattcaaaaactaatcaacctacagatggtcagtaactcaaggcgaactcaagtggtactgagacatgaacagggtggcctcatgattctaagtgattcagtctttatattggcctgcagaagtgaattttcactctggctagatattgactccctagctgttttaggttggattgttgtagtggcatattggcatatgcccattgtatgttttattattcttcaatgtccttgctggtctgattagaatatgtaatgcaggaacatatgatatacattttccaatcattgagcagaattttttcccagatacttatttcgacagaatattatgggacttggaagctacaatcatgcgtagctgaaattgattgtttttttaaaagaaaatcaaacaacctaacttatgaaattgtgctattggtgccttataaccatattatagttgaaccttctaccaagattacattgtgttagagatctaatggtatctgtatactacttaagcattgaaatgtaacaggcactttacacaattactggttttgacttgtttcggtaatccacactgtaatgtttcatcatccactgattttatttagggtttaaacgaagagattctcttgctccatttctattttagtgtggtgaaaatagtgaagggaggtaggtgggacttcgtttgtttagcaaagttttaaggtgggtttggtgtgaagaatttgatagtatttaataatttcttttacccaattggatttggcgatgtttacgtgataaagatgctatttggcatgacatgcgttcttttaagtatggggatggggatttgaataaatggggttgataaaaaaagctctaattttgtgtatgtggggagttggtaattgtggtacgatgataggtggttttgctcagattgttttcaagttagtattgatgagtgttttacggtgaataatcatttgctttctctttctctttctcttaagagtaatcaccatttggatccaagtattgaaaaatgtttggaggtgtgacattccttcaaaagcacaggtcttaacttgataattattgcaaagacttacaacaagggatgagttactgctgttaaataataataataataataataaaaaagggttgagttacggcaaaagggaggttttaaaatctattcattattttttcccaaaatgatatttcaaatatatgttcaacatgaaaattacaatccaggtatcaagctccaagtacacaataacttgatattttcaaattctccgtcatcattctttttctacagccttctccgacgaataatcttgcctgggaagtagataatgagtaagatacatatcagcatggagatacaaaatagtgtcaaaatcaatctttgcaacatcaccaaaatatatcatggctatcacacaaggttaaactacataaaagagagcttttcaattgacattgtaattgtgtattttgtccttctacgctgctctttaggattttcaagaaaaatgtcaaaatttgattttatgataaattcaatcaaaccctgattttatgataaatttaatcaaacccactaaagaaaataaacttgattaaaaatagtgttttgagtttcccatttttgggcccgggaagggccaaactaacatatcatcaacaacgtacttttgggcccatgaacattgaatcataaagtagcaaatggcaatctcacaaagacttaatataaacattgataaatatcacttaccgcaatttgcgtgacagttggtataggccagttcctgccattgcaacgttcacggcaaaaaggttccaatttttctgcaacatcgtaagtagttgcaactaacatgtcaggattatggaaaattgtatagccacagaaactaagaaaattacactttcaataatatgcagaaagtaatccatttagtagttctcaattttcctactcttattttctccagccattatgatcagtgaaccaaagttcatttagttaattgtcttgtaacaaagtagtaacaaaatgttgctgtgttaaccgaggttaaaattcttgtatactctattttaacaccaccatagcataaggttttaaggtttttatttcttggcaggcacacatatatactgagagagctgattggtgtacgataaaaattcagataatgcaaacaacaatagggattaaagaaaactattatttatcacatggcttaactaagcaacaaaaaaacaggaatccaagttttgattccgcattgcatattacatgtaccatgttatagtctaattgatgttacagaatcttacaagttgagtatgtgatactcttgcaagagagataatgcaactacaatcccgaattatgcacacatcacttatagtgatgaaggtaataaagtgaaactatataaaactttgcttgcacagaaggtccacaaatgatcttcagaccactagtatttcaatatttcatgaacaagttaaaggtaatctgcctttgaaaaaaacaaaacaaaacttactggagtgattactgtgctgtaacgtgaccagataagtccagtagctgtaacagctgtgtcaaaaagaaaaggaatcagcaatgcatatctgtaaataaaacccaatagatgagaatatatactaaaactgcttgcaacttgaatgctcagcattccattattcattcacttgggcatgcctgctcctttcaaagaattcagaaatacacattttcaaaaggatgagcgaaatcagtacatgcatgcgcatacctatttgttgaggataggaaagcttctcaggtggttttgcaaaatcagcaatattggcaatgctgataccccacttaaatgtaggggcccaaaagtgaactgcacaaaataagtggtcaatttatacatcgattacagcttcacaatgtcaatattaacacttgaaaacaacattggtgactggaaagagacaacagtttgaatcaggtgaaaatgtaatctatttggtacttgtacttaaagtaatatgtgtgaagaacatatatacatcttcatcaactttttgagtaaaccaccaaatccgtccctaactttgtagtgtgttctcaaataggtcctcgagattatgattatttcaaaaatgtctccgcatctgtcaaatttaactcatagtagtctttatagtcaggatccaaattcaatagtaatgttaggaaccaaattgatagttcgtggttaaatatagagaccaaattgctaaaacgtgggtaaaacatagggatttaattggtaaaaatggttaaatatagggaccaacttggcattttaatagaatcaaatgttggaatatcaaacaaagatgaccaatctcatagatacaaagttatggcatattatagaaatacaataaatgtgtacttgtatgatcgatggatcattcaaggctactgagctatcatgagagaagccatgagagacaaacaagggttgagagctcttctctcccttcctgatacgatcaaagtgtatgagtgtaatctgacctaatgggatagtcaatatatataagcaccaaagagagaggagattatatatcctgactgtcccattaggtcagagctctcaaccctagtttgtctctcatggcttctctcatgatagctcaatagcgctgaatgttcatacaaagtacacatttactgtatttctataatatgccataatcttaggacctctttgagagcgtcctacaaagtcagagactaacttattggtttattattaacttatatcacaggccatcacaatacacataacaagcaaaataactgtaaaattgaagaatgaaccatttttgcaaccctaaaaaagacccattttttacaaagcaatcaaaagtcaaattaagttaactgaaaaaaaaaatcttagcttacgaattaaaaattgacttgtaatgaatcaataatcagagatctacttgtgcaaaataaataaataatcagagatctaaatccaataactgttatacatatataagatcggaagtggcgtaacaaagggaaaacgacgaattgattaacaaaaacatgaaaggataagaaaagaatgaaaaaacgaagtaactcactggttttgggaccagctggatgattccaaaaagcttgaagctttgaagtcgccatgaaatgaatcgttcttcttctcttcgattgaaaggggaattcgatttgggttcagctaccagtctaccaccacttacaaattatattatttgctttttttttccgtaaaaaatattatttgttttcgtaatttttttaggtacaattcttatttatttattactattatttaggacaatacaattgttttcttaattttttttaggcacaattcttatttaggcgagtttataatggttttgccatttcatctatctatcacaaaaaaaaaaaaagagaatacaaggaaaatagtagcatgttcattctaatcatttgacatctcatttttattatttgatagttttttttatcaagaaatcctttttagaggagaaataaaagaattgcgaagagattaaagtagttttataataaaaaatctcttttatataattgtatataaacaaacaaataacttctttttttaaaaagtagtttttatttcgttgaacaaacacgaaatagtttagattatttttaaataactaaaaaataatcgtcaaattatcaaatgcttaagtcatttttatcgtaatatgtaacaaacgaacataattagaccgtttattataacttttatatgaaaaatatatcatttataaaacttttataagaaaatatatatttttaaaataaaaaaaaatcagtttaaaaagtatctaatccttttgttacaactttaaaaaaaataaatcaaaatctaaaaacaatttgaaatgaaaaactattaagagtagttttccaatttatagatttttttagagaagagagaaaataagaatcaaaagatggatgtaattttgtaataaaaaaaattccttttatttatccaaacaaacataattggttttttaagaagtatttttattacgataaacacaaaattgattatgtattttttttataattctctgaaaaataatctcaaaattattgaatttttttgtttgttgagaaaacaaaattgaatgtcaatattacttcttttttaatccataacaaacaaaccctatatttaatctcatttactaaaaatatgttttatgatatcgtgtaagaggattcaaggacaactactcctcctcgattagctatcattttatttggggatatctctagatgttaaggcagcgatttacttgtgatttgtgaacagagtaattcacataatacattgatataagtatttatatccttgatttgtaacttgtataacaagccaactaactaactcttaacaaacatagtagttactagttactaactaataactacttctaaccacttattagttgaatcatacgacaacacattcgttcaatgtatgaattgtatgtgtcccacttaattggggcatcatactccaaataatttattttcaaattcttcgtcgtcattcttttgctacgatttcctttgaggaatgatcatgcctgagaagtagatcacaagttaatatacgtatcaccaccatggaggtacaaaacagtgccaaaatccataacacaaaaatatatcaatggctacacacaaggttaatctacaaaagaaggaagtttctcacttgacattgtaattgtatattgtcaggaaaaatgtcaaaatatgattttatgataaattcaatccaatcctctcaaagaaaataaactttataattactgattaaaaatagtgttttgatgttccaaatatttgttggcctagtaaaggccaacctaacctcatcaacaacatatttttcagcccatgaacatcgaataacaaagtagcaaaaagcaatctcacaaaaatttaacagaaatattgataagtatcacttactgatacttgcgcgagatttggtataaggcagttcctgccattgtaaggtttacgcaaacaaggttccaatttttctgcaaaattggaagtagttgccactaacatatcaggaacatgggaaattatatcgcgtaaacaaaagttcatttagtaatagttttgcaacaaaataatactctattttaacactaacttcaacataaggttttaattttttggcaggcaaacatgtatacaatgagagagctgattagtaacgatcgaaaatcagataatgtaaacaaaagtaaggattaagtctaagaaaaaccaaagattatcaagaaaaagataaagccaaaaattatttactgcattgtatattacacgtaccgtgttatagtctaattgatgttataaaatcttacaagttgagtatgtcatactcttgcaagagagataatgcaacgataaatcagaatcatgcacacatactctgtagatgatcctcaatccactaaattttcaatatttcacgaactatttaaaagtaatctgtctttgaaaatataaaacttaccggaatgatttgagtgctgtaacgcgcccaaataaatccgctacccatgacagctgtgttgaaaacaaaaggaatcagcaacgcatagatcgaaataaaacccaatacaagagaatatatgttaaaagctgcttgcaacttgaatactcagcattccgtaaattcattcacttgcacatgtctgttcctttcagagaatccacaaatacacatttttcacaagggtgaatgaagagtattagagatgtataagttagttacatattactatattagttagattagttggtccaattgtacccaacgtgaatatctctgacgctatgtacttcccaatttcaatccctttcagtgatgaatatccatcatatagccaattggactaactaactcctaactaatatttaactaacttttacatctctaataacgagcaaaaaggtgtgcaggtgtgtgtgacagagagaagttcgcaatagataacactacgtgcagttctatatgcatgcacatacttatttggtgaggataggaaatcttctcagttggttttgtaaaatcagcaacattggcaacggtgataccccacttaaatgcaggtgcccaaaagtgaactgcacaacaaaagtggtcaattaatggattgatacaggtttgacaataacaatgctaacacttgacaacaacactggtgactggaaacaaatacgaaacaacaatttgaatcaggtaaaaagaatgtaatctatttggtatttgaacttaaagcaatagttaatcacggccgttaattcaacataatcacacagttagtgtcatgcggtaaaatgatatcagtcaaagatccagatcaaacggctgggagttgtgtgacgctgttacattagggaatccaattccgacataacatacacacatacatgtgtattagtgtgtgcagaacatttacatcaagtgttatccaaaaaataaagtatggtttattataatttaaaataggcaggacaccaaaacagggttgatttttgccttcctcaccccgatgtttttttttaagggtttgtttaagtgttactaatttactagaggtaaatataattttgatacaagggagacgggtatcaatgtcccagttacctgtcctgctcctgtcttttgaaaccagggaaaactaaactcaattccagtcaattttaaggttcggggtttcctcgtcaaactcaaatccataggtatgaattatattgccatgtctaactcatataattttcacgtgttagcttaatacattatgctactttcactcacacatcaaaatccaacaatttctttttgtgatctttggtaataataagtacgtccaagtttcataaagcatgcatcaggcatttgaataaaaggaaaaagcgaagtaactcactggttttggggccaattggatgattccaaaaggcttggagctttgaagacaccatgaaatgaattcttcttcttcttcttcttcttcttcttcttcttctcttcgattgaaaccaaattggttttggaataaagcttcttctgccaccacttgtgatttatactattttatattttactattgtcaaaaagatttcaatcatctatcttcggaaggataggaacttcctccatatcagcaaattaatttgcgacacgtggatatataccaggtcaacatactttattttgacggacaaggatgatctgcctttttggagtgtagtgactttaggtaggaaatcagaaaaaataagatcatagataatgatatttaagacaatgaaaaagacgcgctaagataaaacaacgagaattgacacatatccattgattgtgtcatattaatttatcaaaatgtttctcggtaactgctgaattatggaatcggctgtagttaattatcgaggaaaacttcggcagtaaactatcgaggaaaactgaaaacttcggcagttaattgctgaggaaactcaaaccttatttttttttttgacaaaaactataaattgtcattaataatatttattgattttgaatgttttaatcattattttgtacgtttcaaacaattgcagtattatacttatgcgtatatagcttaataagaatagcatttaaatcagtgtaaaaattaagcatttgaatattgttttgcacattacttaaatacaaataataataataaaaatcgtttaaataattgtgcacaacgatttttttgaaggtataatatgttgttatattttctacattaaaataattgtgcacaaagattttttttttatttaagtaatttgcaaaacaatattcaaatgcttaatttttacacttattgaaatgttttttgaggacttaaatgctctttatttttttggtcaagatttaaattatactcttttatatatatatatatatatatatgcataagtataatacttgcaattgtttgaaacgtacaaaataatgattgaaacattcaaaatcaataaatattattaataacaatttatagtttttgtcaaaaaaaaatcaggtttgagtttcctcaacaattaactgccgaagttttcagtttttctcggtagtttactgccgaaattttcctcggtagttaactgcagccggttccataattcgacagttaactgctgaaggacatttcgataaattactaatgtggtgcagccttatgtaatgtgtcaacaacaattctctaaaacaattcacctcattattattataggaattaaaggatatgcacggcaaatcatgtttccacattcttccaatcaaaatctataatattgttttctcatgttatacttaacttattgtattccaattcacctcattatttgataaaatgagcgttttagttatttgattcacatatgtcaatgcataacactacaaaaaagtaacactataaaaaaatatatcaatgtataattctgatctttaatatatttaattatttatcaataaaaatcataaaaaatttaacatctaaaaaatatttatcgaaataaatccaacaaacatcttatatgttattatttgtttaaatattaatagaaaaacatgcaaatgtatgttatatgaataatgtacatagtcaaattaggtcaatcattttggatggaggaaatatttcttttgcaaactatttagatcattgaaattaaaataattaatcattattttaaaaaatgtataaaattcaaatgaaacacctaaaatgagtttgctcctctcaagatctcaaattcgatttcctctgatgtcaatttcggtgggagtagtggttgtttgtattggcgttaaatgtgttgtaaacgcgcgtttcgacggatgctacaatttgtatcttctatattatcacatcccaaagctctcaaatccacgacaacggttatttgtttaaatattagtagaaaaacggcgacacatgtttttttcctaaacacttcagagtttggagggaaggaaaggaaaagcttggaaaaaaaaaggaaggagtaagtgaaagaaatagatgatattggaaggatgggctttggagacttaatttttttcaaaatataaaaccctcctcatttgagggaactaaaaattgtatcggaggatggttttggggggtttgggagagtttatatgaattattcaaatttaatatatattgtcataatattattaaaattaaaaatatattaatcataaacattaatttatcactctttaaaaaaatactttttgaaaaaatgtgagatgacactggaaggatggcctttggggacttaattttcttcaaaatataaaaccctcctcatttggggaactgaaaaattgtatcggaggaggattttggggggtttgggagagtttatatgaattcttcaaatttaatatatgttgtcataatattattaaaattaaaaatatattaatcataagcattaatttatcactctttaaaaagatgctctctttgaaaaaatgtgacagatttatcaaattttccccatatttctctcccatcaaaatattttcttcccctcccctccaaactcccgagcaaagccatagtaaaaaaacattaaacacacattcacaacaaccacttataaaacacttgccggataaagaaggatttaatctgctaagaaaggtcataattaagaaagaacatagaaattagaaatctcttgttaaaggtgttggatcatccagttctctgagatgaatattgtattatgaaatgttaggggggattgtcaactttgacccaaggtttgtcctccataatctgtgtgtctattataaaccaaaatttatttttcggctgagccatggaaggttggaataattttacatattgcaggttaacaaagtctaagtctgatcatcatcctattctattgtatatgactaatgcaatcttatgcttgtgcttcataagaagttacagctccgaaatgtggctttgaaaatttggattaaggactttttttgaagatattaaggctatcagagaggttgattgtgtttaggagctcattgataaatttggcgaaaatgagcggttaatgcacatagtatagctcatgatttgttacaacaagctttacattttaaggatcaataatattgcaaagaaaaatccacattgagttgattccaagaggggggtaagaatacagcttatcaccaaagaatggtacacttctaaacaaatgactatatatgcttagacatgctgataacatcttgcagcatcctgaagacattgagacatgtccatagcatttatctgacatttttgctacccctaatgtcagggctatgaataatagtggtagattctattccaactatggtgactaaaatgaaaaatgctaaacttacctaagccttcatttgtagaaatcaaagatgcagttttttctttaaagcagaagagtgatattggtctttatatatggatttggtggcactttttcatcataattttgaagatattattggaaatgatgtgtataatgcaactaaggaaaggttggattcttcctaatcttaattctaatttagtagttttaattcctaaattttctggaaccattgccttggcaaacttccaatacaaaataatttttaattttttggaagacaaacttcttgttgtagcaggctagcagccaatctcatatatgataatcaaacaagatttgttaaggatatgaacaacatggtgtataggaagtcttttgaaggtattattgttctacaaattgaaatatctaaagtttttgatattttgaagtagtttggttttaacaagttttgcacttggattcatgttattcttcaataggttaagttgtattttttttttataaatgggaagaactgtaggatttgttttgttgcaaaagaggtgttagacaaagagactttctttcacctcttttattttgtgacagacatttgcttggtcacaactacaaataatttcatttgggcgcacagacacgcaaatggggaaaaggtaaaagaagaaacactttaatactatgttattttaaataattaaattgataagtacttttatgtttgtgcacctaaatgtattttatttgtggttgttcccatataattgtttctctgattttgtttggttgaggaagtgcttggtagagctttgacaatgaaagctaattaatcagttttttatttgtaagagctaatcaggggtttatcaagccaatgtttagtcttagggtgcaccattcctactcattctctcaatgatgatgatataatgatattttgtcaaggaaacaagaagtatctgaactctataatggttctgttaaggtagtatggtgaaacatttggggggcacattagttctctaaattttactcatgctcttggccattatccaagaaaactgatactgctaaaactttgggatttgtagcaggtgaattaccctttaaatatcttggtgttttaattttttagggtaagcctaaaccaatccatctcataacatatgtacagtatatattgttactaattccatattttaacccttccttctaaatatgacagttagcaaatttaaaccactcaatagagtcccaaaattggttggactccaagcatcatgggatagacatttttggtttgtctctacaccttatcatattattactcaatcgaaattaatctcacactcttaatcttaaattgaagttttacaactaggatggttttagagcatggacgcaagatcacaacactagaaacaactaactctaccagcaaccagataaaaagtattagaataataacgtgttaaactttaatcaattgtcaatggacacttgcgtgagaaaagagactcaactcaagatccaaaggatgtgttaaggtttctaatactcaataaacgatatgaagtagcaaatcaatagatgagatgaaactatgattagtggtgtttgaaggctcacaagaaccctagcccttatcaaaattgtggttgccaaagagagatcaagaagtccttgatttttgtgagatgttcctttaaattatgaaatcagcgaattttatattcataactcgtagctttgtgtatgcaagattcccttttccaatagttacattcaaacatcgtgggatgctggtatatttccttaaacccttgcattctgctgagaattcaccttttcctgcagctatttaagaaccaccatgaaagtgtactgaattcatgacaagaaaatcgaatttgtcatataaatgtgaaaggtaaaataaataaaacacaaaatgcttgatctctatcacttctttgaaagtccttcatagcctttgaatcataaaagtgatacatgaaacaagacaattatgtgatataaattcatttaaagcaaggtaggaaaattaaattgacatataaaagaaactacttacttgcactccagaatcttttgagaacgactcagaaacattaatagaagttgttgtatatcttcttaacccattagtatatttaaagttatctgattaaatcaagaacacaaaataagactttcaaattaacatttgtttcacctagtatattctgttggcgaggaatgcccataattaacatattctcagtttttcaataaaaaaataaaataaaactggagccaaataatggttgttacgaggcaatgtgaatattcattactgaagtgacacacaattaatagatttcaatcctacaaaaaatagagcaatgtagaattcaagaaatgcagcttatgcttgagagaagagaaaagtagaacctggttcagaatggattacaccaaccatttctccaacatgagcctctagaatcacctgccgagaatcccgattagaacttaaaaagagaaatgcagaacacttaatgggaacacccaccaaatcatggtgaaaatctaaaaactaacatatacctcatcattatacaacccaaacaaaccacgaacctgcatttcgagagtagaaagactttagccacaaaaacataaagaaaagaaaagcagaatgcaatcaaataagaacaaagaggagccttaaacttacctcgaacttcctgctagtgtcaacgtcagtaataaaatctcccttccgaaggacaccctcatatattcttcacgcaattaaaaaaaagaatcaatatttgtatacaccctcatatattacaagtcaggatccgttgacttcttttgtcatacttaacatacttacacctttaatatctctttgtagaaggcgaattaaatcaatccaacctttgaattgaacaaacctataaaagttcatagaattttaaaactatattatattccatcacatcaccttcttttaacgtaaatacgctatttaaaacatatgcaaatatccgttgttaaaacctataccagcgaatgtctaaattgtttgaaatttcgtatgcatgatataagatgattcaacaattggatttcattaattcaccttctacaaagagttattgaagatgtcagtatgctaagttaaacatctggtgtcaacggatcaaaataatttgaaataggatttaaccgcatgtaaaggttgcggtaccttagatatgtcatatgaccactatacttcttcaatgtgaaagccgatgccacaagtgtgtgctggtaaaatagcataacaaacatgtttatcaagattctgtggaacagtcagtcttgctattacgaggattagagagtgaagaagtttgagtgtagagtagcagtctttttttctcaaagaacaatgactgaacattggtcaatggctttgtgacaatgttagcaatctaagactgaacaaaatataagtcatgggtagagatttggcaaaaaaaaattctcaaagaaagaacaaaccaaggtcagattacgagtcaaaacgagactcaaagcgaaagtgatgtgtgcatcgcaataaatgattggtgtggagatgggaaattgcagctcatgaagtaaggataccaccttagaagtagctaaggctttgaatgatatgttaggaccaagacataagaatgccccgaaaggagacctcgtgctgtctatatcagacgtccaatcaacatcacaataggcctacaaacttgggggagacaaagggtaagcatcctgcagaactaaaccatgatccagagcctttcctttgagatatcacaaaattcactttactgcaatagagtaactctacaggggttgagccacaaaccggcagcatttccaacttaggattcctaggaaggaggtatagatgcctttcgtagagaacatatcttctttttggagtaacctctggcatactaagtattcaagatgaccaatctattttagagcaaagacaacattattgtgatttatatttagacatactataagatgttatttatgttttcaacttttaaagtaatttgcctattatcattatcacgattcacttatattggctagaaattttataatgttattgcacatgtattcataaatctaatattgatattaataagtctaaaactgcataggacagattttatgcaattttcaccatcttaatagcgaatctaaatattcacgcatgattagaatataaaaagagatatgcagaacaataacacaatttcgccaaagacaatcgatttcatgggaagatcaacagttatggtgaaaatccaaaaactaaccctttcattatgtcttccaagcaaacgacgaacctgcattttagagacatgaaaatgatcttatgagtgcactaaatgaggtgacaagtaaaagctactggtctttgttgtaccaatatttttttttaaagtacaaaataaggtgacaagacttgaatatgcaagaggctaatattagaagtagggttaattatatacttcaatgtaggagaagtttctttataaaatagtcaatattgctaataggaattgctagtaagaagtagtagcaagtaagaatttgaatccatggactggtaaaaaatggataatctttgttatttatttttccaatgacataatttaattacaacaactacaattgtagattatcataaatgcaatcttgaattgagccatacgtttataattttttttaataaattgagtaataaagacaagattaggtaacagaaaggaaaaaaaaaaaacagatctaaaacacaaaacaccatgtccagaagaccaatgaaatcaaactagaacaaagagaagcctaaaacatattattataagaagtgatttatgttcatgattaaagaaattggggccaatttaaaaactcaaattgaggtttctgacagccaagattaaaaaggataaagtttctttgaatctgataccttttcctccctcttatattggtcgagcgcataattacttgcttcaattggacacggcaaataattaagtacaccatccaaaagaagctgtaacccctgtaccagaaacagcataaaatgaaaaatataagattcttaaaacacaatattaggagttggcctgtaggtataaataattcaagccaaaatcaatcttatcaaaagtggccatggcaaattgcagggaaggatagcagcgccacggtgttttatggcataaatagcagctaaaaatggaggttttttttttccttcttgagcttgattcggaagtgaaagtgtgccacacctatcgcaaagcaaataagtgtgcatatagtcttgcaaatatggaagcaaataaataagtgtgcatatatcattctcctgtcaatattatgcagcttctataagctgatattttgggagtctcgacttaccatattgttttgttgtagttctcctttcttccgggcttcggtcgtgtctaaatctagtggtattcaatcaagattttttatgaaatataaaaagtcttgtagtgtttaaaaacattttaacattggtggacattgtagtgatttttaaatctttagtcgtaaaaaatcttacccgataacatgagatttctcaagattttcctttcttgtctcttagttgtgtgcctttacatctctctcctttctccccatttccccccaacatttgtcttcttttaaaaccaaaattggtatgtcaaagcatcaatgtggaatctatcatccctactgttatcactcaacctaacataaatcgcaaaaatacttggtcattagtgataaatataacacttctttttcatatcaatcactcaaataatgtattcgagagaacgaaaacaacacttcttttggtgcatgtaattcatttagctatttgagattgatattgataggcaaacaactggcaactagaactagggaggttgattcccaacaaggaaaaccttttgcgctaaaagaacgccaaagataaccagcttcaactctagtaattccaatgacagacccaaaaagaacatatgacagagaatgctcttttaatatttataatatgacataatgtttgaccatacccaaaaaattgtagagtgaccttacattcatggaccattgtagccaccatcatactaagaatgctacaagataagtttttgaaaaagaaaacataatgcaactccctcagtctttattcaaggtttggagagggggggaggaatgaagatcaagtccataaaacaaagaattaattcaaggtaattcaacaattaccttatatttgtaagcacttcccatgaatactggtataaacttctccgctatggtggccctacgaactgcttcctacacacacaaaataataacaaaaagaaaactatttacaaaataaggttttggagagggggtgggtgagggagaagtaacaactagttttcatatcagaaaatgcatctgccttcataggtgcatggacaaaaaatggatttcctcaaggatcaaaccctaaaatgtaaaacaagaataaaacatatttgaatttgttctccaacttcatctcaaaacctgaggggtaatactggtcaatattatttattatcatgtaaatttgttagaatgaaaaaatattgacactttccatatacatttgctagaatgacatataatttgatgcggaagaaatattatttaagtatagtaaatattttttgcttagagacctaaagatctttagaaaaaacgtgtccctttttcccatacataaattcaagcatcgtgggatgctagaaaatttccttaaacccttgcaatctgccctgtagaaaatttccttaaacccttgcattcttctctgaatttaccttttcctgcaatcatttaagaatcaacatgaaagtgtacataattcatgataagaaaatcgaatatcacataaatgagaaaggtaaaaggactaaaatacaacctgcttgatctctatgtcacttctttggaagtccttccaagcctttgaatcattaaagcaaggtaggaaaataaaattgatatactataaaagaaactatttacttgcactccaaaatcttttgagaatgactcagaaacattaatagaagttgttgtatatcgtctccacccataagtataattaaatttatctgattaaatcaagaacacaaaataagactttcacattaacatttatttaagctatcatattttgttggagatgaatgcccacaatttcgtatgagattggattgtatagtttaatgggatcaagaagtatagtctcagttttttcaaaaaataaaataaaatggatccgaataaaagttgttacaaggcaatgtgaatattaattactgaagtgacacacaaaaacttcaatgtttagtatattttagccatttttgctcgcattttaggttaatcatatgcagttttcatattcaagccctcaaatgaaatttactctccaataaaaatataatgtccacattttcaaataacttcatcaacgacaaccaaatctaatcccactaagtgggctcggctacatggatcatatgatgtcataatgttatattaaataaagcaatgtagaatgttctattaaataaagcaatgtagaattcaatcttacaaaaaataacttaaaccacccaccaaaaacatattcaagaaaaattcaatcttacaaaaaaaaaaatgtagaattcaagaaatgtggcttatgcttgagggaagagaaaagtagaaactggtcctgaatggattacaccaactatttctccagcatgagcctctagaatcacctgtcgagaatcccgattagaatttaaaaagatatatgcagaaaacttatacaaaagttgtccgaaggacatttaatgggaagatccaccaagtcatggtgaaaatctaaaaactaacatatacctcatcattaatattatacaacccaagcaaacgacgaatctgcaattccagatagaaagacttcagccacaaaatcactagacacaaaatgcaataaatgaaagcagagacatgaaaatgatcttatgagtgcacaaattgaggtgaacaaagaggagccttaaacttacttcaagcttcttgccagtgtcaacatcagtaataagatctcccttccgaaggacaccctcatatattcttcatgcaattaaaaataatcaatacatatatgtagatgtgtatattacgagtcaggattcgttgacgccatgtgccatatttaagatgattcaacaattaattggatttacttaattcaccttctacaaagagttgttgaaaatgtaagtatactaagttcaacaactggtgtcaacggatccagataatttgaaataggatttaaccacatgtaaaggttgtggtaccttagatatgttttacgaccacgcttcttcaatttgaaagccaatgccacaaggtgatgctgttaaaatagcccaacagtattctttgttagtagattaaaaaatatagcataacaaccatatttatcaagattctatggaacagtgcagtctttgctattctgaggtgcataggattaaagagtgaagaagtttgaatgtagaactgagcagtcttttttcaagaccgctgagtttgtctccgagattataaaactgaacattggtcaatgactttgtgagaatgttagcaatctaagaacgagcacgacataagtcataactaaagattcggttctggaatgcaaaatcaaattatgactcacaacaaaagtgatttgattgttgcaataaagtattggtgtggagatgggaacgcgctgctcatgaaataaggttaacaccttaaaagtagctaaggctatgaatcataagttaggaccaaggcataacaatgccctgaaagcagacctcatgttatctacatcagaggtcaaatcaacatcacaataggcctgcaaacatgggggagaaaatagggtaaacatcctgcagaactaaaccaagatccagagtctttccttttaagacatcttgaaattcactatacggcaatataataattctgcaggggttgagctacaaaccggcagcatatacaatttctagtctagttatggtcacatgcaacagtgcaccaactatggatctgcaggcggcaagatcttccatgtagtcatgattcaagaaattgccgccaaattcaaaatataattgcaatttctgacagtcaagataacaaaggataaattttctttgaatctggtaccttttccccattcttagattggtcaagatcataattaccagcaatacttttccctattaccagcaatataaaaaactaaagaggagacacaacgtcacacacaaacaaatgtgcacgaaaggagcaatacttttcatactataaccaacctcattgtgctttcataagcatgctgtgtttggaatttagagataatcgaaataaaagatactttttatacaataatcatcctcattgtgcttccatattttctcaacagtactagagtctagaagtaaaagataaaggcagaagatccttttagaaaggccagagggagagaggattcatcaaattcttcagtgataagtgtgatctcaacaattcaaaatgagccatagaaatgggtgcagttccattcattatcaattcaacctttcctgcaaaccaaggtgactaattacatcaaagaaacaactctagcatccatctttcagctgacacatcacaggtccttccatcccttacaatctcaatttaaatcctttggacaagaagccatccctgtggaagaaaaaaaatgaatctcatccaagtaagttcaaaagtatcacaaaagaacagtggcttaaatatgtattttattcttgcaattaggggtcgtttaaaaattggtctccgtattcgttaatccttgcaaactagtcttgcaatcattaatcatttaaaatttcgtcatttaaccaacttaatcactgccacatcactaattcgatgacgtgacaatcactacccaaagataaaattggaatttcatgtatgacactaatatattcacctctagagatataatcacttcctttaaaaaaaagatatagtcacttattcacaagtttttctagccaactcggggaaaccaatatctctctccattcaacaatcttctttctttcaattgattatggatctaagcttatttttgtatggttttgcaaatttggtgtttattcaataaaatataattttggaacttatccttcgttgtatttttcattacctagtcaatctttataaccattctttcaactagaccatcaacctgtgctccgggtcctaccagtttacacatacgcgtttaaagttacgattattagtctcttcctatattaataagtcaattggttataaccgaatcaatgtaagtaatattttatagcttaaagaatttaaaatcaaaaagaggtcataaatagcttaatagagaactctagttacacactcccacaaaagatcctcctcccatgcaaacatatttatttgtgtaaaacgaaaataaatgctattacacaaagaattctggtttgaatagttgtcataccttcacaatttatttgcctggaatcaaatccaaacatataatcgtttatttgtttgacaattgaattctttgtagttaatatggccttgtcttgaaaataagctatatcagtcatgctatctaatatacttggataggtgatttcgacaatagtcgcaatagaattctttgtgctcattataacttataatattattattgctttccattagtgatatcggaaaaaggagtgcgcgaacttatactacggcttttttgtctcatatgaatacaatgattctctttaggggattcttgtgaaacttgttgcccatttcgtgctcagtttggtttttttccttctatgctttagataacaaagtcaatggttcattttttgcaatgtcgtttagttgtccatcagttaagcgcgcgtgcacacacacacgcacgtgtggagtcctcccaataacaattgcggctcatatcataagtactacacaattatagatgtaaaagcagtgcatgcttgcttgctgatatatacgtgctttgtgtatctcaatacaataacgactattcgttttatgcacacatttattataacaattgcagacctacttgaaactcttattaattattattatgtccatctaatctatctatattaataataagggtgctgctaatcagtgccctcggggcactagataagaaataataaataggcaataaataaatggatttcaattggtttacctaaattaaagtgagatataaatcaactaattaaaatgagatatagttgtatttattgtttgtttttgtagcactaacatcatcaattatgttatttatcatctacaaataaaaacgataatataaaaaaaaaaaaaaaaaacataaagacaagatgtaaaaaaaagaacaatttttttttgaataatacctaataaattgaatgtagcgtaaaaaaaaattgaatattttattttttgaaagataaaaagaaaaaaaagattgaatatggtattgatgatgggagagagatattgaatataacatttattaattttaattagttgaaatattttacactttaattaaggtaaaccaagtgagatccatttatttattgtctatttattgttctttatccggtgcccgggggcaccggttagcactgccctaatagtaatagtaacaacttacatgacatttttttgagatacgttatttgtatcattaaagatggagagcatcaatgtgcattgaatatgaagcgttagatagcttaatctgtcgtgacattatagctatggttagaaagataattgtgagtttaagaccttggttaggttctagaacttatgctattcaatatctacaaaaatctcgaaaatgtaactactacatctattttctacagttgccactaagaccatctccaatggttttcaccattcaacaccacttttttaattcccaacactccacatcattttctctctcttattcaacactcattcaacttttaccctctccaatggtttttcattcaacactctaccccatcattttttattttttattcttatttaatttttgtttttataattacataaaattatcgattataattaaattaaaataatacaattaactatttattttttagtatttttagacaacactgacaaatattaaagcaaacagtcggaactagacaacactgataattattaaagcaaacagtgaggactagacaacactctgataattattaaagcaaacactacggactagacaacactgacaaatattaaagcaaacagtcgggactagacaacactgataatgttgactgaatacaaacaaatatttgattaaaattaattttcaaacagctcacgctccaacttctccaacagctccttcttccggtcatcgagatgctcttcgtcccttaactttagatacattttcatttttttagcttgagtcttttcttgcagcaacttgtttttctgttgtttcaccaaatttaactctttcaattgttcaatctcttgcccttttaattctttgaattgaactcattccttttccaccttctccaatgtctcgcccttgcttttcattttacctttttttttagctgcctccctacccattggacgagcactagatcctacagagtcctcgtgagatctcttagatccactactccctgacccaacatttcctcccatctgactaccataacgtggttgatcacggagagcgtgccattcttcattcaaagtaaattgaatattcttcccacctgcaaataattcctgcgcttttgtcaaaacatcatcttccgaccaaccgcttccttgcatacgcttagcgctttcataagcaccaatccatttatttattattttgctcatataattaaaacggtttcggcaggcaactagatcgcggggagaatcgaatgagcaatactcattacaatactcagcaattttaccccaatatgtttctcctctctgatttctcccgacaacactgcttgttccatatttaatccatgcactaattagaaccaaattttgttgaatgttccatgctggttccttacttctcttgctcttaggagttgaatcttctggagtgacttcatcagcaactgccatgccaccaggagttatttgtgttgaaaattcaggatattggtttgcatcaacactaggaaaattttcattccccattggcatataaccattaaacgggggtgtttgagatggatatctcatcatagatccataatatggatgaaagtttggaacagaggatgactggttgaaatttggtgaaaaaccaaaattaggtacgttttgatgtatgttttgaggacgttggttataaaattgatttggatttggataattgttgggattttcgtagttatatgggtaattagaaaaattttgggtgttgaaatggttattattgggatccatttcacaacaaataagattaaaacttcacttagtttgctaatagaaagataataataagatgaagatagtgaaaaacttggtttttttttctgtgtccaaatcaaatgatccaagtctctatttatagaggggaaaaaatcatgaattttggtaattttttttttttaaattaatttacaacgaaataattgaggtcaaattattaaaatgaaaagaatccagacagccaattaaaatgcgccacgtgtcattatctatccaaaaagtcattctctctccgcacgtctgacgcgtcacgcgccttgtctgcgcgtgtaacgcacgcgcccgttttgggccaattccgcgtcgccacgtgttcggcctggcagggttcaattgtgttgaatcggttcaccttctctctcttctcttccaccattcaacacactttaaacacaccattggaggggaaattggtgttgaatgtctcattaaattggccattggagatggtctaattatcaattacaccattaagcaatatttcatatagatatattaaatagttataaaaataaaaaacatgttgaagtgaaccgcagccgtgcaataaaaagtatagaaaaagtacataaccatcttactaaaataatacataatataagcttggagaaagtcttatacataacaatcaaaatagcagcaaaatcaaattcttggaaattctcacataaggagaagaactcatttgtccaatgctacataaagcacatcattacgacctctggcacctgctccaagctttatgccctgtcctttgtacagcttacgcgaatttcagaacctcacccaagctcctccaaacataatgttgttgtaatactcatcaaaactcaatttcatcgacgattacttcgaattgattgcattttggatccaccaaagttacataatggtacaactcataaccaatgtcttttgtaaggtttgtactcaaaagcatataaccatgcgacacatattgcataacaaaaaaaaatgcaatgtcaattaaacttcactaaatagcgtcaaaaagagcattgtaaacgaagtgcatcaataaacaataaaaatacagaaactaacaaacaacagtatcatatgaccttagtatgtgaatcgatctaaagaaatttattattaaaaaaaaaacacgtcaattaaacttttctaaacaacggcaaaaaagagcaatgtaaaggaagtgcatcaataaacaaaaacaccacgaggttgcaagtagagtttcaaactcacggtaagacatggtagtatgctgctgattttggcaagacaaaaggattgaaggagggtagcaagatttctatgcgtgtggcctcttaagtttaaacattaatatgatgtattaagtggagtactccctccgtatttaaatataagcaaattttgacttttaagttcattcaattaataatgtatgtggtttatattatagactacatacatcattaattgaatgaatctaaaagtcaaaatttgcttatatttaaatacggagggagtagttggcagcacaaagtagactttaatgatgacatgtccacacttggaagggaaattcaaatttgaaaacttgaaatcagcattaaatgatgataccaaagcttgcataacacaccaaggagactttaatgatgacatatccacacttgagagggaaattcaaatttgaaaacttgaaatcagcattaaatgatggtaccaaagcttgcataagacacctaggagatatgtttgggagggaaaacacaaaagcaattagggttaagcatttgcttgacctaattggcttttataataagtatgatatgataggttgtaggaccctgaattcaatcaaacttgccctgtgatgttatttccattgaatttgatatgttcttcaagaggatttatccctcaacatattcatcaaaagttattttagacttatgatctttgaaagagtagtgatataaacattcagcaaatcagtctaggggttaatctttaagagctttttacaaagtaactccgaagtaaattttttgatcaaatggtaattaaaattggtcttcatagtttcaaaaatgattgtttggttgagggggagaaataaatatgtgctacacttttttccttcaccataggtttttccttcatcatgggagaaataaataaccgtgtctaaaatttacattgcaaaggattcacacaagaaaggtacatatacactctttcatgcaatacattgttgatttcaatgttgaatgtcctactattttgccttcactagaattagtttccattgggttttttccctagaaagactttaacaaaatcagttatgattatataatcctaatgcacactcgtttttttttccttgcaaccgatttgcaagtttctacattgagaaactaccggttcattttattgtgataacatcatcacaaattccatgagtaacccaatttggtaatgttcctttgcatcactgactccttatcaaattgttgatcggattttatcagtccttatcactacgtacaaatagttacaatattattcaacacaaacatgaattgaagatactacaatcaaagtaatagaaaaaccagaaaatcaacagcatacacatagcagaaaagaaccaaaatattagtagcaatgttgatatcgattcaacacaccatcaaaaacattgtttaactacttgatcttgactatagtaatttctggaacttcaagcacaatatttggtgcttaccctcacaagcctacgactagcaatataaaaaagtaaaacattgacaattggcaaccaccatagtctatatccaagtagtgaccaatcctatcttcaactttcaaatgacaattgacctcattccatcgactctaacaacagaacacaatatcatataatatcatatgtgtaccaactaaaacattctattgaataccgcattagaacctaatgaagtgttatatggcaaaacacccgcctagcaggccataatccaattcttggtatttgtagcataacgattaaatattatacagcagaaccagaaaatactcaaaacttttcatacagtggcatcaaattaccgaacaacaaagtagctaacgacaaaaaattaattcaattagaaaatcaattcaattcacaacaaaaaagaacccaatttgcacagaaaaatgatgcaggaatgattgattatgcttattgatcctttaaggacaaaaagagacggcgacgacgtggtaatgaacaaagacttacatacacagtgaagaggggacacaacgtcatacaaacaaacgcgcaagaaaggagcaatacttttcatacagtaaccaacctcaatgtgctttcattagcatgttgtaagagtttagaatttaaagataatggaaataaaagatacgttttatacaataaccaacctcattgtgctttcatattttctcaacagtactagagtctagaagtaaaagacaaaagcagaagatccatttagaaaggccagagggagagaggattcatcaaattcttcagagtgataaatgtgatattagacaaaattcaaaatgagccataaaaatgggtgcagcttcattctttatcaattcaacctttcccacaaaccaaggtgactaattacatcacaagaagcaacttgagcatcatgtttcagttggcacatcacaggtccttccatcccttacaatctcaatttaaatcctttggacaagaaaccatccctgtggaaagaaagaaagaaaaaaaaaatgaatctcatcgaagtaagttcaacaatttcacaaaagaatagttgataaaaagccatacttttgaaaggaattatgcatatttggagtaatttcatggtaggccaatttctaaacaactttcttctctttacataccacggcagggaagaataggctcaacattcagaaataagcttgtcaagataattatttaggagcgaagatctcaaagaatgaatgaaaaaaagcaattcataccaaaattctttctttaatctaattttaaagaaaggaatcctgcagattcttttttatgcttccaattctctaaacaaggcaacaacttgtttaaattgaccaacgatgcaatagccatagattagattatgatatatgatacaactacgcatgatcttcttaataatttcagaatataaatcttttgattcgtttgattcgagttgtcgagctaaactaagttgttcgtgagctttaattgaactaagtcgttcgtgaaaaaactagggacgaactcgatttgagttgagttgaaccaggacgaaccaagttcggctcagatcaactcattcaaggtctactttggttagatcattcaattaacccttttatacttgttgatctatcagcaaggtgaactgatggcaaaagggacaaggattttccgttgtcaaggaaatcttgcagttatgaaatagtcatatttccactatccaatgaagattggatagctacgatttaaactccaactaatcagctttaaagtgtaactttcatcggatgactgagatttgcaattgcatcaattagaaactctaggaagtccatttccaaactgtaccacttcattctcattttttttatgagcttctgcattttagttctgctgctgataacaaatggtgtatcatgatagaacccacaaattcatgagaatgaaattgtattttatttaatgaaaaatgatgagcttaagagctctacaatggtgaaaacagaaactgaaaggactaaacgtctaaaccagagtcaaagactactaatccagagcatagctcctaccagagagttgaatctcctaacaactatttgaaagaaaggtactgaatgccttctacactctctcattctctcttataaagagacgtgatcgccttaaactgtggctctaatcactaattaattattatcaattattctcctaaaagtgtgggtcaaaccactaattaaccattactactaagattgtggatcaagccaccaagctcccctcagcttctataaccttctagaaggtgtaggagtgtaacgtgtgtgatttggacccaatcattgggtgaccctagtgtctataacatatcaactctctttgcaaagtaaggtggtttttttttgggggtttgtatagtaaggtgctaatatatataaatctaaatcagactttctcaaggaagtagctgatgcatatatggccagcatcacattcatttacgttccaattgttgtgtggccctaatcattttagggtcatattcaatttaaagaaatactacttttcctatactggatctccaacaggttgttgaaaaagccctacacatcaaccatgagaaagtactgcaaccccgtagaaagggaggaatcaatacatcatcgtccccgtagaaagggagaaatgcatataatatcatcaatgaataatttaaaaaaaaaaaaacaaaaaagaatgatataaaaacatagacaaacttataagatatggtgtctgttaaggcatcagtgtcaatatatagttgtaaattatatcataaaccaatgtagcagataatcagccctgtatttttaaatcatggctgcagttgaagagtatatctactggttctgatgccccaaagaaaatgtcacaagtcaagactatcacatgcataatcaaagagcaaaaaacaagaaacataaatagctgtctgttcaaagggaataaataaaccaaatacaaacatgatcaaagaatgcattaaacattataagcaaaagcatagtccttcaattagcaaaccaaaaagcatatttactacactcaacaaacaagttcctttgcactattgttattttaagagagagagagagagagagagagagagagagagagagagagagagagagagagactaattgttctgcaaccattgtctttatgcttcgtggaacaagcccacattaaatagtctgttcattataaccataaccatattaatcggaatagaattgtgctggcctaggtttgtaacgttgcaatgtttccaccatttgtgcaatcttatcaacttgtccttctactttgaataggtgatcaatcaccgagtgataaacagcaacaataggttcgacaccaccgtttacagattgcacttttagcagctttcgaccctccttcagttactaaagtaggaacattaacacttgaatccatgcattttaatgcctctaattctctaaacaaggcaattgctggtttaaattgaccgacgatacaataaccatagattagattatgatatgtgaatgtgatacaactacgcgtgatcttcttaaaaatcatttgtgaatataaatcataggcttggtttacaggtctacctttacatagtctgaatatgattatgctatagaatgacctgacatcgtttaactatcaaggtcaactcttaaactgacacgtaacatgcattgtcttttttagtttcttttggaatcaagcatacacacattgtcaaatacatgtacataaacaaaggtagaaacttcaaagtaattagagaatctttatcctatgataagacttcaatttgaccttgagcaaagtttagctacatatatcatatgatgggtcattgggtccattgaactgatattaacttgtacaaagcaagaactcaaaacttagaaaataaataagaagtaccttctcttggttgttcgattctccttcctacaattatcaacagggaaggaagttggtacaagactttcaacacaaatcacagtgtcattccaattaggaactccttcaacataaataacttgttcactcttcaaatcataccaaaagagcttctgatgatctacctccaacaaaacctcaattgcttccaacagaaccttacttccatcaccggaataacctagaggcctcgaagctgtcaaatgtgaatggaaacaagatttgaccaaagtaaaaagtttacaccaactatctctagatccatattctttcatcacccaaccatcaatattagtagtttcataattcaaaatcatacaaagacaccctcccaaaacagcaacagtcatttgaaaactctcattactattaacctcttcacctcttatttcagcaggaaggggaacctcaattgaaaatttcaagtgtgaggttaaatgcaacaattaaagaaggatgtaaccgatctagtttcttcgtcattatcaagtgaatagaattctcaacaaaaacccccatgctgttaacatagtaaagagcgtagggcatagtaggaattattttcactctatataagagtcttcttatatgctatttctctatataaagaaaatatatatggtttttggatggattttcactttcaattatacaccattaaacacatatacttataacaataaaaataatagtggacaaacggatatggagtgtttgtccgaacattagtgtggataataataatttaagtgagttatgcaaaagacacattgacgtcaaagtaagattggctgctaatagatagcaaagtgagtaattagtaagtataagtatgttaatatgaggatgtactcagtgtatcgaaacccgcaattgaaagggttttttaaaagaaaaattactatcccatacagaaaaaatttggaaaggaatttttacgtcgtggttagttggtcctgacgtgaaaactcgtttgtaaacatcatcccacggccgcctagacttgtagcttgggggatgagatgaaattctaaaggatttttgatcatacaactcgaaggccaaatcactttgaggatcaaatctatctcaaaccgactaagtatacccctatttgtgatcacattttccatcgttgcctaaactcagtttttattgtagtttctgtctaaaagtaagtaaaatgatagaattgttgattattataaaattttgataaatgattttgcaacttaaaaacgataagtttactatcaaacaattgatatttatcctcgtgagcttaactcagttggtagggacaatgcgtaccttatgcaaggttcagggttcaaaccccgaccaccaaccaaaaaaaaaaacaattgacatcccttcaaaatgttagccttatgccccaaaattcctctaaacagccctaccccttctttacttattacttataacatggcaaagcacaactttaaaaccgcacttgcgttgcagtgtttgaaaggtttgttttcctctctgttgcatccattctttaaatcaacaatgtattacatcgcacaaatcgatgtatatatggtattgacttgaactttgtgtacgtttatacgtgtgaatccttgcactcattcaagcattaatcatggcatttaaccattctcttcattcatccattcactctctggacctcattctcttattccacttcttccatattgccttgaagcatatatttgtgcattagtatattcgtctctagagatatagtcacttcccttaaaaaaaagacagtcacttattcacaagtttttctagccaactcggggaaaccgatattcctctccatttcagaatcttctttctttcatttgattatggatctaagctgattcttcgttgtctttatcattacctagtcaatcattataaccattctttcaataggttgtagaactacaatcaaacttgccatgtgatgttatttccactgaattttatatgttcttcaataccgatttgtacctgaacatatctatgggaagacattttgatcaaatggttaaaaaaagtggtcttcatcgcacaattaaggaactaccggttctttttattgtgatagcagcatcaacacaaattccatgagtaaaatgttcctttgcatcacatatatgcacaaaccatttggtaatgttcctttgcatcacatatatgcacaaaccactagcacctgcttcatatacaaactacaacacgcccagtctcagatgacattaaaaatgataagttatttgccactgactaacatccctccaactccccacaatgcactagccacctagaactattattatagagtacaacactgactctatcaaattgttgataagattttatctagccttatcactacatagtacagacaaataattacaatagtattcagcacaaacatgaattgaagacactgcaatcaagtagcagaaaaactaaaaaatcagtagcatacacatagcaggaagaaaaaaataatagtagcaaatttcttgttgctatcgattaaacacatcaccaaaagcattgtctaactacttgatttgtcaatagtaatttctggaacttcaaacacaatatttagtgcttaacctcccttatattcaacacatcaccaaaagcattgtataactactttgattttatctatagtaatttctggaacttcaagcacaatatatagtgcttaaccagcaatagaaaaaaagaaaaacattgacaatcactgtagtctatatccaagtactagggcataacctcccttatatctcttcctaagttgaatatgatgcagcctaccaccagcaatataaaaaagtaaaacattgacaattgacaaccaccacggtctatatccaactacagcgaccaatcctattcatttagaaatggcaatcgacagtgtcgactataacaacagtacacattatcatacgcatgccaattaaatcatcattttactgaatactgcatgagaaccaaatgaactgttatacagtaaaacattcacctagcaggccacaatccaattcttacatacagcagaagaaatcaatttcatacaacagcattgcagtaaagatgatatttttttttttttttgacaaggtaaagatgatatttatagcataacgattaaacttcatacaacataacgaaaaaaaaaaaactaaatacgtttcatagagtagcatcaaagtagccaacaaaacaaatcaattcaacttgcatagaaaatcaattcaatttgcaacaaaaaaaaagtacgcaatttgcacagcaaaacaattaaggaatgataatactttaaggacaaacagagacttacacacacaatgaagagagacacaacgtcacaaacaaacgcgcacaaaaggagcaatacttttcatataataaccaacctcattgtgctttcatacgcatgttaatggaaataaaagacactttttatacaataaccaacctcattgtgctttccatatattcgcaacagtactagaagtaaaatataaaagcaaatgatccttttagataggccagagggagagaggattcatcatcaaattcttcacaccttaatgtgagatttttacacaaggataagtgtgatctcagacttacaactcaaaatgagagataaaaatgggtgcagccccattcattatcaattcaacctttgccacagaccttcattaccaaaaacaacttgagcatcttgtttcagctgcacatcataggtccttccttccatccatcccttacaatttcagtttaaatcctcgggacaagaagtcgtccctgtgagaaaaaaaatgaatctcatcaaagtaagttcaacaatattatatcacaaaagaatagttgataaaaacccatgcttttgaatgaaactatgcataaatttggagtaatttcatggtaggctaatttccaaataacaatttactacactttacataccacagcagggtggaatagtctccacattcagaaataagttggccaaaataattatttaggagtgaaaatctcaaacaatgaatgaaacaaaagcaattcataccaaaattcgttctttaagtaaattctttatctagtcacaaggtcaactcctaaactaaaacatagcatacaatgttattaattttttgtttcttttgaaatccagcatacacacattgtcacatatacataaacaaaggtagattcaaagtaattagagaatcaaatccttgtcctataacaagaattcaatttaacctttagcatagagtttaactacatatattatatgatggccattgaactgacatcaacttatacatagcaaaaactcaaaaaaaaactcatataatcaataagtaccttctcttggtcttgcttgtacgattctccttcttacaagtatcaacagggaagaaaggtgatacgagacttccaacataaaacatagcttcatcaaaattaggaattccttcaacataactaacttgttcactcttgagatcataccaaaacagcttcctaagagttttatccaagttaggatttgcttccaacagaaccttacctccatcactagaataacataacggcctcaaaaagtccaaaggtgaattgaaacacgatttaaccaaagtaaaaagtttacaccaactatctctagatccatagtctttcatcacccaaacatcaattttagtagcttcataattcacagacatgcaaagacacccacccaaaacagcaacacgaattttaaaactcccattactattactgttaacctcttcaccttctaattcaacgggaagaggaacctcattgaaaatttcaagtgtgaggttaaatgcgacaattaaccaaggatatgacccatcaagtttcttggccataacccagtgaagagaattctgaacaaaaacccccatggcttgaacatattgaagagcgtacggcatactgggaattgttttccacgagtttgttttcaagctaaagagactgacatgtgaatcataaaaaatgttttgtcgagcaaataaccaagtgattctgaggagtttgtaatcggcggtgaatggatcaaaaccgaagccgtgaacgcatataccgccgcgattggggttgtcagagtcctcaatgttagggataggaagagggataaaagggatggtccgatctttgcgggtgttagggttccagattgtggtttcgagaacgccatgagggtgcttgaaaacaatttctccgtcggaaagggcgaggaggccgttacatgaaccaataagaggaaaggtgcggttgccttttagagagaaaagggaaagcataatgttagtgcttaaaggatggttgagcggtatcattgatttagagaaatcatcatcatcgacttggagttggtagagatcggatttgtgtcgaaggattataaatcgattgaggggattatttttgaggtaaaggttagtgaagttgtgagaatcgattaaggatttaagtgattttgaagttgatcggaatcggagaagtgattgaaccggtaagcgggagaagatttcggcaagtatttccggcggaagaacatcagccgccattggaaggaaggagggtttagagtttttcctttctttgatttgtacaaccaaatgcagctttggtaggaaggctctagagtctagggtttacgagtttttcatttcgtttctccctcccttttttgtaagcatttatttatctttttatatattccttggttccaaattataaattttcagagtaatcattccttatttattaattaattttgttaaatctgcatcaacttaacctaatttttttccttcaattaatcctttattaggtttacaacaactcaatcctaatattttgactctaaatttacatcatcccttactccttaatcttatcccttactccttaatcttattaggttgcaacaacttaaccctaaaaatttgactctaaaattgcagcattaaatgctaagtgagataaccctacaaaaatttggctctaaagtgtttgggaatttgaatttcaaattattttattgaaacaaacctactagcatttaccaatatatgttattaataacaatgatattttccaaagcaaaatttgacccagatatgctctttaaaattcaatttctccctcattcactacttattttatgtatttcgaaattagcaattagtctagctattgtcgtgtctcttcctattttcatgcaacatttagtgcattctttactgaataattggacggttccatgatatcagatgttagaatgcattaaaattcctcagatttcaatattacggctcccttcttccctcatcactcaccactcaccactctgtagtctctctctttctactgatcttctttcatttgcttctacaataatttctcagcaacgagcaactcaccattttctcatcgaaaacctgtccccccaccttttcatctgtgtcgctgaactcagtggcgggttgaggtaatgctaaatatgtttctctaatggtagtagatcatttgctcttaaattggagaaattgaaccgtttacttttctttgatttttattgttcttactaatttgtattttcttttcttttcttttctttttttcgattttgatttgaactgaagtacctgtaacttttgatttaaacggaagtcacaatatatcatggactttgtggttttgaaattaattcacggttttggatattcaattttgatttgatttcaactttgcttccaaccaggtactttcgtttttctttctaatgctgtaaatattccacaaattattctacaatattgctgcttatagttttcttttctttttgatacttttattctgttttgcttgcaattatgtggttcctttttatctttacctcccttattttagttctttttagttgtgaccgagacatttagtcaaattgtggaatttgatggatagattgttagaaatattgtttgttattcaaaataagcaccaatctattctgtgactatggtttttgtgtgcttaattcccgtctcttcaaaagtcacaacaggagatttagttgttgatttgtttatgttgattatgttttagtttgagatattgttattgttactatgttaaaatagatggtactggtttatcttatgtcaattcgtaattatttttcgagtaatgatatataaataccctttattcccatacactcttgtacaccccatgcattaggaagagagaagaggagagaaaagagaaagtgtgcttgtgcggggtccacatgactacgtgtcagatttttgtgtgggtgtcaaagggtgtattgccacctaagggtgtctatgtatcattactcttatttttctcaccgttgatgacttgatgtcgtgttggccgtgtcagatattagatactttcaaaaatgtagatgctacatagaatttaacactttttatgttgtaggatttgtttcgtagttgcagagattttgttattggaagggattagtgtagaatcgggccatttaaaaaattggtctaatcatagaacaacctgatacaccgtcagagaaggtttgttctcagctgctggggtattttatttggtcttttctgtcaatcaggtgcttcttccattggttctatccattcaacctttttatacatattcgttgctagatacacaattcattaaacctttgtgttcacttattcaattgtttaatgatgttttgacagtggggctgctggaatcctagggaagaatttgaagaatctttctgatattttgaaagagtgaagaatgcttataatgggattttattttgttgcttttttatatgtatataatgttgatgttgattttttttatctgttatcactaatgaatttctctttgtccctgcagaatcaaaatgaaataaacaatggcctgcataacttcttcactatagtttggagcttttcaagtaattccctgtaactttaggtttgtgatcaattatttgtgaaactgacatctaaattaagcatcaattatttgtgagtagtatatgtgttaattcctttttgtgaaatatgttaatgaatatttcagcttacataatgtgttttaacactttggaaattccaaaatgttccaaaatttttttagctcattgtagtgattttatcatgtcttctttttgttttttccctgctatttacaccaaaaaagaaaaacgaaagaaataaaaatgactgggtcacatgatcctaaatgcgtctcacatgatttaccataatgatataatttaattttttggaacctttgaaagacaatattaagattaatttctctagtaaacttatattcaattaaaatataatttgttttgtataaaattatctcaatttcttggataataaatatgaccaatatatttaattttagtgaggcacattattagaaacaaagagaccaaaaaaatatcataaatttattcgataataatattttggcacaatattaagaaaaaccaaaaaatttatagggagggtgtatatagaaatctttgtataaaagggtgtatatatagcaaattgagacaaaagggccataacattaacagacttcagcacagcctttcaacatgtttgggcctttcagtaaacattactatacacacttccaaaaactaaatatttctatatacacccccaatcgtaattttttttagggcccctaccggcaagggcagtaggccgtcgcacccccggcctatgcctaggtccggccctggcagtgtgtacacggtatgttacaaagaacaaaacatcatcactgaaaaaacaacgtttaatttgacacttttttaaacaatgctaaaacacaacatttgacttaaaacttggtgcaatggagtgaggaaggagcacaatacaactcccataaaccaaagtatctcgtcttttggaatttgcggttagcactaggaaacatgttataagataaatctttcgctctctgaatagttattgtcgtgtttgtgggttgtcgactattgtaaattcttaaatcaaacatgttgtcttaaacactttgtctttgcaaacctcatgcatggggagctatagaccgtcatacttagggcaattactcggttactacttccaaaagcatattaaacccatcaccaaataagacaatccataatctgaatatacgattgcccataagaacgaggcacacgggtgtttactcggtcaatagagcactatcacaatgttagaaaaacaaggcaacgattatccgagacaagtctacttgaccgggacacaacgagtataaacgtcgacacaccacctctctctctctacacacacccctcattcaagaatcccaactcactttgatcatgaaacatcatcgacaaggcctcatatgtaaccattgtcaataactcataatcgtcgatccatcgtcacataatgtaaccattagacacaaatggatcaggccgatattattggaacaagaccttaaaccgtgcttcacaccaaagttttaaatttgattttttcggatgataatttagatgagctacttttataggagtaataatgcagggtttgatttttagattacttccaagcaggggatccaaactgctgcttccattccacccaaaaagcaatcggcaatgggatgtctcttttttctgacacataagagggacacagtaaagataatttaaatcggtataaataaagaggattcaaagacagacccagaagagacgacaaggacttatcataaacgtgggtcctaccctaagaccccctctccaatatcaatcaagttttttacacaaagtacacaacaaacactgatacatcatatcaccatatccctcatcacaccctatcatttccctagcccaaaatctacgtcaaaaatcaacaactccaataacattaaaaaaaaaacaaaaaaaaaaacatcattaacccttcaaacctcaaaaatcaaacacactctctctctgttcggtgttgttcagaaaaaaagtatcaaacactttctctctctatctttttcatctttttgttcaaccatgaagaagctctaccataaagcgacggttcatccatcaccaccggtgataaccgaccaactttccttccttccggcaaccatccttaccctagcggcggcgctatcacctgaagacagagaagtcttagcttatctcatctattgttcatccacaacagccccaccaactttcagcaatttctccggaaacccacgacggagcacggtcaagacaggcgatcatgcaactctgttcaactgcagctgtttcagatgttatacaagttactgggtcagatgggacgagtcaccgaatcgtcaactcatacatgagatcatcgatgcttttgaggagtggttggttcagagtggtaaaggtggaaagaaagggaaaggaaagaaagagaagaggaacaagaaagggtttgaaaataagaataagaataagaattctggtgagttgaatcggtctgtgttgaacgagtcaactactgagttagaatctgttggtgaaagtagtaatagtagtaacaatagtggtggaagtgaaaatagtgaaaaggttgttgttgttgttgctgatgaagaaaaagggtcagtgagaaaatttgtgagtttcattggagaaaggatttggggtgtttggggatagtgaaatgaatttggaagagaaaaataatgaagaaaatttcatataatgaagaacaagaagaagaagaaattaaggtaggtttgtttagtctacccttttttattttctttttttcttttaattttttgttaaaatctttttgtacattaatcatcaccatgttatgtttaaaaagaaaaaaatgtttatttggttttgtgatgaatgagttagattttgttgttaaacatcagacaaatgttctggtttatgttttttgcttcaatggatatatctcattctaattctaattctaattccaaggcattgtaaattattttgaactttttatttactttttctagaaattatggaaaaattgactcttaattttgggtacagaactcattttcaattgtagaaaactgggaaaaaaacattttatttccttattgtaaatattggttgaatccttgttcattgtaagtattcaatatgcagtaataatgttcattgttttaggtcaaaaattgtcaattccttaattcattgcttaaactagatgtttgaatttgactacttcatacacttgccaccaggtagattttgtggtcttttttattttttataaatagttttagtggtcttcatttatctctttcggtcgaggatttatttaataataaatatacaagtaaattgcacaagaatgtgatgggcaaaatttaaaatatgtattgctcgcatatattgacttttttaaatgtgcgttgaattaaattaaattaaattaatttgaaagtaaataaattttaaatttgaacagtaccgcctaagcattctgcatatcagctcttaattttcgtttggcttaaatgcagctttaattctccatttttcctaaaacatgattttagtcttttatttcaaaattgtttttgtctgcatttaacttactgttacaacgttggttctggtgaatttcggcttttaaaattggtgaagtagtgtcaataatttcacaaattcatcacttttggggtcaaaactcgcattttttggggccaaaagtgtgaatgggaccataattgaaacactgaaaatggtttaccaaaatccatattttaaaatgaagggctaaaatcgtaaaatagaggacggaaaatgtatccaaggaccaagggagtatttttaaaatgaaggactaaaacgtccatatggatattctgtgcccgttcttttgctttatttatcgcgctaatgcttagaaataacaaacaataacatttattttcttttttgaagaaagcaactcaatccatttctccgtcctaaattgtaagacgttttggccatttcacatgtattaagaaagttaattaatattgcgtggggaagaaaaattatgagttgttttacagaattatccctaataaatggtatggaaaagataaataaattaattgaaagaagagagagtaattaatagttaaggataaaatagaaaaaataacattaatatttcattgatattgtaaagcgacatataatttaagataatattttttttcaaagcgacatacaatttgggacggagagagtattagctaaaataaactaaatacatgatgttatatcttcataagcgtcagaaccaacagtacatgggtttatctaacctaattcttgaacgactgcatcaacttatcttcattaaactcaacatttgagtttttttaacatatttttaaacaactgtatacaaagagtttactatgctacttaacatttgagttttttttaacatatttttaaacaactgtatacaaagagtttactatgatactgtttgataagacagttttattagtttataagttcgtttgacaaaaaaacattctatttggtaacacttattcaccatgaactaatagtttatttcacgagcttataaactattcttcagaagctatttcaagtagcgtttgagcttatagcttacagcttctcacttttaatttcagttttactcttattattctaactaaattccatttttttacccttaatcatttattatattataaagtaaaatacacgttttaaatatgaatgctattttttcatgagggaaagatctttttttctcacaatagaaaagatattttttttactgcacgtttaagttttttttcgttacaattgttacgtttttttttttgtgtgactatttcaatatttaatgtcaccttttttttttgcgaaatgacacgttatttttgtgtgatatatcaaaattgtagagaataagaacaaggataataattttctccaacaaaaaaaaaaataagaacgttgcgtttaccttcactatatatatttttgtacaaactatattttatttattgttacgtttaaacttttttagactttatatctaatttttttagtcaaatgaaaataaatataaattaacattttggaattaaaaattaatttaataataatattaaattaacaatttttaatttttaaatactttaaacattaatttatataaactttattatgaattaaaaatgtccttttatgtaattttacatccatcagctaattgaactgctaattttatcaaacacttcaagtagcttatcacatatcattcatcagctataagccatcagctataagctatcagctatcagccatcagctataagttatcagctagcttatcagtcatccgctatttttaccatacagagcctatatagccaaattcactattattatctacatatggtaacaaattaacaatagtaatataacaaatattatataaatttttttttttttacaaatatataatataaattttatctttttgaatgacatccacaatataatatttttacagacaaatttattcgatggtataattgaaagcgaaaaaaatcatcacaaagtgacaaaactatgtattttctttatagatagatagataaaatattagagttgtattatttgaacaattattcctgtgataacttatgaaaaaaaatcataaatttataaagaaaaatagatatttatatgaaagtcaaaacaataataaaaaaaaaggtagaaacataatgcaaatatgaaagaaaaaattgtcacaaaagttgtcaaaaataattgtacaaatatcatttttcaaattattaataaactttgcatatatccgaactcttttgataaatttggatgatcacctttattagatctcatctcctataatttataaataaacctaggtgatccacaagtcctacatcaactgataaaatgttaaaattgttaagctgaatgttataaccggagtccgaacctcgatacctccacttgtgtgtggagtttaggatgactttgccatttcgtttatctaaaaaaaaaaaaaaaaaacctaagtgaataattaaagatttacttgacactgaatatttaaaaagttctgcttcatatttgaatatattatgtaataatttctcattaataaaaaatattgagattgttattcaacgcaaaatacttgctcaatttttttataaagttcacctttaacatttttttcaaacaaaaaaaagcataaaatcaaagaatctgcaaattcgaactccaattcaatatatattatctgatgtttctataaactgaattatgtttatctgagactatttgctcgttttctattataaattctttgacattttccacacatgttaaaataattttaattttttgacaaaaaatataggctaaacttcacttttcgccctctaagttttaaaatgttgcgattttgaccccctattaaaaaaaatggcaaaagtgactccctatgtttagggaaatatgctcttttgacctcctaacataagggaaatatgctttttgacctcttatctttacaaaaagttgcgattatggcccttttttgggacacggggcgtcttctcagcaattttgagatgaagggggccaaaattgccattttttttaataggggatcaaaatcgcaacattttaaaacataggaggcgaaaaatgcactttagccaaaaatatatatataatttgacgtgtatatgaacaaaatcttttgagtcatatacgacgaagttatccttgtacttttgactggactaaatgaggattgaccggataggagatcatattcaaattcataactaacaattgtcatgcaagcagtaaggtttcaaattttattctgaaatgaggtgaaaattgtatggcttctactacatggaaaaaaacattgtagtccaaaaagactacacgcaataaatgaaggtatttatgactcacataattagaaattgtttaggatagagagcacatttgctcaaaaaaaaggatagagagcacacctcattttgtaaaaaaaaaaaaacagagagagagagcacacctcattcataataatggctgaaatggttgtttcaaattatataaatagtaacatctttggtaaaaaaaaactataaataacttaagttaaaacagagtagaaaaatataatgaccttgatgtagatacaaaggaataaagcgttggataaaatgatttttctaggttgctccttgttgaattggggtaatttcttgtaaacgtaggtcagtggaaagagatagtgcataatatatacaaagttcggtgtttgaaccgaacaaaaaaaaaaaaaagaatttcttcgttttttctatagtcagttgccattttcatcttaaaaatatcaagcattttcatctctttcatttaacatttgatttacatcttctttgtttcataatcgaatcctactagtcattagaggtaaattaccctcaattcatcaagagataatgttggtctcgtcaatataattttttcttacgatcagttaccattttcatatgaagaacaacaaacttcttcatctccatcattcaacatatgatctccatctcattcgttacaaaatcgaatcatgcttgtcattaagggtaaattactctcaatttatcaagaggtagcgttggcatcatcaacttgagatcagagaataaccaaatcgtttgcgataccttagggatcgatcaactattgatcttggaacttatcttggggccctgatgatccatcaatgcattaccaagaaacttagagataaaattacttagaataccttttcttttctgatagataaaacgagtaagaaactatcttgttggtagcgaattcgctctcttttttgctcgtcgagaaatcttaaggcaatctttcttttttgaggaaaggaaatattccctctcatgcatccatttgtgaggagattgaacagatttgcaggaattttatttggggttccactactaattagagaaaaatataatttaatagcttcgcacaaaattgtttacctaaagaagagggagggttcggttttaggaatatgtgattttttttttttttttttgaagaaggaatatgtgaattcttaaccaagcatatatatgcacaaattggctctgcaactagtttctaatcctggaagctttgaatttgagtgatgagagctaatgataatattggtaatatcccttagaagttgttgttcattcgctttggaatgatataaattctttaaaaatctcacaacattcaaatcgtaagcttgattggtgacattgcacatctaactgagttatattaccacaatattcatcaccctaatgagtttatagatcgagtcaccagatgaagttaaattcactaggaacccctcttgaaggtgcttatgaagttaatgttgataaatgatatgattgtttaagcatgttaaattaagtaattattcttttaaaatgttaaatttttcaattttcaatgcattaattacacaaatttttataaaaagttattatttaaggttcttaatccgagcattagttaacattttccatcttttaatttagttaggattatttgtcatttctttttttaaaaaatgctaaccgttgctctgaggacactagttaaaaaatctaaaatggaagttctgtcttaaagacattgaaggtttaaaaagtaatcttttctacataaaatttattaattatttttatttttaaatctttaatcattgctatgagggctggttaacaagacccttgaaaaaacacaagatgacaaagagataatagtagggtaggtgttgcaaagaacaaggtcggcaaaagtaagggcttcgatttaagcgaaggaggcagaacaaataaactccactattcaatttgacacttgactccaaaatctctttttccttggacttcgaggtgtacccatttttatttattctttctatgatactctttcaaatttcaacatcacttccaccgcttttactacatttttcccttttataatcacttactatgaattttagcataatttctttccattattttgctttcggcttttgttcggttcttgtgaaaggattagtcgtttttataaaactttatgcataacgcacacccaagtcacccatccaataatcacaagattcacaacattttttattttttgatcaaatcacaacaattttatatgagaaatgatattttgacaaccattttgtgataatctatgtaacaactttatctctcatattcacattatttttttactttcattctctattgttttggtttccgtgtcactatctacttttctttataaattagtggttatcacaaaaattatcattcaattaattgttcaaataacacacctcaatatcattttatatatactgtgatctttttgacatgcatgatattgatattcatcattcatgtatgtatgttctctctagaggcggcaaacgggagaattctcttagagactatataagttgtaacttgtaattcacactcattcgttaattaatgagaatattattcatgtcttttgattgcagttctactacttttgatgattttttggttactcctctattaattcttatgtatgactccttttctctactgattctattgtttggatggttctctttttgattggttgatggtgatggtttatgctgctctctacgaagataactcactttggttggttgctgcttttcattggtttttaacatgtgcaaatttgcacatgatggaaaaactaaaacatactatctccggtcatatttataagagaaatttaggtcaaaaaaagtgaatgtatttggactgaatttttaactagatacatcaacttttgttgacccaaaattctcttataaataggaccggagggagtattaaagaaatggtcttgttaacaagtgccccgaaagctttgtttaaaggccacaaaaagaaattatgttttgaaaatacaagtcaaacacttgttaaagtcataaccgtacaattttcaatgtaaaagttgttacttttatatgattaaacaatgctcttaggacacttgttaacattttccttaaacaaattggcctcgggttcaacgcgttttgtcattattgagtagtatttactctctccgtcccaaattgtatgtcaatttagaaaaaatatttgtcccaaattgtatgtcactttagaataccaatgaaacattaatgttacttttcctattatatctttaactatttattactctttcttttttcaattctttcatttatctttctcatatcatttattaaggataattttgtaaaacaactcataatatctctttcccacgcaatattaattacatttcttaatatgtgtgaaatgcccaaaacattatacaatttgggatggagggagcagtaaataatgtatggctatgaaaatttaaaatttcattgtgcttgtcaataagtaaaatatcaatatgactgaaaattaattagtcaatgagttgaccagtgtttatcaacgtttttctccgtcaacttatgttaaggaagaaacgatctccgtcaaccatgtgttttcaaaatattctgaaacacgttatatgaagtaaatgaaaaacaaacctaagaaaataaaacataacaaaaaggcataaaaaacttacagctgaaggtaaattacaataaaattgagttgtatttattaacgtgtctaaaatgagtggtataaaatttcgcatgaaaaagtatttaataatagctcaccttttttttttggagaggaataaataaatagttcactctagaccaaaaaataaaataaataaataaataaatagttcacttattcttgcaaaaaacgtgctaaaaatttctcatgacaaagtattaaataataatagttcacatgttcctgcaaaagaaaatagtttacttgttgagcacgtaagaaaaatacggtctacatcttatcctaaaaaagatttatgaattgcacaaaagaagttggaatacaatatcagcctcacgtcagttaaattgatcggctgaatgaatcaaacctcaaaccaaaatctttttggtacaatcatttagtcacaagttgagagaactaattttttcttgatgctatcctcgttttaaaatgctgtctgtgtgtgtacataatcgttgtgtgatgcatattaaaaaaaaagtgtgatgcataccttcgtggttcttgctttgtagtaaaggttaaattgacaatatgaactcattgtgggactaagacagaagtcaagattttgcagcgtgtttcagtggttgttgagaggggaaaaaactctatgttgtctctctttgactgactgagtatgtagtctctatttttcccattaattttttttttcttcttttaaacaaaatttcctatcatgtttatatgttatgtccagggccgtcccaaagaattcgttggctcagctctgggaattaaaagaggccagtgataaaatcaaaacgtattgtttttaaaagaacaatgttctatttatatttttttaaaagataaatataaggtgccgtatatatgcggtacacccaaaggtgaaaatgactaccgtatacatgaggaacccccacctaacacaaaaagcaagaggacctaattctaataagcctaaaagactaaaacaaggggacataaaatagctgggggtattaaaaaaaattggagggcagtaatactaatgggtattaaaaaaaaattgaggcccatgactgagggaggcccagctctgttgagctgtttgcaccccctcaggaccgggcctggttatgtccatagaaaaatatgatacctcatacaaatggagtttggtttagttggtaagagttgatttatattttgtcatgtaaatgttcttcgttccactaatttttaagtaaacattaaattttgtcatattataagaaaatgtcacaacttttaagtataattattgtttaaaagaattttctatccctcttttagtggttagttggtgtattttatgttttataatttgttactatggtatatttgtaaaaatattaaaaaaactacattaattaatgattgtattaattttaatattttttgattttgttttttataataaggaccgatgagtaatatttatatttattaaaaagatgttgatatttatataattttctatatacgattagtcacagtcgaaaatcagatagatggacacgttaatgtttgtcattttgctaatagtagcagcatgagtagtagtaatctatgctatatataaagaaaatatggggttttgggaagattttgggctgacttttttgttattccattttttctcttaaacaaatttttatataataatattatcttgttggcgttattaaaaaagataataatttttttttttatctttgttatattattgctgtcattgaaaaatataaatatggtttgttgagtttgttacatttgaaagtcacatatacaatatatttatacttataattttaatctattttctttcaaaaaattatataaaatatcgtttgtaatttaaatgcaaaatttctataaaatatcgttgataatctacatgttttaacacaataaaaaagcagaaaaatgagcacgtgagtgcccgtcttttcaccaataataataatatatttttttttatggaataatattaataatatgatgatcgtaaaaataactatttctatattttatgtttgtaacccgaagcagacatctttgacgtagttcaattataattttactttctcaaccccttgcttggtcttaaaaggccaataatgttcatgagttcaacatgtctttatcaggcaaaatactcaaatttcttaatctagctccctgacaagttagtatatgtgattgcataattttttttcatttcacagaatataccatatgatttcctcttctatataattaaagaaagtaattagaaaattttctatccaatcaaataaagttagagaaactgattcaaaatggaaaagtaagagaagttaaactttatcattttattccttttcattctattttctttccctaatcttttcctctcattttcgttccttaatcttattactttacattttgtttgggttgatgcagcttccttgaatttgaggtggacatacatagtgagagtgaagtgatggtagctttggtaaaagttgataacgaaaaataaacgacaagcactagagtcagttaggtatcatgttatgtctatgcattcattatttgcctatcgtatgcttctgttctatgaattatgattgtccgtactgatcgtgtctttcaatctttcttgtgacattgacgaattcagttagtttttcaataagttaatttcaaaaatcttttttatgcagatgcaatagacaccaacatagttaagagaatccaataaacaattgaaatatttggataacaatttatgattttgacaaactcaagaatttcatatgccgacatcaacacatttgataaagagatttgcagcattttcatttatgaaaaagatcatttgcatcaacatccgacaaatccttatgagaaaactaaacatcaaagtctgtataaggtttttctaattcatttttatccggtaatttcaacattctgaatagacctctattttttattttatggttctaatttaagacttattttttgaagtctaggtcagtcttcttcttcgtatgtctcaaatacgactctgacgttgccatctctgttaacgacgttggttgccggtcctttaacggtaggcgagtacatatttcggtaatcaatttttattcactcaacttctctcctttcattcgttttgaacaaagaatgtaaacacatagggccaacaaatagtgttagattaggagcttgttcacatctaaggtaaaaactttaatcacaagggatattctaatcattaatactaggttccaccaattaataaatgtgcatgtgtagtttaacatttaagcctagtgtcagtgttaagaggtatatatataggtggaatgtagtagcaagttgctggtgtacaatactataatgcagtagtacttactggttgtttggtagatagatactttaaagagtttgcttagcccactgggtggttgttgttgatagttggtgagctgcggtagaatattgtttgttgttagttagttatacagaaaaaatgttacataatttttgcacagatccgaatcttatcttaaatgtatatacgtggtttttattttctgagtttgtatttagaccatctaaacagttctttgcaatttaagatcccacacctaatttattgggattttttttaggtatcttaaaatccagtctcacttaaaacctctatttttaagtggaccctacgccaattttaagaaaataatcattattgagttgcttttgttgaaatataaatgataaagaattattagtggaagtcatttgaacattttgttagaggatctctattaaagtggttgggtaccaattagatactattattaaaaatttataaaaaagttataagtctatgtggaacctgtttcagtcaaatacctttataggttttaaagtttttcttagtatttttatggactttattataagaatctctaagaaaatttacaaatattaagaaaaattattaaaataatatatttgcatgtgtgtcactattaataaattattatttttgtgtatccatgtagaaaagagtaataagtacattaaaaaatatatagagttttatagactatatatatatatatatattttttttttttttgataaccgtcttgatattttgttaatcaaaataactcatgtcgcaacagtaaaaagaaaaggggaaaaagaaccaaagttggaaatattgtgaagtgcaatgaagtaatacgattaaaacttgaaagaacattgaacacgtaaaattaaatagtgagaaatggtaaagattatcttcaaaacacttgttaaaaaattaaaataggaatattttcttgaaaattagtaaaaaaaagtgtatattcaattttttcgaatgtctaaaaaattattgtttctctaatataaaagggttgcaatccaaacccccgtgactactatttcttcttcttaaatatcatgaatccgtgttttgtttcgtgttctttttttggctacataagaaaaagaaaaaagaaaggaaaaaacggaaagaaaaactaacccttgaaaaggacacgcctaaagcatcacctagtgaagcatgtgataaacacggaagaggctcactaaatgtaacaagatagcctgaacttttagctcccaacttaacaaggatatcgactcaagagtttccatcccaaaggatatgctgaatagaaataaaccaatccttatcaagataattctggatgagttccaagagatttgcataatgatgaaacctcgagatgtccttcatcaccaactctaccacatgaatagagtcgaagaaacacataagctttctataacctgtctcccaacataactttataccaatcaacgtttgaatctcagcatgtaaaatatttgagatccccacgctaccaaataaaccacgaaggaaacttccatcgtgcttacgtattaacccaccatagttcgcctttcctgaattgttcaaagcacttccatcatcattgagaattaaggtgtcctcgtccccatgctgccaactcaccatccttggagtgggaataagagtcactaatgagtaccaaaagctttgtgtgcaagatgaagttgggaaaatattgtcgcgacaatcgagtgtataactagatgaatattgtcaaaaactagtttgtttcttgtacaccaaatattccacataatcaaaggtgcaaggattccaatatccttcactgttttatggattaccttagctatattatccacatccacacctaagcacaatgaaattagaccaggtaaaccacatccctgcaaaaccgcagcagatcgagagcaatggaggagacaatggagaatagtctcatctttacgttttgtgttctaaaaatgtcgattcataaactcatactacttagtgaaaggaaataactctttcaagattttctctaacacttgaaccgaatacatatatatatatatatatatatatatatatatatatatatatatatatatatatatatcttattctcgttcatcactcaacaagtgtatcacttggtctaagtggttattttatgcatcctttcgctaagttgaattgttcaacaccaattgatgccacttttcaagccatatactttattctcatcataaaaaaggtgtataattttttatatacctcatcagaactacttttatttgtatggtgccggtcatcaagtttctacttcaaacatttttagcctctctattaaatagtcaacaatactacatctgaccttaattataagacccttttgaaaaaaaattgcgtccttttttataagactcgtttgttattttcaactatattaatgatttttttacatacatgcccctatttattatacatttttttcttgaatcagcaataaataacatatgaaaaacataaactaactatatctcttaaggataaaattgtaaaaacaatagtgattacaattaactttaatataagtatctactttcttaattctcgtgattttagcaaaagagggacggaagtaatatattttatttgaatggacaactgtatcttttaattttgttgattttgcgatttattttttcttaatcaattacatatggagagaaaaaaaagtggaacttctatccacaccacgcataacaaaaatactaaactaattatgaaatgtgaatacatgaataaataaaaatttataaccatttgatatacaagggatgcccagtattctcatctgtaaatcaacatcaacataatgcacaacaacctaacagggtaatgtcaaaaaataaaaataaaaattttgaaatgcaatagtgcaatgcatagacgacacttgaaaaatcaacatttactcctcatttttttgaacattttttctgaaaaataaacatttttttgaacaagtatacttggaaacatagatttaggtaacctttactcctcattatgtcaggtaaagattcggccttctcttttgatctagaatgaacatatgcattcaagagggcaccatatgtcctcttgtcttttaaatcattggctaggttcagaacaaaaaccttctgcgcttgaaattccgtgaactcttgcgattagatctaactgaatcacagcgtcacttgcatatattttaaacctctctggtctattgttcatccaatcatacacctgcaaaacatgctcattagttattacttttataagatcgttcgtggctgatattaagacatttgtggcaaggaagtatatttaatcagaaaatgtcatactttcatatacattcaaaaggaagcattctgtttaattgaatcgtagaagaaatggacttctaaattattcaaactttactgacaatgttttggaatttacaagaggatagtataagaatggtgctacagtttttttatttgaccaacaatttgttagaggataattatcatgtggagaatatctcctgtagttattgtttagatagcaacaaattatatagttgactcatgacaatcaattgtcattcttagtcattacagtatagttgactcacatgaatcaattgcaattcttagggtgtgtttgtttcaagtttaccaaatttattcctaggaataacattccttggaatataaagatgggaattttattccaaggaatttaggaaaaatatgtttggttagctttataatattcctaggaaccataaaaatagggattataataggtaaattatttatgaatttattcccatcttcatgggaactttattcccacccttttccttgggaaattttttctattcccaggaacattttatacccgagaataaaatttagtaaacttgtaccaaacataggcatatacattccaatcattttattcccgggaatcaacaattataacttgaaacaaacacacccttagtcattatgcaattgatattcattcctagtttatagtatcagtattgtgtatatatatagatgtaacagatgttcataatatattatcaattcaaaatcaacacagtattgctgttactattatatggtatcagcctctccctaacttaacaacttcacgatccaatttttttttacctatcatgacttccgcaacttccacaaactcccccaacaattccaactcatttcaggttggcactcatactgtcattgttctcaacactcaaaactcaatcaaactcaagaacaacaactatccagcatgacatattcaaatgaatgcactattcattggatatgacctcacaggttttatcaatggtacaaaattgtgccctgccaaaacagatccagattacaactactggacaagacaggaccaactcatactacatgctattatcacatctgtcgatcatagcatcatcacgacattaggcaatgtcaaatcctcccaacaagctt

(19) United States
(12) Patent Application Publication
(10) Pub. No.: US 2011/0265225 A1
(43) Pub. Date: Oct. 27, 2011

(54) TRANSGENIC PLANT HAVING INCREASED SEED SIZE

(75) Inventors: Takeshi Yoshizumi, Kanagawa (JP); Minami Matsui, Kanagawa (JP); Tomoko Kuriyama, Kanagawa (JP); Hirofumi Kuroda, Kanagawa (JP); Shinya Takahashi, Kanagawa (JP); Hirohiko Hirochika, Ibaraki (JP)

(73) Assignees: NATIONAL INSTITUTE OF AGROBIOLOGICAL SCIENCES, TSUKUBA-SHI (JP); RIKEN, WAKO-SHI (JP)

(21) Appl. No.: 13/124,544

(22) PCT Filed: Oct. 16, 2009
(86) PCT No.: PCT/JP09/67875

§ 371 Date: Jul. 11, 2011

(30) Foreign Application Priority Data
Oct. 16, 2008 (JP) 2008-267877

(51) Int. Cl. C12N 15/82; C12N 15/63; A01H 5/10; A01H 5/00; C12N 5/10

(52) U.S. Cl. 800/290; 800/298; 435/419; 435/320.1

(57) ABSTRACT

The present invention relates to a transgenic plant having increased seed size prepared via introduction of DNA encoding a protein consisting of a specific amino acid sequence derived from a plant, such as rice, and a method for producing the same.

### TECHNICAL FIELD

The present invention relates to a transgenic plant having increased seed size and a method for producing the same.

### BACKGROUND ART

In order to increase plant seed size, cross breeding and random mutagenesis of genes of interest have heretofore been carried out. However, no satisfactory mutagenesis techniques of target genes of plants have yet been established. Thus, artificial modification has been difficult. Although seed size-related genes were isolated, most of such genes were identified as genes regulating seed size because seed sizes were increased or decreased when such genes were destroyed.

An example of a transgenic plant into which a gene increasing seed size has been introduced is a transgenic plant having increased seed weight resulting from introduction of the rice-derived PLASTOCHRON1 gene (Accession Number of the National Center for Biotechnology Information (NCBI): AB096259) with the use of a binary vector via electroporation into a rice plant (JP Patent Publication (kokai) No. 2005-204621 A). Such transgenic plant was prepared by introducing a rice-derived gene into a rice plant. When such gene is introduced into a plant other than rice, however, whether or not seed size could be increased remains unknown.

### DISCLOSURE OF THE INVENTION

### Objects to be Attained by the Invention

The objects of the present invention are to identify DNA related to increased seed size and to provide a transgenic plant having seed size that is increased via introduction of such DNA into a plant and a method for producing the same.

### Means for Attaining the Objects

The present inventors have conducted concentrated studies in order to attain the above objects. As a result, they identified a gene related to increased seed size with the utilization of the Fox hunting system, introduced such gene into a plant, and allowed such gene to overexpress therein, thereby completing the present invention.

The present invention is summarized as follows.

[1] A transgenic plant into which DNA encoding any of proteins (a) to (c) below has been introduced so as to be capable of being expressed therein:

(a) a protein comprising the amino acid sequence as shown in SEQ ID NO: 2, 4, 6, or 8;

(b) a protein comprising an amino acid sequence derived from the amino acid sequence as shown in SEQ ID NO: 2, 4, 6, or 8 by deletion, substitution, or addition of one or several amino acids and having activity of increasing seed size; or

(c) a protein comprising an amino acid sequence having 90% or higher identity with the amino acid sequence as shown in SEQ ID NO: 2, 4, 6, or 8 and having activity of increasing seed size.

[2] A transgenic plant into which any of DNAs (d) to (g) below has been introduced so as to be capable of being expressed therein:

(d) DNA comprising the nucleotide sequence as shown in SEQ ID NO: 1, 3, 5, or 7;

(e) DNA comprising a nucleotide sequence derived from the nucleotide sequence as shown in SEQ ID NO: 1, 3, 5, or 7 by deletion, substitution, or addition of one or several nucleotides and encoding a protein having activity of increasing seed size;

(f) DNA comprising a nucleotide sequence having 90% or higher identity with the nucleotide sequence as shown in SEQ ID NO: 1, 3, 5, or 7 and encoding a protein having activity of increasing seed size; or

(g) DNA hybridizing, under stringent conditions, to DNA comprising a nucleotide sequence complementary to DNA consisting of the nucleotide sequence as shown in SEQ ID NO: 1, 3, 5, or 7 and encoding a protein having activity of increasing seed size.

[3] A transgenic plant into which DNA encoding any of proteins (h) to (j) below has been introduced so as to be capable of being expressed therein:

(h) a protein comprising the amino acid sequence as shown in SEQ ID NO: 9, 10, or 11;

(i) a protein comprising an amino acid sequence derived from the amino acid sequence as shown in SEQ ID NO: 9, 10, or 11 by deletion, substitution, or addition of one or several amino acids and having activity of increasing seed size; or

(j) a protein comprising an amino acid sequence having 90% or higher identity with the amino acid sequence as shown in SEQ ID NO: 9, 10, or 11 and having activity of increasing seed size.

[4] The transgenic plant according to any of [1] to [3], wherein the activity of increasing seed size is activity of increasing surface area, volume, or weight of seed.

[5] The transgenic plant according to any of [1] to [4], which is a plant, part of a plant, cultured plant cell, or seed.

[6] A recombinant vector comprising DNA encoding any of proteins (a) to (c) below:

(a) a protein comprising the amino acid sequence as shown in SEQ ID NO: 2, 4, 6, or 8;

(b) a protein comprising an amino acid sequence derived from the amino acid sequence as shown in SEQ ID NO: 2, 4, 6, or 8 by deletion, substitution, or addition of one or several amino acids and having activity of increasing seed size; or

(c) a protein comprising an amino acid sequence having 90% or higher identity with the amino acid sequence as shown in SEQ ID NO: 2, 4, 6, or 8 and having activity of increasing seed size.

[7] A recombinant vector comprising any of DNAs (d) to (g) below:

(d) DNA comprising the nucleotide sequence as shown in SEQ ID NO: 1, 3, 5, or 7;

(e) DNA comprising a nucleotide sequence derived from the nucleotide sequence as shown in SEQ ID NO: 1, 3, 5, or 7 by deletion, substitution, or addition of one or several nucleotides and encoding a protein having activity of increasing seed size;

(f) DNA comprising a nucleotide sequence having 90% or higher identity with the nucleotide sequence as shown in SEQ ID NO: 1, 3, 5, or 7 and encoding a protein having activity of increasing seed size; or

(g) DNA hybridizing, under stringent conditions, to DNA comprising a nucleotide sequence complementary to DNA consisting of the nucleotide sequence as shown in SEQ ID NO: 1, 3, 5, or 7 and encoding a protein having activity of increasing seed size.

[8] A recombinant vector comprising DNA encoding any of proteins (h) to (j) below:

(h) a protein comprising the amino acid sequence as shown in SEQ ID NO: 9, 10, or 11;

(i) a protein comprising an amino acid sequence derived from the amino acid sequence as shown in SEQ ID NO: 9, 10, or 11 by deletion, substitution, or addition of one or several amino acids and having activity of increasing seed size; or

(j) a protein comprising an amino acid sequence having 90% or higher identity with the amino acid sequence as shown in SEQ ID NO: 9, 10, or 11 and having activity of increasing seed size.

[9] A method for producing a transgenic plant comprising introducing DNA encoding any of proteins (a) to (c) below into a plant cell and cultivating a plant:

(a) a protein comprising the amino acid sequence as shown in SEQ ID NO: 2, 4, 6, or 8;

(b) a protein comprising an amino acid sequence derived from the amino acid sequence as shown in SEQ ID NO: 2, 4, 6, or 8 by deletion, substitution, or addition of one or several amino acids and having activity of increasing seed size; or

(c) a protein comprising an amino acid sequence having 90% or higher identity with the amino acid sequence as shown in SEQ ID NO: 2, 4, 6, or 8 and having activity of increasing seed size.

[10] A method for producing a transgenic plant comprising introducing any of DNAs (d) to (g) below into a plant cell and cultivating a plant:

(d) DNA comprising the nucleotide sequence as shown in SEQ ID NO: 1, 3, 5, or 7;

(e) DNA comprising a nucleotide sequence derived from the nucleotide sequence as shown in SEQ ID NO: 1, 3, 5, or 7 by deletion, substitution, or addition of one or several nucleotides and encoding a protein having activity of increasing seed size;

(f) DNA comprising a nucleotide sequence having 90% or higher identity with the nucleotide sequence as shown in SEQ ID NO: 1, 3, 5, or 7 and encoding a protein having activity of increasing seed size; or

(g) DNA hybridizing, under stringent conditions, to DNA comprising a nucleotide sequence complementary to DNA consisting of the nucleotide sequence as shown in SEQ ID NO: 1, 3, 5, or 7 and encoding a protein having activity of increasing seed size.

[11] A method for producing a transgenic plant comprising introducing DNA encoding any of proteins (h) to (j) below into a plant cell and cultivating a plant:

(h) a protein comprising the amino acid sequence as shown in SEQ ID NO: 9, 10, or 11;

(i) a protein comprising an amino acid sequence derived from the amino acid sequence as shown in SEQ ID NO: 9, 10, or 11 by deletion, substitution, or addition of one or several amino acids and having activity of increasing seed size; or

(j) a protein comprising an amino acid sequence having 90% or higher identity with the amino acid sequence as shown in SEQ ID NO: 9, 10, or 11 and having activity of increasing seed size.

[12] The method according to any of [9] to [11], wherein DNA is introduced with the use of the recombinant vector according to any of [6] to [8].

The contents as disclosed in the description and/or drawings of Japanese Patent Application No. 2008-267877, to which the present application claims priority, are incorporated herein.

### Effects of the Invention

DNA used in the present invention is related to increased seed size, and introduction of such DNA into a plant and overexpression therein result in the production of a plant having increased seed size. Since such DNA can be expressed at a high level in a plant if a transformation technique has been established for the plant, the present invention is applicable to any plant. DNA used in the present invention is capable of increasing seed size even if it is introduced into a plant belonging to a different family. Also, the transgenic plant of the present invention enables the production of crops exhibiting higher seed production. By increasing seed size, the commodity value of a plant can be enhanced.

### BRIEF DESCRIPTION OF THE DRAWINGS

FIG. 1 shows a scheme of the examples.

FIG. 2 shows wild-type seeds and T2 seeds of K06835 and K15507.

FIG. 3 shows wild-type seeds and T2 seeds of K32722.

FIG. 4 shows wild-type seeds and T2 seeds of K42340.

FIG. 5 shows the area of wild-type seeds and the area of T2 generation seeds of K32722 and K42340.

FIG. 6 shows histograms showing the area of wild-type seeds and T2 generation seeds of K32722.

FIG. 7 shows histograms showing the area of wild-type seeds and T2 generation seeds of K42340.

FIG. 8 shows the volumes of wild-type seeds and T2 generation seeds of K32722.

FIG. 9 shows a comparison of amino acid sequences (corn, Arabidopsis thaliana, and alfalfa) of proteins having high identity (i.e., homologous proteins) with the amino acid sequence encoded by DNA (AK073303) introduced into K32722.

### BEST MODE FOR CARRYING OUT THE INVENTION

### (1) DNA Related to Increased Seed Size

DNA related to increased seed size used in the present invention encodes any of proteins (a) to (c) below:

(a) a protein comprising the amino acid sequence as shown in SEQ ID NO: 2, 4, 6, or 8;

(b) a protein comprising an amino acid sequence derived from the amino acid sequence as shown in SEQ ID NO: 2, 4, 6, or 8 by deletion, substitution, or addition of one or several amino acids and having activity of increasing seed size; or

(c) a protein comprising an amino acid sequence having 90% or higher identity with the amino acid sequence as shown in SEQ ID NO: 2, 4, 6, or 8 and having activity of increasing seed size.

Alternatively, DNA related to increased seed size used in the present invention is any of DNAs (d) to (g) below:

(d) DNA comprising the nucleotide sequence as shown in SEQ ID NO: 1, 3, 5, or 7;

(e) DNA comprising a nucleotide sequence derived from the nucleotide sequence as shown in SEQ ID NO: 1, 3, 5, or 7 by deletion, substitution, or addition of one or several nucleotides and encoding a protein having activity of increasing seed size;

(f) DNA comprising a nucleotide sequence having 90% or higher identity with the nucleotide sequence as shown in SEQ ID NO: 1, 3, 5, or 7 and encoding a protein having activity of increasing seed size; or

(g) DNA hybridizing, under stringent conditions, to DNA comprising a nucleotide sequence complementary to DNA consisting of the nucleotide sequence as shown in SEQ ID NO: 1, 3, 5, or 7 and encoding a protein having activity of increasing seed size.

Alternatively, DNA related to increased seed size used in the present invention encodes any of proteins (h) to (j) below:

(h) a protein comprising the amino acid sequence as shown in SEQ ID NO: 9, 10, or 11;

(i) a protein comprising an amino acid sequence derived from the amino acid sequence as shown in SEQ ID NO: 9, 10, or 11 by deletion, substitution, or addition of one or several amino acids and having activity of increasing seed size; or

(j) a protein comprising an amino acid sequence having 90% or higher identity with the amino acid sequence as shown in SEQ ID NO: 9, 10, or 11 and having activity of increasing seed size.

The term “protein (or DNA) having activity of increasing seed size” used herein refers to a protein (or DNA) that is directly or indirectly related to an increase in seed size.

The term “DNA” used herein encompasses genomic DNA, a gene, cDNA, and the like.

The term “activity of increasing seed size” used herein refers to any activity, provided that such activity makes seed size larger than that of wild-type seeds. Examples thereof include activity of increasing surface area, volume, or weight of seed. Seed size is not particularly limited, provided that seeds are larger than wild-type seeds at statistically significant levels. For example, it is preferable that the seeds be larger than wild-type seeds by at least 5%, 10%, 15%, 20%, 25%, 30%, 35%, 40%, 45%, or 50%.

The term “an amino acid sequence derived from the amino acid sequence by deletion, substitution, or addition of one or several amino acids” used herein means, for example, that 1 to 10 and preferably 1 to 5 amino acids may be deleted in a given amino acid sequence, that 1 to 10 and preferably 1 to 5 amino acids may be substituted with other amino acids in a given amino acid sequence, or that 1 to 10 and preferably 1 to 5 amino acids may be added to a given amino acid sequence.

The term “identity” used in the expression “an amino acid sequence having 90% or higher identity with the amino acid sequence” herein refers to identity of 90% or higher, preferably 95% or higher, more preferably 98% or higher, and further preferably 99% or higher.

The term “identity” used herein refers to the degree of matching between two amino acid sequences (or nucleotide sequences) aligned so as to maximize the number of identical amino acid residues (or the number of identical nucleotides). Specifically, the identity is represented by the percentage (%) of the number of identical amino acid residues (or the number of identical nucleotides) relative to the total number of amino acid residues (or the total number of nucleotides). The percentage identity can be determined with the use of known algorithms, such as BLAST or FASTA. When gaps are introduced as in the case of FASTA, the number of gaps is included in the total number of amino acid residues (or the total number of nucleotides).

The term “a nucleotide sequence derived from the nucleotide sequence by deletion, substitution, or addition of one or several nucleotides” used herein means, for example, that 1 to 10 and preferably 1 to 5 nucleotides may be deleted in a given nucleotide sequence, that 1 to 10 and preferably 1 to 5 nucleotides may be substituted with other nucleotides in a given nucleotide sequence, or that 1 to 10 and preferably 1 to 5 nucleotides may be added to a given nucleotide sequence.

The term “identity” used in the expression “a nucleotide sequence having 90% or higher identity with the nucleotide sequence” herein refers to identity of 90% or higher, preferably 95% or higher, more preferably 98% or higher, and further preferably 99% or higher.

The term “stringent conditions” used herein refers to conditions under which so-called specific hybrids are formed whereas substantially no non-specific hybrids are formed. Under such stringent conditions, for example, a complementary strand of a nucleic acid having high identity (i.e., DNA consisting of a nucleotide sequence having identity of 90% or higher, preferably 95% or higher, more preferably 98% or higher, and further preferably 99% or higher to a given nucleotide sequence) undergoes hybridization whereas a complementary strand of a nucleic acid having identity lower than such level does not undergo hybridization. More specifically, the sodium salt concentration is 15 to 750 mM, preferably 50 to 750 mM, and more preferably 300 to 750 mM, temperature is 25° C. to 70° C., preferably 50° C. to 70° C., and more preferably 55° C. to 65° C., and the formamide concentration is 0% to 50%, preferably 20% to 50%, and more preferably 35% to 45%. Under stringent conditions, further, a filter that had been subjected to hybridization is then washed at a sodium salt concentration of generally 15 to 600 mM, preferably 50 to 600 mM, and more preferably 300 to 600 mM and temperature of 50° C. to 70° C., preferably 55° C. to 70° C., and more preferably 60° C. to 65° C.

Under stringent conditions, alternatively, hybridization may be carried out in the presence of 2× to 6× sodium chloride/sodium citrate (SSC, 1×SSC is 150 mM sodium chloride and 15 mM sodium citrate; pH 7.0) at room temperature to 40° C., and washing may then be carried out in the presence of 0.1× to 1×SSC (preferably 0.1× to 0.2×SSC) and 0.1% SDS at 50° C. to 68° C. once or a plurality of times.

DNA used in the present invention can be obtained in the form of a nucleic acid fragment via PCR amplification with the use of primers designed based on a DNA sequence encoding the amino acid sequence as shown in SEQ ID NO: 2, 4, 6, 8, 9, 10, or 11 or a DNA sequence as shown in SEQ ID NO: 1, 3, 5, or 7 and a nucleic acid obtained from cDNA library or genomic DNA library as a template. Such DNA can be obtained in the form of a nucleic acid fragment via hybridization using a nucleic acid obtained from the above library or the like as a template and a DNA fragment, which is part of such DNA, as a probe. Alternatively, such DNA may be synthesized in the form of a nucleic acid fragment via various techniques of nucleic acid synthesis known in the art, such as chemical synthesis.

Deletion, substitution, or addition of DNA or amino acid as described above can be carried out by modifying DNA encoding a relevant protein by a technique known in the art. Mutation can be introduced into DNA via, for example, the Kunkel method or the Gapped duplex method. Mutation can be introduced with the use of a mutagenesis kit utilizing site-directed mutagenesis (e.g., Mutant-K (Takara Bio Inc.) or the LA PCR in vitro mutagenesis kit (Takara Bio Inc.)) (Sambrook et al., 1989, Molecular Cloning: Laboratory Manual, 2nd edition, Cold Spring Harbor Laboratory Press; Ausubel et al., 1995, Short Protocols in Molecular Biology, third edition, John Wiley & Sons, Inc.).

DNA used in the present invention was found to have activity of increasing seed size for the first time by measuring the sizes of seeds obtained from a transgenic line with the utilization of the Fox hunting system and identifying cDNA that had been introduced into a line producing seeds significantly larger than wild-type seeds.

The Fox hunting system (i.e., the full-length cDNA over-expressor gene hunting system) is a technique for elucidating gene functions based on changes in traits resulting from introduction and high expression of full-length cDNA in a plant (JP Patent Publication (Saikohyo) 2003-018808 A). In the present invention, rice full-length cDNA is used as full-length cDNA to be introduced into a plant, and an example of a plant into which full-length cDNA can be introduced is, but is not limited to, Arabidopsis thaliana.

Specifically, DNA used in the present invention can be identified with the utilization of the Fox hunting system in the following manner.

Specifically, approximately 13,000 independent rice full-length cDNAs are prepared as a pool having equivalent ratios of the cDNAs (referred to as “normalization”), and the cDNAs are integrated into a T-DNA vector having a regulatory region, such as a promoter, an enhancer (e.g., a transcription enhancer E21 or an omega sequence), and a terminator, and a selection marker such as a drug-resistance gene. The resulting T-DNA vectors are introduced into Agrobacterium, a rice full-length cDNA expression library (referred to as a rice FOX library) is produced, and whether or not such library reflects the entire cDNA distribution is then confirmed. Thereafter, Arabidopsis thaliana is transformed using the Agrobacterium as described above by the floral dipping method to produce transgenic Arabidopsis thaliana lines (rice FOX lines). In the FOX hunting system, only one or two clone(s) is/are introduced per plant even when plants are infected with a library consisting of hundreds of millions of clones. Thus, transgenic lines in which a different clone (or clones) is/are introduced for each plant can be produced.

T1 seeds are collected from the primary recombinant Arabidopsis thaliana (T0) of the approximately 20,000 thus-obtained rice FOX lines, sown on a medium containing antibiotics, and selected. Thus, T1-generation transformants are obtained. The seeds obtained via self-pollination of such transformants are designated as T2 seeds. The T2 seeds (for example, 50 grains per line) are visually observed, and, simultaneously, analyzed with the use of a seed morphology measurement program WINSEEDLE (Regent Instruments) by importing an image showing the seeds as the data into a computer. WINSEEDLE is a program that measures morphology, size, and color of seeds based on images of seeds. This program measures the lengths and the widths of seeds that have been imported into a computer, and the areas thereof are then determined. Wild-type Arabidopsis thaliana seeds are used as controls, and lines from which seeds that are significantly larger than wild-type controls are predominantly produced are selected. The seeds of the selected lines are sown and cultivated, and it is confirmed whether or not a phenotype exhibiting increased seed size has been reproduced. Genomic DNAs are extracted from the seeds that had reproduced the phenotype of interest, and the introduced rice full-length cDNAs are isolated via PCR. Such cDNAs are designated as candidates for DNAs causing increased seed size. The candidate DNAs are introduced into Arabidopsis thaliana again and overexpressed therein, whether or not seed size is increased is confirmed, and the confirmed rice full-length cDNAs are determined to be DNAs having activity of increasing seed size (i.e., DNAs encoding proteins having activity of increasing seed size).

DNAs identified in such a manner are registered with NCBI under the accession numbers indicated below:

AK100982 (DNA sequence: SEQ ID NO: 1; amino acid sequence: SEQ ID NO: 2);

AK099678 (DNA sequence: SEQ ID NO:3; amino acid sequence: SEQ ID NO: 4);

AK073303 (DNA sequence: SEQ ID NO:5; amino acid sequence: SEQ ID NO: 6);

AK071204 (DNA sequence: SEQ ID NO:7; amino acid sequence: SEQ ID NO: 8).

The above amino acid sequences derived from rice are compared with amino acid sequences derived from other plant species, and DNA encoding a highly identical amino acid sequence and encoding a protein having activity of increasing seed size can be used in the present invention. The identity can be inspected by, for example, accessing sequence databases of NCBI or EMBL (The European Molecular Biology Laboratory) with the use of a sequence homology search program such as BLAST or FASTA (e.g., Alteschul, S. F. et al., 1990, J. Mol. Biol. 15: 403-410; Karlin, S, and Altschul S. F., 1990, Proc. Natl. Acad. Sci., U.S.A., 87: 2264-2268).

Examples of amino acid sequences derived from other plant species having high identity with the amino acid sequence of AK073303 include a corn-derived amino acid sequence (SEQ ID NO: 9; NCBI Accession Numbers: ACF86069 (the amino acid sequence) and BT041064 (the DNA sequence)), an Arabidopsis thaliana-derived amino acid sequence (SEQ ID NO: 10; NCBI Accession Numbers: NP#567660 (the amino acid sequence) and NM#118359 (the DNA sequence)), and an alfalfa (Medicago truncatula)-derived amino acid sequence (SEQ ID NO: 11; NCBI Accession Numbers: ABD28483 (the amino acid sequence) and AC148915.2 (the DNA sequence)).

### (2) Recombinant Vector

The recombinant vector of the present invention used for plant transformation can be constructed by introducing DNA encoding any of proteins (a) to (c) and (h) to (j) above or any of DNAs (d) to (g) above into an appropriate vector. As the vector, pBI-based, pPZP-based, pSMA-based, and other vectors, which can introduce target DNA into a plant via Agrobacterium, are preferably used. In particular, a pBI-based binary vector or intermediate vector is preferably used, and examples thereof include pBI121, pBI101, pBI101.2, pBI101.3, and pBIG2113 vectors. A binary vector is a shuttle vector that is replicable in Escherichia coli and Agrobacterium. When a plant is infected with Agrobacterium containing a binary vector, DNA held between border sequences consisting of an LB sequence and an RB sequence on the vector can be integrated into the nuclear DNA of the plant. Examples of other vectors include a pUC-based vector that can directly introduce DNA into a plant, such as pUC18, pUC19, and pUC9 vectors. Further examples of other vectors include plant virus vectors, such as cauliflower mosaic virus (CaMV), bean golden mosaic virus (BGMV), and tobacco mosaic virus (TMV) vectors.

When a binary vector-based plasmid is used, target DNA is inserted between the border sequences (between LB and RB) of the aforementioned binary vector, and the recombinant vector is multiplied in E. coli. Subsequently, the multiplied recombinant vectors are introduced into, for example, Agrobacterium tumefaciens GV3101, C58, LBA4404, EHA101, or EHA105 or Agrobacterium rhizogenes LBA1334 via electroporation or other means, and the target DNA is introduced into a plant using such Agrobacterium.

In order to insert target DNA into a vector, at the outset, a method in which purified DNA is cleaved with an appropriate restriction enzyme and the resulting DNA is then ligated to a vector by inserting it into a restriction enzyme site or a multicloning site of an appropriate vector DNA is employed, for example.

Also, it is necessary to integrate target DNA into a vector in a manner that allows the DNA to exert its function. In this regard, a promoter, an enhancer, a terminator, an origin of replication required when using a binary vector system (such as an origin of replication derived from a Ti or Ri plasmid), a selection marker gene, and the like can be ligated to the vector at a region upstream, inside, or downstream of the target DNA.

The promoter is not necessarily derived from a plant, provided that it is DNA that can function in a plant cell and induce expression in a specific tissue or at a specific developmental stage of a plant. Specific examples thereof include a cauliflower mosaic virus (CaMV) 35S promoter, a promoter of the nopaline-synthase gene, an ubiquitin promoter derived from corn, an actin promoter derived from rice, and a PR protein promoter derived from tobacco.

Examples of the enhancers include an enhancer region containing an upstream sequence of the CaMV 35S promoter, a transcription enhancer E12, and an omega sequence, and such enhancers are used to improve expression efficiency of target DNA.

The terminator may be any sequence that can terminate gene transcription driven by the promoter, and examples thereof include a terminator of the nopaline-synthase (NOS) gene, a terminator of the octopine-synthase (OCS) gene, and a terminator of the CaMV 35S RNA gene.

Examples of the selection marker genes include a hygromycin resistance gene, an ampicillin resistance gene, a neomycin resistance gene, a bialaphos resistance gene, and a dihydrofolate reductase gene.

### (3) Transgenic Plant and Method for Producing the Same

The transgenic plant of the present invention can be produced by introducing DNA encoding any of proteins (a) to (c) and (h) to (j) above, any of DNAs (d) to (g) above, or the aforementioned recombinant vector into a target plant. In the present invention, “introduction of DNA” means a situation in which target DNA is introduced into the aforementioned host plant cell by, for example, a known genetic engineering method so as to be capable of being expressed therein. The DNA thus introduced can be integrated into genomic DNA of a host plant, or it can remain contained within an exogenous vector.

The condition “so as to be capable of being expressed” used herein is a condition under which DNA can be constitutively or inductively expressed in the presence of a regulatory sequence, such as a promoter or enhancer.

Introduction of DNA or a recombinant vector may be carried out by a known method, such as an Agrobacterium method, a PEG-calcium phosphate method, electroporation, a liposome method, a particle gun method, or a microinjection method. The Agrobacterium method includes a method using a protoplast, a method using a piece of tissue, and a method using a plant body (the in planta method). The method using a protoplast can be carried out as a method in which a protoplast is co-cultured with Agrobacterium containing a Ti plasmid or an Ri plasmid (Agrobacterium tumefaciens or Agrobacterium rhizogenes, respectively) and a method in which a protoplast is fused with a spheroplast of Agrobacterium (a spheroplast method). The method using a piece of tissue can be carried out via, for example, infection of sterile cultured leaf discs of a target plant or infection of calluses (undifferentiated cultured cells). Also, the in planta method using a seed or a plant body can be carried out by directly treating an imbibed seed, a young plant (a young seedling), a potted plant, and the like with Agrobacterium. Such plant transformation methods can be carried out according to the description in, for example, “Shinpan Model shokubutsu no jikken protocol, Idengakutekishuho kara genome kaiseki made” (literal translation: “New edition, Experimental protocol for model plant, From genetic technique to genome analysis”) (Supervised by Ko Shimamoto and Kiyotaka Okada, Shujunsha Co., Ltd., 2001).

Whether or not DNA is integrated into the plant body can be confirmed via, for example, PCR, Southern hybridization, or Northern hybridization. For example, DNA is prepared from the transgenic plant, primers specific to the target DNA are designed, and PCR is carried out. Subsequently, the amplified product is subjected to agarose electrophoresis, polyacrylamide gel electrophoresis, or capillary electrophoresis and then stained with ethidium bromide, an SYBR Green solution, or the like, so that the amplified product is detected as a single band. Thus, transformation can be confirmed. Also, PCR can be carried out using a primer that has been labeled with a fluorescent dye or the like in advance, and the amplified product can then be detected. Further, transformation may also be confirmed by a method in which the amplified product is bound to a solid phase such as a microplate, following which the amplified product is detected by a fluorescence, enzymatic, or other reaction.

Alternatively, transformation can also be confirmed by producing a vector having one of various reporter genes, such as a gene for β-glucuronidase (GUS), luciferase (LUC), green fluorescent protein (GFP), chloramphenicol acetyltransferase (CAT), or β-galactosidase (LacZ) ligated to a region downstream of the target DNA, transforming a plant using Agrobacterium into which the vector was introduced as described above, and then measuring the expression level of the reporter gene.

Plants to be used for transformation in the present invention can be either monocotyledons or dicotyledons, and examples thereof include, but are not limited to, plants belonging to the family Brassicaceae (including Arabidopsis thaliana, cabbage, and rapeseed), the family Leguminosae (including garden pea, soybean, alfalfa, Phaseolus angularis, horse bean, and Vigna sinensis), the family Poaceae (including rice, corn, barley, and wheat), the family Compositae (including safflower and sunflower), the family Pedaliaceae (e.g., sesame), and the family Euphorbiaceae (including Jatropha curcas and Ricinus communis). Seed plants, such as edible seed plants and seed plants for oil extraction, are particularly preferable.

In the present invention, a plant material to be subjected to transformation may be any plant organ or tissue, such as a stem, a leaf, a seed, an embryo, an ovule, an ovary, a shoot apex, an anther, and pollen, sections of the above plant organs and tissues, an undifferentiated callus, and a cultured plant cell such as a protoplast obtained by removing the cell wall of the callus by enzymatic treatment. When the in planta method is employed, an imbibed seed and an entire plant body can also be used.

When a cultured plant cell is subjected to transformation, an organ or an organism may be regenerated by a known tissue culture method in order to regenerate a transformant from the resulting transformed cell. A person skilled in the art can easily perform the procedure as described above using a generally known method for regenerating a plant body from a plant cell. For example, regeneration of a plant body from a plant cell can be performed as follows.

When a plant tissue or protoplast is used as a plant material to be subjected to transformation, the plant tissue or protoplast is cultured in a medium for callus formation, which is prepared by adding inorganic elements, vitamins, carbon sources, sugars as energy sources, plant growth regulators (plant hormones, such as auxin, cytokinin, gibberellin, abscisic acid, ethylene, and brassinosteroid), and the like, followed by sterilization. The plant tissue or protoplast is then allowed to form a dedifferentiated callus, which will proliferate into an amorphous mass (hereinafter referred to as “callus induction”). The callus thus formed is transferred to a fresh medium containing plant growth regulators, such as auxin, and is subjected to further proliferation (subculture).

When callus induction is performed on a solid medium such as agar and subculture is performed via liquid culture, for example, each culture can be efficiently carried out in a large amount. Subsequently, callus that has proliferated via subculture is cultured under appropriate conditions to induce re-differentiation of an organ (hereinafter referred to as “re-differentiation induction”), and a complete plant body is regenerated in the end. The re-differentiation induction can be carried out by appropriately setting the types and the amounts of constituents in the medium, such as plant growth regulators such as auxin, as well as carbon sources, light, temperature, and other conditions. An adventitious embryo, an adventitious root, an adventitious shoot, an adventitious stem, an adventitious leaf, and the like are formed, and these adventitious organs further grow into a complete plant body via re-differentiation induction. Alternatively, such adventitious organs can be stored in the form before they grow into a complete plant body (in the form of an encapsulated artificial seed, dried embryo, or lyophilized cell or tissue, for example).

The transgenic plant of the present invention encompasses any of an entire plant body, a part of a plant body (e.g., a leaf, a petal, a stem, a root, and pollen), a cultured plant cell (e.g., a callus and a protoplast), and a seed into which DNA encoding any of proteins (a) to (c) and (h) to (j) above or any of DNAs (d) to (g) above have been introduced. Further, it encompasses progeny plant bodies obtained via sexual or asexual reproduction of plant body and parts, cultured cells, and seeds of the progeny plant body. The transgenic plant of the present invention can be produced in large quantities by obtaining a reproductive material, such as a seed and a protoplast, from the transgenic plant and cultivating or culturing the reproductive material.

### EXAMPLES

Hereafter, the present invention is described in greater detail with reference to the following examples, although the present invention is not limited thereto.

### (1) Production of Rice FOX Line by FOX Hunting System

In the present example, pBIG2113SF, which was obtained by introducing an SfiI cloning site into a constitutive expression vector, pBIG2113N (Taji, T. et al., Plant J., 2002, 24 (4): pp. 417-426; and Becker, D. et al., Nucleic Acid Res., 1990, 18 (1): p. 203), was used as a vector below.

(i) Production of Normalized Rice Full-Length cDNA Mix

Full-length cDNA was prepared from rice by the CAP trapper method. The resulting cDNA was cloned into a site between SfiI restriction enzyme sites in Lambda ZAP or Lambda pLC-1-B (reference: Seki, M. et al., Plant J., 15, 707-720, 1998). Sequences at the 5′ end and the 3′ end of cDNA were sequenced using the vector sequence, cDNAs were grouped, and 20,000 independent clones were identified (reference: Seki, M. et al., Plant Physiol. Biochem., 39, 211-220, 2001). Subsequently, a 0.5-μl aliquot was taken from each clone prepared at 50 ng/μl, and all the aliquots were mixed in a single tube. One microliter of the mixture was used to transform 20 μl of the electric competent cell DH10B (Gibco BRL, U.S.A.). Approximately 200,000 independent colonies grown on agar media containing Amp were mixed, and plasmids were collected therefrom. The plasmids thus obtained were designated as the normalized rice full-length cDNA mix.

### (ii) Production of Rice FOX Library

The normalized rice full-length cDNA mix (2 μg) and 700 μg of pBIG2113SF were mixed together, and completely cleaved with SfiI simultaneously. Thereafter, the cleaved products were concentrated via isopropanol precipitation. The precipitate was dissolved in 8 μl of water, and mixed with 1 μl of 10× buffer and 1 μl of T4 ligase, and a reaction was allowed to proceed overnight at 16° C. The reaction solution (2 μl) was mixed with 40 μl of the Electric competent cell DH10B, and transformation was performed.

Approximately 150,000 independent colonies grown on agar media containing kanamycin (Km) were mixed, and plasmids were collected therefrom. The plasmid solution thus collected (2 μl) was mixed with 40 μl of the Electric competent Agrobacterium cell GV3101, and transformation was performed. Approximately 150,000 independent colonies grown on agar media containing Km were suspended in LB liquid medium, glycerol was added thereto to prepare a 15% glycerol solution, and the resulting solution was stored at −80° C. The glycerol solution thus obtained was designated as a rice FOX library.

(iii) Production of Rice FOX Line

Approximately 200,000 colonies of the aforementioned rice FOX library were grown and suspended in a dipping solution, and floral dipping of wild-type Arabidopsis thaliana (Colombia (Col-0)) was performed. Seeds (T1 seeds) were harvested and allowed to germinate on nutrient-poor media, BAM, containing hygromycin, and only approximately 20,000 plant lines exhibiting hygromycin resistance were then transplanted to soil. The plants were allowed to self-pollinate, the resulting seeds were designated as T2 seeds, and such seeds were used for seed size observation.

(2) Selection of Rice FOX Line Via Observation of T2 Seeds, Identification of Nucleotide Sequence of Causal DNA, and Reintroduction of DNA into Arabidopsis thaliana

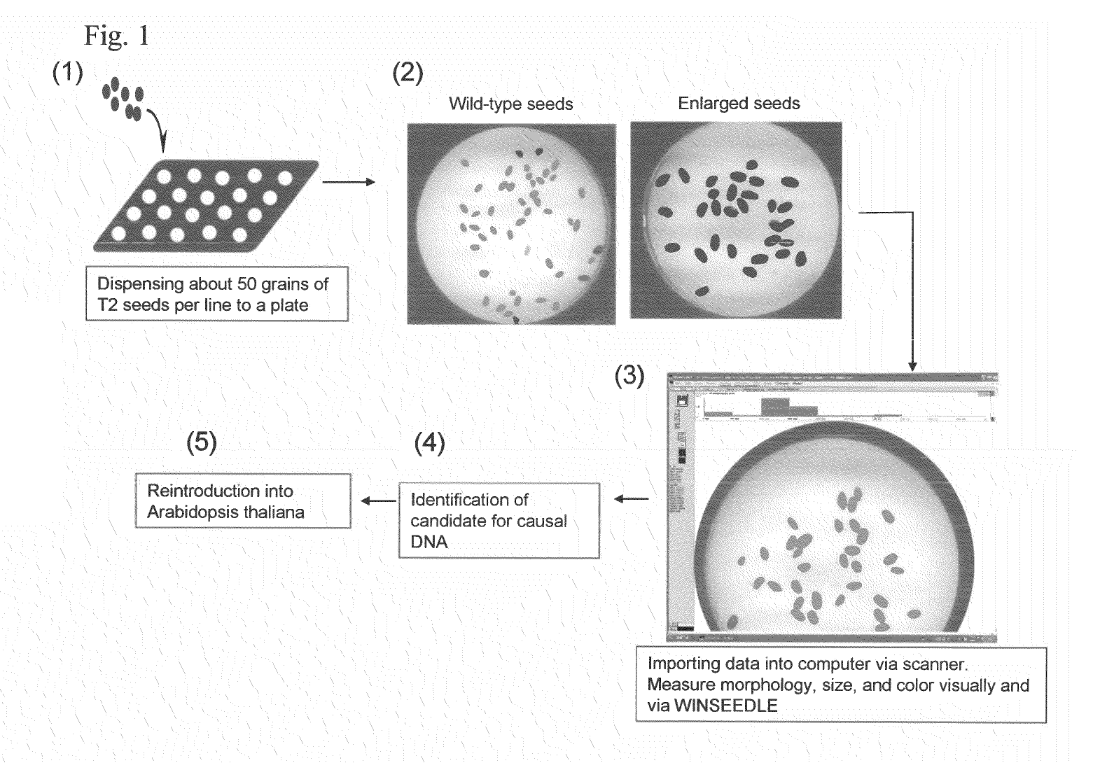

The T2 seeds were placed in wells of a plastic plate in amounts of approximately 50 grains per line (FIG. 1 (1)). While the seeds were visually inspected, an image showing the seeds was imported as the data into a computer (FIG. 1(2)). At this time, lines predominantly producing seeds that were significantly larger than wild-type seeds were selected. The image imported into a computer was analyzed by the WINSEEDLE program by measuring lengths and widths of seeds in the image, and the areas were then determined (FIG. 1(3)). The seeds of the selected lines were sown and cultivated, and it was confirmed whether or not a phenotype exhibiting increased seed size would be reproduced in the subsequent generation. Genomic DNAs were extracted from the seeds that had reproduced the phenotype of interest, the introduced rice full-length cDNAs were isolated via PCR, and the nucleotide sequences were identified.

PCR was carried out using a PCR reaction solution having the composition shown in Table 1, and a cycle of 94° C. for 0.5 minutes, 55° C. for 0.5 minutes, and 72° C. for 4 minutes was repeated 40 times.

TABLE 1
Composition of the reaction solution:

| | | |
|---|---|---|
| Primers (100 pM) | 2 × 0.25 | μl |
| dNTP (200 μM) | 4 | μl |
| Buffer (x2) | 25 | μl |
| Polymerase | 0.5 | μl |
| Genomic DNA | 10 | μl |
| Distilled water | 10 | μl |
| Total | 50 | μl |

The primers used for PCR were as follows:

| | |
|---|---|
| GTACGTATTTTTACAACAATTACCAACAAC; | (SEQ ID NO: 12) |
| GGATTCAATCTTAAGAAACTTTATTGCCAA. | (SEQ ID NO: 13) |

The PCR products were collected from agarose gels, mixed with pBIG2113SF, completely cleaved with SfiI, precipitated by isopropanol, and then treated with T4 ligase. The resultants were then used to transform E. coli. Plasmids into which the PCR fragments had been inserted were selected, and the nucleotide sequences of the cDNA fragments of the inserted candidates for causal DNAs were identified using the aforementioned primers (FIG. 1(4)).

The candidates for causal DNAs were overexpressed again in Arabidopsis thaliana in the same manner as that used for the FOX hunting system (FIG. 1(5)), and whether seed size would be increased was inspected. The confirmed rice full-length cDNA was designated as DNA that increases seed size.

(3) DNA that Increases Seed Size

DNAs that were found to increase seed size in (2) above were those introduced into the four rice FOX lines indicated by the numbers below. The numbers in parentheses indicate the NCBI Accession Numbers and the SEQ ID NOs of the nucleotide sequences used herein of the DNAs introduced into the rice FOX lines.

K06835 (AK100982, SEQ ID NO: 1);

K15507 (AK099678, SEQ ID NO: 3);

K32722 (AK073303, SEQ ID NO: 5);

K42340 (AK071204, SEQ ID NO: 7).

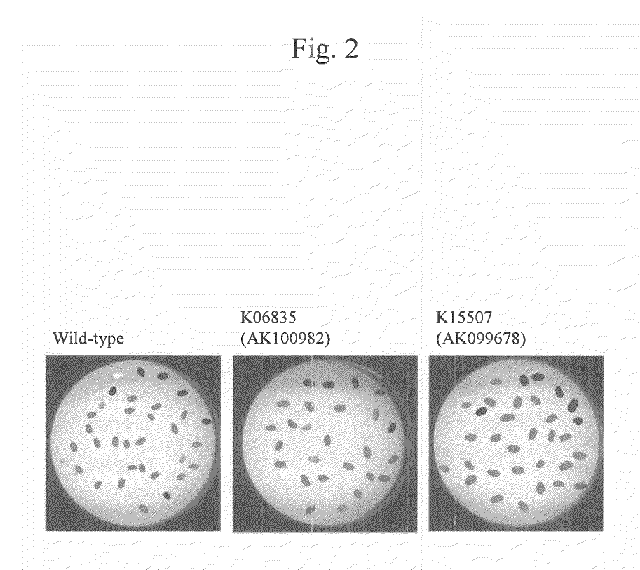

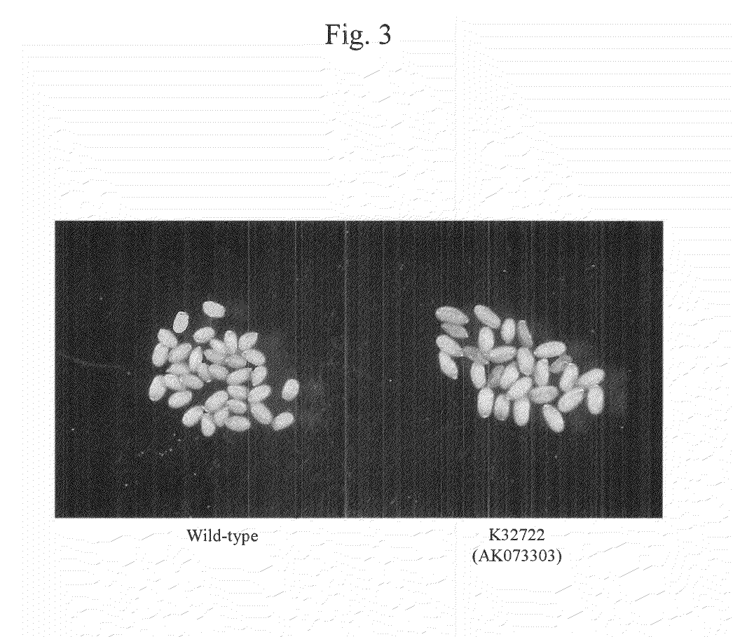

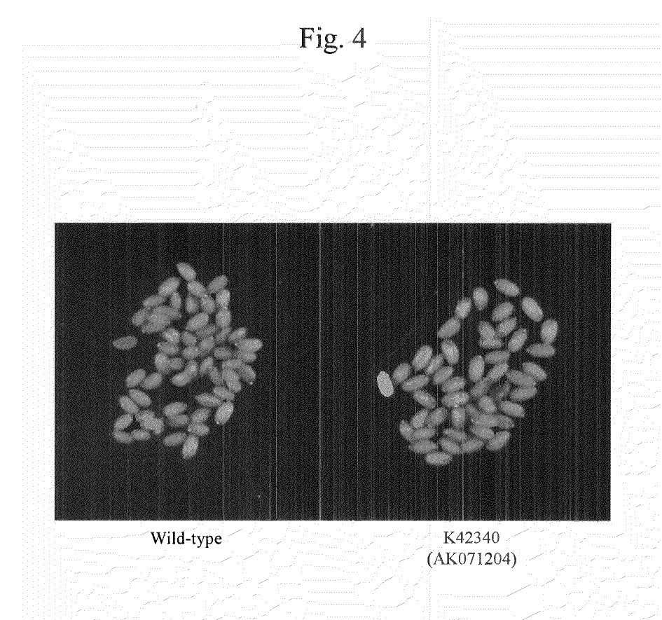

FIG. 2 shows wild-type seeds and T2 seeds of K06835 and K15507. FIG. 3 shows wild-type seeds and T2 seeds of K32722. FIG. 4 shows wild-type seeds and T2 seeds of K42340. Based on the figures, the seeds of the rice FOX lines were found to be larger than wild-type seeds.

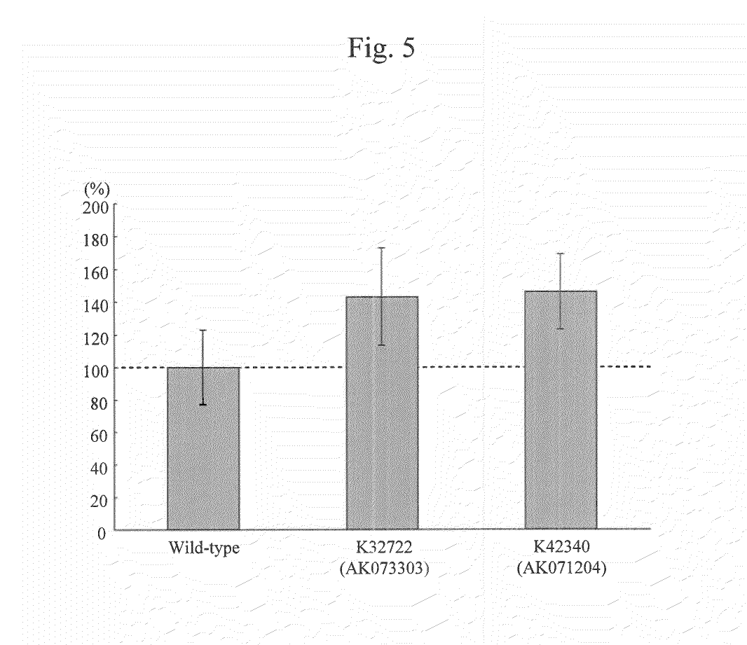

The areas of wild-type seeds were compared with the areas of the seeds (T2 generation seeds) obtained by reintroducing DNAs that had been introduced into K32722 and K42340 in Arabidopsis thaliana and overexpressing the same therein (FIG. 5). Compared with wild-type seeds, the areas of K32722 and K42340 seeds were found to have increased by approximately 40%.

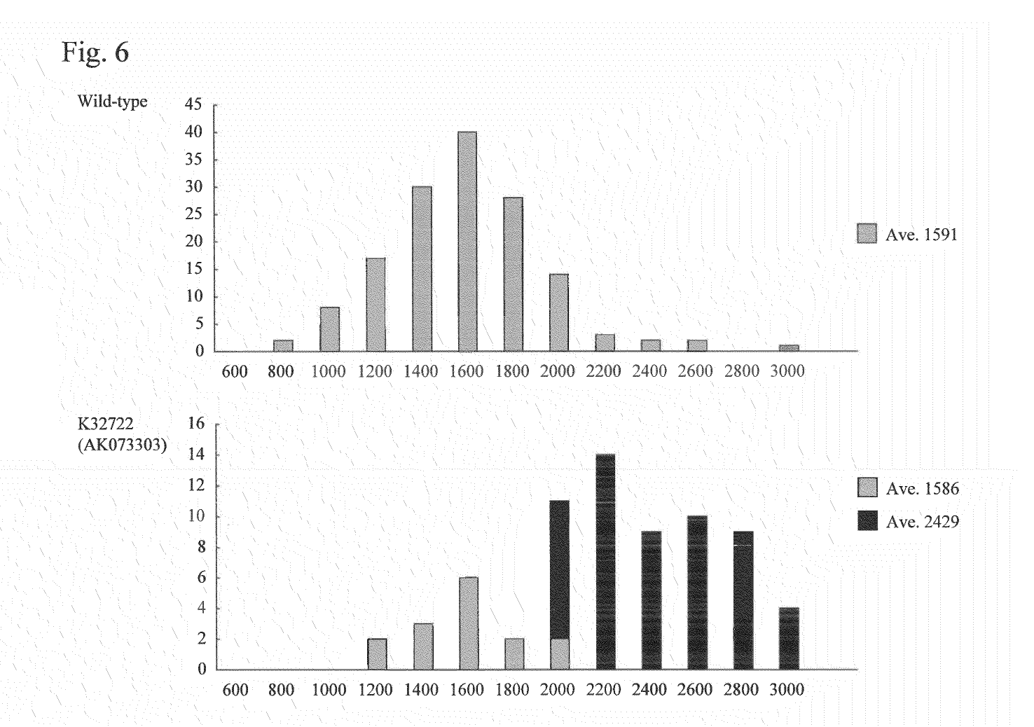

FIG. 6 shows histograms showing the areas of wild-type seeds and T2 generation seeds of K32722 (i.e., distribution of seed areas of K32722-overexpressing lines). The vertical axis indicates the seed number and the horizontal axis indicates the areas (i.e., the pixel numbers). Wild-type seeds exhibited a large peak at a pixel number of 1,600, and such peak has a symmetric normal distribution-like shape. In contrast, the K32722 seeds exhibited a large peak and a small peak. The small peak (gray) appeared at the same position as that for the wild-type seeds (i.e., at a pixel number of 1,600), and the large peak (black) appeared at a position representing areas larger than those of wild-type seeds (i.e., at a pixel number of 2,200). Since the phenotype of the transformant appeared predominantly in the segregating generation, the large peak was considered to represent the transformant. The average area for the seeds constituting the small peak (a pixel number of 1,586) was found to be substantially consistent with that for the wild-type seeds (a pixel number of 1,591), and the average area for the seeds constituting the large peak (a pixel number of 2,429) was found to be greater than that for the wild-type seeds by approximately 50%.

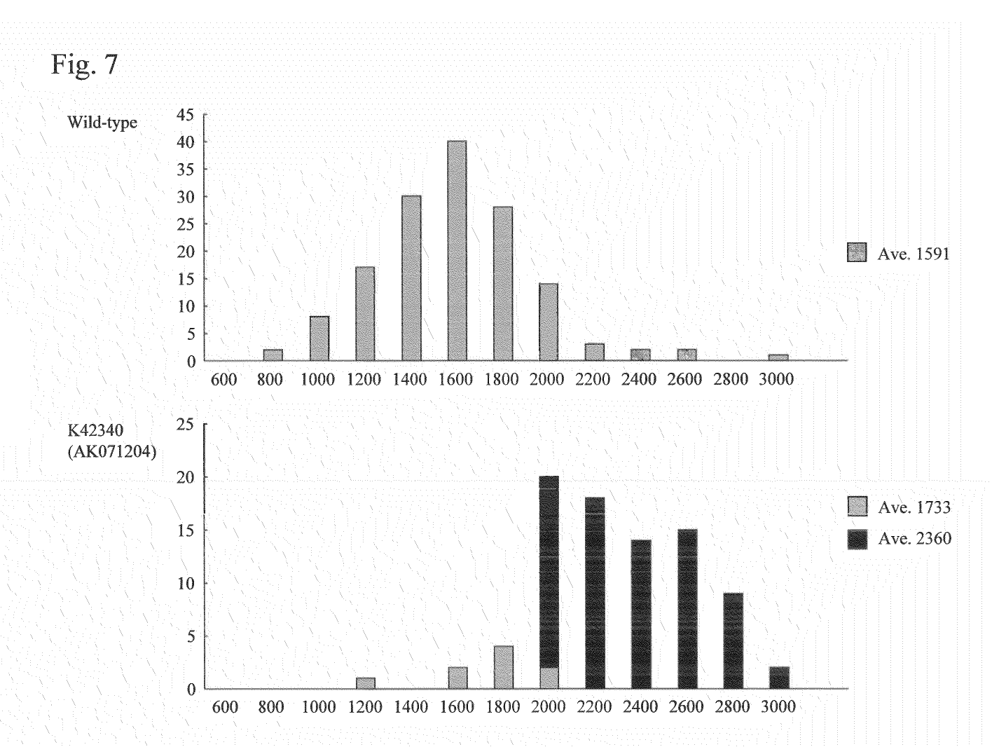

FIG. 7 shows histograms showing the areas of wild-type seeds and T2 generation seeds of K42340 (i.e., distribution of seed areas of K42340-overexpressing lines). The vertical axis indicates the seed number and the horizontal axis indicates the areas (i.e., the pixel numbers). K42340 exhibited an irregular peak. The peak of K42340 seeds was shifted to the right as compared with that of wild-type seeds. This indicates that seeds larger than wild-type seeds are dominant in this population.

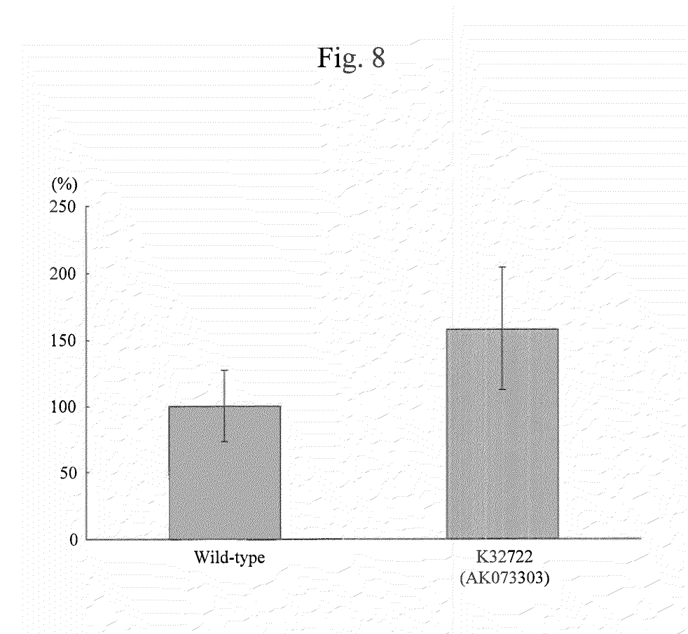

FIG. 8 shows the results of calculation of the volumes as spheroids of T2 generation seeds of K32722 as an example (i.e., the seed volumes of the K32722-overexpressing lines). Compared with wild-type seeds, the volumes of the K32722 seeds increased by approximately 50%.

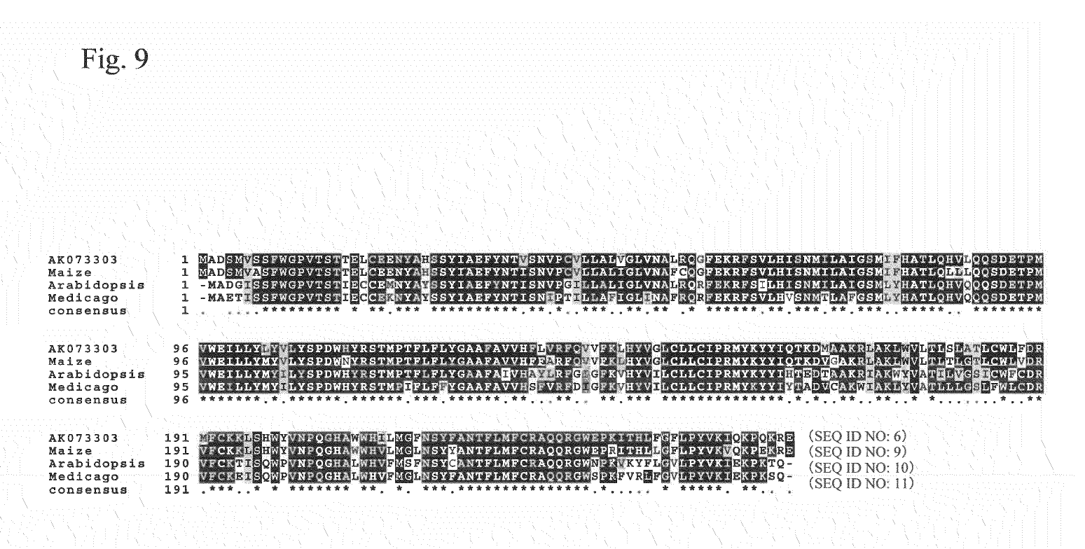

FIG. 9 shows the results of a comparison of the amino acid sequence (SEQ ID NO: 6) of DNA (AK073303) introduced into K32722, the amino acid sequence (SEQ ID NO: 9) of corn (family: Gramineae; genus: Zea), the amino acid sequence (SEQ ID NO: 10) of Arabidopsis thaliana (family: Brassicaceae; genus: Arabidopsis), and the amino acid sequence (SEQ ID NO: 11) of alfalfa (family: Leguminosae; genus: Medicago). The results demonstrate that DNA of AK073303 is highly conserved across plants of different families. It is considered that introduction of DNAs encoding the amino acid sequences as shown in SEQ ID NO: 9, SEQ ID NO: 10, and SEQ ID NO: 11 into plants results in increased seed size.

All publications, patents, and patent applications cited herein are incorporated herein by reference in their entirety.

1. A transgenic plant into which DNA encoding any of proteins (a) to (c) below has been introduced so as to be capable of being expressed therein:
(a) a protein comprising the amino acid sequence as shown in SEQ ID NO: 2, 4, 6, or 8;
(b) a protein comprising an amino acid sequence derived from the amino acid sequence as shown in SEQ ID NO: 2, 4, 6, or 8 by deletion, substitution, or addition of one or several amino acids and having activity of increasing seed size; or
(c) a protein comprising an amino acid sequence having 90% or higher identity with the amino acid sequence as shown in SEQ ID NO: 2, 4, 6, or 8 and having activity of increasing seed size.

2. A transgenic plant into which any of DNAs (d) to (g) below has been introduced so as to be capable of being expressed therein:
(d) DNA comprising the nucleotide sequence as shown in SEQ ID NO: 1, 3, 5, or 7;
(e) DNA comprising a nucleotide sequence derived from the nucleotide sequence as shown in SEQ ID NO: 1, 3, 5, or 7 by deletion, substitution, or addition of one or several nucleotides and encoding a protein having activity of increasing seed size;
(f) DNA comprising a nucleotide sequence having 90% or higher identity with the nucleotide sequence as shown in SEQ ID NO: 1, 3, 5, or 7 and encoding a protein having activity of increasing seed size; or
(g) DNA hybridizing, under stringent conditions, to DNA comprising a nucleotide sequence complementary to DNA consisting of the nucleotide sequence as shown in SEQ ID NO: 1, 3, 5, or 7 and encoding a protein having activity of increasing seed size.

3. A transgenic plant into which DNA encoding any of proteins (h) to (j) below has been introduced so as to be capable of being expressed therein:
(h) a protein comprising the amino acid sequence as shown in SEQ ID NO: 9, 10, or 11;
(i) a protein comprising an amino acid sequence derived from the amino acid sequence as shown in SEQ ID NO: 9, 10, or 11 by deletion, substitution, or addition of one or several amino acids and having activity of increasing seed size; or
(j) a protein comprising an amino acid sequence having 90% or higher identity with the amino acid sequence as shown in SEQ ID NO: 9, 10, or 11 and having activity of increasing seed size.

4. The transgenic plant according to any one of claims 1 to 3, wherein the activity of increasing seed size is activity of increasing surface area, volume, or weight of seed.

5. The transgenic plant according to any one of claims 1 to 4, which is a plant, part of a plant, cultured plant cell, or seed.

6. A recombinant vector comprising DNA encoding any of proteins (a) to (c) below:
(a) a protein comprising the amino acid sequence as shown in SEQ ID NO: 2, 4, 6, or 8;
(b) a protein comprising an amino acid sequence derived from the amino acid sequence as shown in SEQ ID NO: 2, 4, 6, or 8 by deletion, substitution, or addition of one or several amino acids and having activity of increasing seed size; or
(c) a protein comprising an amino acid sequence having 90% or higher identity with the amino acid sequence as shown in SEQ ID NO: 2, 4, 6, or 8 and having activity of increasing seed size.

7. A recombinant vector comprising any of DNAs (d) to (g) below:
(d) DNA comprising the nucleotide sequence as shown in SEQ ID NO: 1, 3, 5, or 7;
(e) DNA comprising a nucleotide sequence derived from the nucleotide sequence as shown in SEQ ID NO: 1, 3, 5, or 7 by deletion, substitution, or addition of one or several nucleotides and encoding a protein having activity of increasing seed size;
(f) DNA comprising a nucleotide sequence having 90% or higher identity with the nucleotide sequence as shown in SEQ ID NO: 1, 3, 5, or 7 and encoding a protein having activity of increasing seed size; or
(g) DNA hybridizing, under stringent conditions, to DNA comprising a nucleotide sequence complementary to DNA consisting of the nucleotide sequence as shown in SEQ ID NO: 1, 3, 5, or 7 and encoding a protein having activity of increasing seed size.

8. A recombinant vector comprising DNA encoding any of proteins (h) to (j) below:
(h) a protein comprising the amino acid sequence as shown in SEQ ID NO: 9, 10, or 11;
(i) a protein comprising an amino acid sequence derived from the amino acid sequence as shown in SEQ ID NO: 9, 10, or 11 by deletion, substitution, or addition of one or several amino acids and having activity of increasing seed size; or
(j) a protein comprising an amino acid sequence having 90% or higher identity with the amino acid sequence as shown in SEQ ID NO: 9, 10, or 11 and having activity of increasing seed size.

9. A method for producing a transgenic plant comprising introducing DNA encoding any of proteins (a) to (c) below into a plant cell and cultivating a plant:
(a) a protein comprising the amino acid sequence as shown in SEQ ID NO: 2, 4, 6, or 8;
(b) a protein comprising an amino acid sequence derived from the amino acid sequence as shown in SEQ ID NO: 2, 4, 6, or 8 by deletion, substitution, or addition of one or several amino acids and having activity of increasing seed size; or
(c) a protein comprising an amino acid sequence having 90% or higher identity with the amino acid sequence as shown in SEQ ID NO: 2, 4, 6, or 8 and having activity of increasing seed size.

10. A method for producing a transgenic plant comprising introducing any of DNAs (d) to (g) below into a plant cell and cultivating a plant:
(d) DNA comprising the nucleotide sequence as shown in SEQ ID NO: 1, 3, 5, or 7;
(e) DNA comprising a nucleotide sequence derived from the nucleotide sequence as shown in SEQ ID NO: 1, 3, 5, or 7 by deletion, substitution, or addition of one or several nucleotides and encoding a protein having activity of increasing seed size;
(f) DNA comprising a nucleotide sequence having 90% or higher identity with the nucleotide sequence as shown in SEQ ID NO: 1, 3, 5, or 7 and encoding a protein having activity of increasing seed size; or
(g) DNA hybridizing, under stringent conditions, to DNA comprising a nucleotide sequence complementary to DNA consisting of the nucleotide sequence as shown in SEQ ID NO: 1, 3, 5, or 7 and encoding a protein having activity of increasing seed size.

11. A method for producing a transgenic plant comprising introducing DNA encoding any of proteins (h) to (j) below into a plant cell and cultivating a plant:
(h) a protein comprising the amino acid sequence as shown in SEQ ID NO: 9, 10, or 11;
(i) a protein comprising an amino acid sequence derived from the amino acid sequence as shown in SEQ ID NO: 9, 10, or 11 by deletion, substitution, or addition of one or several amino acids and having activity of increasing seed size; or
(j) a protein comprising an amino acid sequence having 90% or higher identity with the amino acid sequence as shown in SEQ ID NO: 9, 10, or 11 and having activity of increasing seed size.

12. The method according to any one of claims 9 to 11, wherein DNA is introduced with the use of the recombinant vector according to any of claims 6 to 8.
